(19) United States
(12) Patent Application Publication
(10) Pub. No.: US 2019/0137505 A1
(43) Pub. Date: May 9, 2019

(54) BIOMARKERS OF METHYLGLYOXAL AND RELATED METHODS THEREOF

(71) Applicant: THE ARIZONA BOARD OF REGENTS ON BEHALF OF THE UNIVERSITY OF ARIZONA, Tucson, AR (US)

(72) Inventor: Serrine S. LAU, Tucson, AZ (US)

(21) Appl. No.: 15/751,759

(22) PCT Filed: Aug. 10, 2016
(86) PCT No.: PCT/US2016/046383

§ 371 Date: Feb. 9, 2018

Related U.S. Application Data
(60) Provisional application No. 62/203,230, filed on Aug. 10, 2015.

(51) Int. Cl. G01N 33/64; G01N 33/68; G01N 33/86

(52) U.S. Cl. CPC G01N 33/64; G01N 33/6893; G01N 33/86; G01N 2800/224; G01N 2333/765; G01N 2800/042; G01N 2800/52

(57) ABSTRACT

The present invention relates to methods and biomarkers for detection, characterization and treatment of conditions associated with methylglyoxal (MG) in biological samples. In particular, the present invention provides compositions and methods for determining diabetic complication onset in a patient through detecting a o-phenylenediamine derivatized MG (2MQ) product as indicative of the presence of MG, impaired fibrinolysis in a patient through detecting a MG modified plasminogen (Pg) product as indicative of impaired fibrinolysis, and the efficacy of metformin (MF) treatment in a patient through detecting IMZ as indicative of a MF/MG product.

### CROSS REFERENCE TO RELATED APPLICATION

This application claims priority to and the benefit of U.S. Provisional Application No. 62/203,230, filed Aug. 10, 2015, which is hereby incorporated by reference in its entirety.

### FIELD OF THE INVENTION

The present invention relates to methods and biomarkers for detection, characterization and treatment of conditions associated with methylglyoxal (MG) in biological samples. In particular, the present invention provides compositions and methods for determining diabetic complication onset in a patient through detecting a o-phenylenediamine derivatized MG (2MQ) product as indicative of the presence of MG, impaired fibrinolysis in a patient through detecting a MG modified plasminogen (Pg) product as indicative of impaired fibrinolysis, and the efficacy of metformin (MF) treatment in a patient through detecting IMZ as indicative of a MF/MG product.

### INTRODUCTION

Diabetes is a group of diseases marked by high levels of blood glucose resulting from defects in insulin production, insulin action, or both. Left untreated, it can cause many serious short term complications including symptoms of hypoglycemia, ketoacidosis, or nonketotic hyperosmolar coma. In the long term, diabetes is known to contribute to an increased risk of arteriosclerosis, chronic renal failure, retinal damage (including blindness), nerve damage and microvascular damage.

The spreading epidemic of diabetes in the developing world is predicted to have a profound impact on the healthcare system in the United States. A recent study by the Center for Disease Control and Prevention indicates the incidence of new diabetes cases in the U.S. nearly doubled in the last 10 years. As of 2007, at least 57 million people in the United States have pre-diabetes. Coupled with the nearly 24 million who already have diabetes, this places more than 25% of the U.S. population at risk for further complications from this disease. According to the American Diabetes Association, the estimated cost of diabetes in the United States in 2007 amounted to $174 billion with direct medical costs approaching $116 billion.

For the forgoing reasons, there is an unmet need for rapid and accurate diagnostic assays for the diagnosis and monitoring of patients at risk of developing diabetes and related complications.

### SUMMARY OF THE INVENTION

Experiments conducted during development of embodiments for the present invention determined that site-specific arginine dicarbonyl modifications of serum albumin are reduced in metformin-controlled T2DM patients. Advanced glycation end products (AGE) are a possible link between hyperglycemia and the development of diabetic complications. Careful control of AGE could be vital to improve diabetic complications. Biomarkers to evaluate control of AGE are needed to guide development of emerging therapies. Using an unbiased proteomics approach, dicarbonyl (methylglyoxal, glucosone, and 3-deoxyglucosone) on arginine (R) and oxidative markers on methionine, tryptophan, and cysteine (MWC) residues on serum albumin were studied in 116 subjects with varying degrees of type-2 diabetes mellitus (T2D), with or without metformin treatment. Trypsin-digested delipidated serum samples were analyzed by multiple reaction monitoring (MRM) on a QTRAP mass spectrometer, based on transitions that were validated in vitro. Strong signals for dicarbonyl modification were observed at R186, R257, and R428. Dicarbonyl and oxidative markers were significantly higher in T2D subjects (n=70) compared to non-diabetic subjects (n=46). In addition, dicarbonyl markers were significantly decreased in T2D subjects taking metformin (n=37) compared to T2D subjects not taking metformin (n=33 [Fisher's LSD]). Conversely, HbA1c was not significantly associated with metformin treatment among T2D patients. The study reveals that both dicarbonyl and oxidative biomarkers may be useful tools for monitoring the progression of T2D and metformin treatment efficacy. These markers identify biochemical differences associated with metformin treatment that are not reflected in HbA1c levels.

Methylglyoxal (MG)—the aldehyde form of pyruvic acid, also called pyruvaldehyde or 2-oxopropanal, with the formula: (CH₃—CO—CH═O or C₃H₄O₂)—is a unique but ubiquitous molecule present in most biological systems including all mammalian cells (see, Inoue, Adv Microb Physio 1995). It is a highly reactive and dose-dependent cytotoxic metabolite that is primarily produced during glycolysis, a key metabolic step for respiring organisms.

Methylglyoxal is a highly reactive dicarbonyl compound involved in the formation of advanced glycation endproducts. Levels of methylglyoxal have been found elevated in patients with type 2 diabetes (T2D) and advanced glycation endproducts (AGEs) have been implicated in the progression of diabetic complications.

Experiments conducted during the course of developing embodiments for the present invention identified IMZ as indicative of a MF/MG product. Indeed, experiments conducted during development of embodiments for the present invention determined that a cyclized imidazolinone derivative (IMZ) is the predominant methylglyoxal scavenging product from the anti-hyperglycemic drug metformin. Metformin, a first line therapy for the treatment of hyperglycemia in T2D patients, has been suggested to be a scavenger of methylglyoxal. Experiments were conducted that examined and characterized unequivocally the resulting scavenged product from the metformin-methylglyoxal reaction. The primary product, a white precipitate with a molecular weight of 183.22 Da, was characterized with ¹H, ¹³C, 2D-HSQC and HMBC NMR as well as tandem mass spectrometry. This product was subsequently re-crystallized in a 1:1 dimethylformamide:acetonitrile solution and crystals underwent X-ray diffraction analysis. The product structure was determined to be (E)-1,1-dimethyl-2-(5-methyl-4-oxo-4,5-dihydro-1H-imidazol-2-yl)guanidine for this metformin and methylglyoxal-derived imidazolinone compound. A method was developed utilizing LC-MS/MS using multiple reaction monitoring mode to detect and quantify the presence of this adduct in patients treated with metformin. A subset of human diabetic urine samples analyzed with this assay indicates the presence of this imidazolinone product in every patient treated with metformin, suggesting a possible secondary mechanism of drug efficacy. In addition to lowering hepatic gluconeogenesis, metformin was shown to perform a role in scavenging the highly reactive methylglyoxal in vivo, thereby preventing diabetic complications resulting from AGE formation.

Accordingly, in certain embodiments, the present invention provides methods for determining the efficacy of metformin (MF) treatment in a patient, comprising a) obtaining a urine sample from the patient; b) detecting whether a MF/methylglyoxal (MG) product is present in the urine sample by contacting the urine sample with an agent that binds a MF/MG product and detecting binding between the MF/MG product and agent that binds a MF/MG product; and c) determining the MF treatment as efficacious when the presence of the MF/MG product in the urine sample is detected.

In some embodiments, the patient is a human patient. In some embodiments, the patient is a human patient diagnosed with type 2 diabetes (T2D).

In some embodiments, the MF/MG product is a MF/MG imidazolinone product (IMZ). In some embodiments, the agent that binds a MF/MG product is an anti-MF/MG product antibody. In some embodiments, the agent that binds a MF/MG product is an anti-MF/MG product small molecule.

Experiments conducted during the course of developing embodiments for the present invention identified a o-phenylenediamine derivatized MG (2MQ) product as indicative of the presence of MG.

Accordingly, in certain embodiments, the present invention provides methods for diagnosing diabetic complication development in a patient, comprising a) obtaining a urine sample from the patient; b) detecting whether a o-phenylenediamine derivatized methylglyoxal (MG) (2MQ) product is present in the urine sample by contacting the urine sample with an agent that binds a 2MQ product and detecting binding between the 2MQ product and agent that binds a 2MQ product; and c) diagnosing the patient has having diabetic complication development when the presence of the 2MQ product in the urine sample is detected.

In some embodiments, the patient is a human patient. In some embodiments, the patient is a human patient diagnosed with type 2 diabetes (T2D).

In some embodiments, the agent that binds a 2MQ product is an anti-2MQ product antibody. In some embodiments, the agent that binds a 2MQ product is an anti-2MQ product small molecule.

In some embodiments, the diabetic complication is one or more of diabetic retinopathy, diabetic neuropathy, diabetic cardiovascular disease, and diabetic nephropathy.

Experiments identified a MG modified plasminogen (Pg) product as indicative of impaired fibrinolysis. Indeed, experiments conducted during development of embodiments for the present invention determined that sensitive sites of glycation of the fibrinolytic system protein plasminogen and implications in the progression of type 2 diabetes cardiovascular complications. Reactive dicarbonyls, such as methylglyoxal (MG), are present in blood and react with arginines (R) of target proteins, leading to diabetic micro- and macrovascular complications. MG plasma concentrations reach 4.5 μM, which can triple as diabetic complications progress. Protein-MG adducts may drive retinopathy, neuropathy, and many other common diabetic complications. The irreversible modification of R residues by dicarbonyls causes a net loss of positive charge, most commonly via hydroimidazolone formation. Previous studies identified the fibrinolytic system protein plasminogen (Pg) as a sensitive target of MG adduction, with functional significance. Thus, molecular modeling indicated that modification of R561 at the cleavage site of Pg would impact enzymatic activation due to drastically altered energy of interaction. The current work has identified, via 2D gel and subsequent in-gel digestion and liquid chromatography coupled to high resolution tandem mass spectrometry (LC-MS/MS), the most sensitive sites of R modification (R504, R530) by MG on the protein, both of which may have functional effects. Although R561 was found as an early target for modification, it was not amongst the first sites hit. The functional significance of Pg adduction by MG was further tested in vitro. Pg activation was significantly delayed via modification with 100 μM MG, as determined by cleavage of a chromogenic substrate by plasmin (Pn), the product of Pg activation. A group of sixteen human plasma samples were selected and analyzed via LC-MS/MS on a LTQ Orbitrap Velos mass spectrometer to detect modifications. Nine patients showed R modification at a number of R sites. The findings indicate that MG-modification of Pg may disrupt the fibrinolytic cascade sufficient to represent an underlying mechanism of vascular complications in diabetics.

Accordingly, in certain embodiments, the present invention provides methods for diagnosing impaired fibrinolysis in a patient, comprising a) obtaining a biological sample from the patient, b) detecting whether an methylglyoxal (MG) modified plasminogen (Pg) product is present in the biological sample by contacting the biological sample with an agent that binds a MG/Pg product and detecting binding between the MG/Pg product and agent that binds a MG/Pg product; and c) diagnosing impaired fibrinolysis in the patient when the presence of the MG/Pg product in the biological sample is detected.

In some embodiments, the patient is a human patient. In some embodiments, the patient is a human patient diagnosed with type 2 diabetes (T2D).

In some embodiments, the agent that binds a MG/Pg product is an anti-MG/Pg product antibody. In some embodiments, the agent that binds a MG/Pg product is an anti-MG/Pg product small molecule.

In some embodiments, detected fibrinolysis is indicative of thrombosis.

Additionally, provided herein is a technology relating to the interaction between metformin and methylglyoxal. In particular, the present invention provides methods, compositions, and related uses for the interaction between metformin and methylglyoxal. In addition, the present invention provides methods for detecting the efficacy of metformin therapy.

For example, in some embodiments, the present invention provides a method of assaying a sample from a subject for the presence of advanced glycation end products (AGE), comprising: a) contacting the sample with an assay for determining dicarbonyl modification and/or oxidation of serum albumin; and b) determining the presence of dicarbonyl modification of one or more arginine residues on the serum albumin and/or oxidation of one or more methionine residues on the serum albumin. In some embodiments, the arginine residues are one or more of R186, R257, and R428. In some embodiments, the assay is a mass spectrometry assay. In some embodiments, the subject has type 2 diabetes. In some embodiments, the subject is currently taking metformin. In some embodiments, the dicarbonyl modification is increased in subjects with type 2 diabetes relative to subjects not having type 2 diabetes. In some embodiments, dicarbonyl modification is decreased in subjects taking metformin relative to the level in subjects with type 2 diabetes not taking metformin. In some embodiments, decrease in dicarbonyl modification is indicative of metformin being an effective treatment for said type 2 diabetes. In some embodiments, the dicarbonyl is selected from, for example, methylglyoxal, 3-deoxyglucosone, and glucosone.

Further embodiments provide a method of monitoring and/or predicting diabetic complications, comprising: a) assaying methylglyoxal adduction of plasminogen in a biological sample from the subject; and b) determining an increased risk and/or presence of diabetic complications when the adduction is identified. In some embodiments, adduction is arginine adduction of one or more of R504, R530, R561 of plasminogen. In some embodiments, the adduction prevents cleavage of plasminogen into plasmin. In some embodiments, the adduction decreases at least one biological activity of the plasminogen. In some embodiments, the subject has type 2 diabetes. In some embodiments, the subject is currently taking metformin. In some embodiments, the assaying comprises mass spectrometry.

Additional embodiments provide a method of monitoring diabetes treatment in a type 2 diabetic subject treated with metformin, comprising: a) identifying the level of o-phenylenediamine derivatized methylglyoxal or MG-metformin imidazolinone in a sample from the subject; and b) determining the efficacy of the treatment based on said level. In some embodiments, the sample is a urine sample. In some embodiments, the assay is a LC/MS multiple reaction monitoring assay. In some embodiments, an increased level of said o-phenylenediamine derivatized methylglyoxal or MG-metformin imidazolinone is indicative of treatment with metformin being effective.

Further embodiments provide kits and systems for performing the above-described methods (e.g. comprising one or more of reagents, mass spectrometry systems, chromatography systems, standards, computer systems, etc.).

Experiments conducted during development of embodiments for the present invention determined that a cyclized imidazolinone derivative is the predominant methylglyoxal scavenging product from the anti-hyperglycemic drug metformin. Methylglyoxal is a highly reactive dicarbonyl compound involved in the formation of advanced glycation endproducts. Levels of methylglyoxal have been found elevated in patients with type 2 diabetes (T2D) and advanced glycation endproducts (AGEs) have been implicated in the progression of diabetic complications. Metformin, a first line therapy for the treatment of hyperglycemia in T2D patients, has been suggested to be a scavenger of methylglyoxal. The present work examined and characterized unequivocally the resulting scavenged product from the metformin-methylglyoxal reaction. The primary product, a white precipitate with a molecular weight of 183.22 Da, was characterized with ¹H, ¹³C, 2D-HSQC and HMBC NMR as well as tandem mass spectrometry. This product was subsequently re-crystallized in a 1:1 dimethylformamide:acetonitrile solution and crystals underwent X-ray diffraction analysis. The product structure was determined to be (E)-1,1-dimethyl-2-(5-methyl-4-oxo-4,5-dihydro-1H-imidazol-2-yl)guanidine

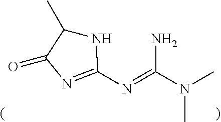

for this metformin and methylglyoxal-derived imidazolinone compound. A method was developed utilizing LC-MS/MS using multiple reaction monitoring mode to detect and quantify the presence of this adduct in patients treated with metformin. A subset of human diabetic urine samples analyzed with this assay indicates the presence of this imidazolinone product in every patient treated with metformin, suggesting a possible secondary mechanism of drug efficacy. In addition to lowering hepatic gluconeogenesis, metformin may play a role in scavenging the highly reactive methylglyoxal in vivo, thereby preventing diabetic complications resulting from AGE formation.

Accordingly, provided herein is a technology relating to a metformin and methylglyoxal-derived imidazolinone compound, and related methods of use. In particular, the present invention provides compositions comprising a metformin and methylglyoxal-derived imidazolinone compound for purposes of treating type 2 diabetes and related complications, and assessing the effectiveness of treating type 2 diabetes with metformin.

For example, in some embodiments, the present invention provides a method of treating, ameliorating, or preventing type 2 diabetes, comprising: administering a metformin and methylglyoxal-derived imidazolinone compound or a derivative or mimetic thereof to a subject diagnosed with diabetes. In some embodiments, the metformin and methylglyoxal-derived imidazolinone compound inhibits one or more biological activities of methylglyoxal. In some embodiments, the biological activity is methylglyoxal related adduction of plasminogen or albumin. In some embodiments, the metformin and methylglyoxal-derived imidazolinone compound is

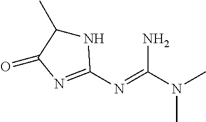

or compounds having a structure similar to such a compound.

In certain embodiments, the present invention provides a method of treating, ameliorating, or preventing complications associated with type 2 diabetes, comprising: administering MG-metformin imidazolinone or a derivative or mimetic thereof to a subject diagnosed with diabetes. In some embodiments, the metformin and methylglyoxal-derived imidazolinone compound or a derivative or mimetic thereof inhibits one or more biological activities of methylglyoxal. In some embodiments, the biological activity is methylglyoxal related adduction of plasminogen or albumin. In some embodiments, the imidazolinone compound is

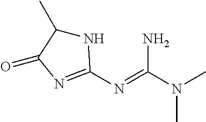

or compounds having a structure similar to such a compound. In some embodiments, the complications associated with type 2 diabetes include, but are not limited to, neuropathy, nephropathy, retinopathy, atherosclerosis, etc.

In certain embodiments, the present invention provides methods of monitoring the treatment of type 2 diabetes in a human subject with metformin, the method comprising obtaining a urine sample from a human subject having type 2 diabetes being treated with metformin; determining the levels of

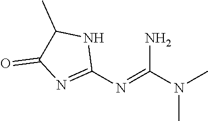

in the urine sample; comparing the determined levels of

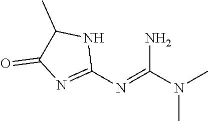

with reference levels of

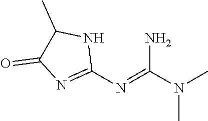

in urine samples from type 2 diabetic patients responding favorably to metformin treatment; identifying the treatment as a favorable treatment response if the determined levels of

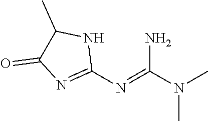

are consistent or higher than the reference levels, or identifying the treatment as an unfavorable treatment response if the determined levels of

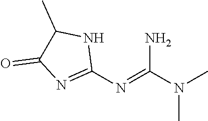

are lower than the reference levels. In some embodiments, the methods further comprise communicating the effectiveness of the treatment to the subject or a health care provider.

In further embodiments, the present disclosure provides a method of treating cancer, comprising: administering a metformin and methylglyoxal-derived imidazolinone compound or a derivative or mimetic thereof to a subject diagnosed with cancer. In some embodiments, the imidazolinone compound is

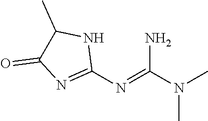

or compounds having a structure similar to such a compound.

In certain embodiments, the present invention contemplates that exposure of animals (e.g., humans) suffering from type 2 diabetes (e.g., and/or related complications) to therapeutically effective amounts of a metformin and methylglyoxal-derived imidazolinone compound

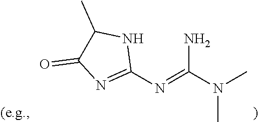

or a derivative or mimetic thereof will result in inhibition of type 2 diabetes and/or complications related to type 2 diabetes. The present invention contemplates that therapeutically effective amounts of a metformin and methylglyoxal-derived imidazolinone compound

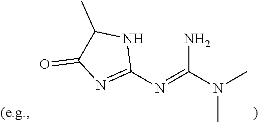

or a derivative or mimetic thereof satisfy an unmet need for the treatment of type 2 diabetes and/or complications related to type 2 diabetes, either when administered as monotherapy, or when administered in a temporal relationship with additional agent(s) (combination therapies).

In certain embodiments of the invention, combination treatment of animals with a therapeutically effective amount of a metformin and methylglyoxal-derived imidazolinone compound

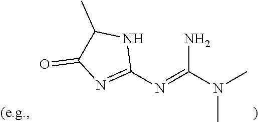

or a derivative or mimetic thereof and a course of an additional agent produces a treatment response and clinical benefit in such animals compared to those treated with the additional agent alone. Since the doses for all approved drugs for treating type 2 diabetes are known, the present invention contemplates the various combinations of them with the present compounds.

The invention also provides pharmaceutical compositions comprising a metformin and methylglyoxal-derived imidazolinone compound

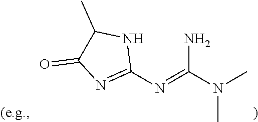

or a derivative or mimetic thereof in a pharmaceutically acceptable carrier.

The invention also provides kits comprising a metformin and methylglyoxal-derived imidazolinone compound

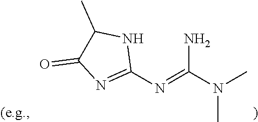

or a derivative or mimetic thereof and instructions for administering the compound to an animal. The kits may optionally contain other therapeutic agents, e.g., additional agents for treating type 2 diabetes and/or complications related to type 2 diabetes.

Examples of complications related to type 2 diabetes include, but are not limited to, diabetic neuropathy, diabetic nephropathy, diabetic atherosclerosis, and diabetic retinopathy.

Examples of additional agents known for treating type 2 diabetes and/or complications related to type 2 diabetes include, but are not limited to, biguanides (e.g., metformin), sulfonylureas (e.g., glyburide, glipizide, and glimepiride), meglitinide derivatives (e.g., repaglinide, nateglinide), alpha-gluocsidase inhibitors (e.g., acarbose, miglitol), thiazolidinediones (e.g., pioglitazone, rosiglitazone), glucagonlike peptide-1 agonists (e.g., exenatide, liraglutide, albiglutide, dulaglutide), dipeptidyl peptidase IV inhibitors (e.g., sitagliptin, saxagliptin, linagliptin, alogliptin), amylinomimetics (e.g., pramlintide), sodium-glucose transporter-2 inhibitors (e.g., canagliflozin, dapagliflozin, empagliflozin), bile acid sequestrants (e.g., colesevelam), rapid acting insulins (e.g., insulin aspart, insulin glulisine, insulin lispro, insulin inhaled), short acting insulins (e.g., regular insulin), intermediate acting insulins (e.g., insulin NPH), long acting insulins (e.g., insulin detemir, insulin glargine, insulin degludec), antiparkinson agents/dopamine agonists (e.g., bromocriptine), angiotensin receptor blockers (e.g., captopril, enalapril, lisinopril, losartan, valsartan, irbesartan), beta-adrenergic blocking agents (e.g., metoprolol, atenolol, labetalol), calcium channel blockers (e.g., diltiazem, verapamil, nifedipine, amlodipine), diuretics (e.g., furosemide, hydrochlorothiazide, bumetanide), direct renin inhibitors (e.g., aliskiren), sodium-glucose cotransporter 2 inhibitors (e.g., canagliflozin), nonsteroidal anti-inflammatory drugs (e.g., ibuprofen, naproxen), analgesics (e.g., capsaicin cream), anticonvulsants (e.g., gabapentin, carbamazepine, pregabalin, phenytoin), antidepressants (e.g., amitriptyline, imipramine, nortriptyline, duloxetine, citalopram, paroxetine, desipramine), antiarrhythmic agents (e.g., lidocaine anesthetic), prokinetic agents (e.g., erythromycin, cisapride, metoclopramide), synthetic adrenocortical steroids (e.g., fludrocortisone acetate), cholinergic agents (e.g., bethanechol hydrochloride), laxatives (e.g., polyethylene glycol solution), corticosteroids (e.g., triamcinolone acetonide), ophthalmics (e.g., ranibizumab, aflibercept intravitreal), HMG-CoA reductase inhibitors (e.g., pravastatin, simvastatin, lovastatin, fluvastatin, atorvastatin, rosuvastatin, pitavastatin), and fabric acid derivatives.

Additional embodiments provide pharmaceutical compositions comprising the above-described agents, alone or in combination with metformin.

Further embodiments are described herein.

### BRIEF DESCRIPTION OF THE DRAWINGS

FIG. 1: Formation of IMZ through MF and MG interaction.

FIG. 2: MG irreversibly adducts arginine residues in proteins which subsequently alter the charge status.

FIG. 3: Formation of derivatized methylglyoxal (2MQ).

FIG. 4: Activation of MG-modified Pg. Activation of MG-modified Pg was tested utilizing a chromogenic substrate which when cleaved is detectable at 405 nm. (A) Tissue plasminogen activator (tPA) at a concentration of 12 nM was utilized to activate 24-hour unmodified or 50-500 μM MG-modified Pg. Significant activation delays began to be observed at 30 minutes for 100 μM modification and 28 minutes for 500 μM modification (*=p<0.05). (B) Urokinase plasminogen activator (uPA) at a concentration of 50 nM was utiltized to activate 24-hour hour unmodified or 50-500 μM MG-modified Pg. The only statistically significant delay occurred with 500 μM modified Pg, beginning at the 37 minute mark (*=p<0.05). (C) Streptokinase (STK) at a concentration of 5 nM was utilized to activate 24-hour hour unmodified or 50-500 μM MG-modified Pg. Significant activation delays began to be observed at 16 minutes for 100 μM modification and 12 minutes for 500 μM modification (*=p<0.05).

FIG. 5: Two-dimensional gel analysis of MG-modified Pg. Isoelectric focusing strips (4-7 pH) were utilized to capture pI changes due to MG-modification of Pg. (A) 10 μg unmodified glu-Pg as a pI equivalent to 6.5-6.8. The molecular weight appears just above the 100 kDa molecular weight marker, which has been shown to be correct experimentally despite a known molecular weight of 91 kDa. Spots L and M were cut as shown in the figure and analyzed via mass spectrometry. (B) 10 μg of 24-hr 500 μM MG-modified Pg focused in an extremely wide band, consisting of multiple spots, from a pI of 5.8-6.7. The band appeared at the correct molecular weight. The long band was excised in multiple portions as indicated in the figure. Bands A-K were submitted for mass spectrometry analysis. A portion of the wide protein spot, between spots D and E, was discarded from the study due to contamination in a post-processing step.

FIG. 6: MG-H1 modified R504 peptide from patient RS104. A model MS/MS spectrum of all MG-H1 modified peptides is shown. In particular, the peptide containing modified R504 is shown. A large number of b- and y-fragment ions cover the site of modification. The modification at R504 results in a mass shift at the y-6 and higher y fragment ions. Furthermore, b-11 and b-12 fragment ions are also present, which include the modified residue. Additional less common ion peaks are detected as well, including +2H versions of common ions, that confirm the modification. An analogous unmodified peptide was undetected in the patient sample, despite 84% peptide coverage of the protein. The comparable 16-amino acid version of this peptide (with an unmodified arginine) does not exist in the sample, as cleavage occurs after R504 at the 11 position. A large number of these 11-amino acid peptides were detected.

FIG. 7: Structures for A) methyglyoxal, B) Metformin, C) metformin-methyglyoxal imidazolinone, and D) triazepinone.

FIG. 8: Two possible structures for the metformin-methylglyoxal reaction product. Carbon numbering from structure 1 was transferred to analogous positions in structure 2 for ease of comparison. Continuous arrows indicate selected observed HMBC correlations; arrows marked with an “X” correspond to HMBC correlations that are not observed.

FIG. 9: HMBC data rules out the possibility of a seven-membered triazepinone. 2D-HMBC of the metformin-MG product indicates a non-triazepinone based compound. Lower (F₁ downfield) region of 2D-HMBC spectrum of the metformin/methylglyoxal reaction product in DMSO-d₆. In the inset (upper left) the contour threshold is reduced to show the noise floor. The signal-to-noise ratio of the H11/12 peak in an F2 trace at C7 is greater than 1000:1, with no detectable peak at H2.

FIG. 10: Placement of double bonds by the software Mercury ultimately led to the conclusive determination of the structure as (E)-1,1-dimethyl-2-(5-methyl-4-oxo-4,5-dihydro-1H-imidazol-2-yl)guanidine.

FIG. 11: LC-MS/MS MRM identification of the imidazolinone product. Three parent compounds—metformin-MG imidazolinone (A), metformin (B), and metformin-D₆ (C)—are monitored for in a metformin-treated T2D urine sample. Multiple fragment peaks, as detailed in Table 4, are monitored for, though only one representative peak is shown for each compound in the figure. The retention time is indicated at the top of each peak.

FIG. 12: Oxidation of methionine (M) and corresponding masses. The sulfur group is easily oxidized to form the sulfoxide (+16) and prolonged oxidation will produce sulfone (+32).

FIG. 13: Major oxidation products of tryptophan (W) and the corresponding mass changes. The C2-C3 double bond of the indole ring on the side chain of tryptophan is initially oxidized to oxindolyalanine and its tautomer 2-hydroxytryptophan. Further oxidation cleaves the C2-C3 bond to yield N-formlykynurenine. Hydrolysis of the n-formyl group generates formic acid and the advanced oxidation product kynurenine.

FIG. 14: Modification of cysteine (C). During sample preparation of protein to peptides, cysteine is commonly treated with iodoacetamide to cap the reactive thiol (C+57, top scheme). Cysteine may be oxidized (left pathway) to the intermediate sulfenic acid (C+16) or to the sulfone (C+32) or to the sulfonic acid (C+48). Oxidized cysteines, such as sulfone and sulfonic acid are not reduced by DTT, so they are not capped by iodoacetamide during sample prep.

FIG. 15: Hydroimidazolone adducts from dicarbonyl modification of arginine and the corresponding mass changes. Arrows represent arginine adduction. Dicarbonyls glucosone, 3DG, and MG will target arginine residues in proteins to yield corresponding mass increases of R+160, R+144, and R+54, respectively.

FIG. 16: Hierarchical clustering of the AUC values for the three dicarbonyls at three R sites. Dendrogram produced from clustering the log transformed transition averages of MG, glucosones (GN), and 3DG at three specific sites of HSA modification (R186, R257, R428). Hierarchical clustering was performed with complete linkage and with 1−r² as the distance metric, where r is the Pearson correlation coefficient. Hierarchical clustering of the AUC values for the three dicarbonyls at three R sites. Log transformed MRM AUC values from each subject in technical duplicates were clustered. MG sites R186 and R257 clustered together, and the values were averaged between the two sites for each subject. 3DG and glucosone sites R186 and R428 clustered together, and these four values were averaged to give a single “normalized glucosones” value for each subject.

FIG. 17: Group differences in oxidative modifications, normalized MG and glucosone modifications, and ranked HbA1c. Notice p-values for pairwise differences between subject groups (nondiabetic, T2D not taking Metformin, and T2D with Metformin). The mean HSA modifications for T2D with Metformin are lower than those for T2D not taking Metformin; this “metformin effect” is significant in MG and glucosone modifications. No such difference between these groups exists for HbA1c.

FIG. 18: Stoke's shift of HSA-prodan complex yields fluorescent maxima at 465 nm. HSA was incubated with a 10-fold excess of prodan and allowed to equilibrate for 30 minutes. Unbound prodan was removed by acetone precipitation and the emission spectra of free prodan, HSA-prodan and HSA were obtained. Excitation λ is 380 nm and upon binding HSA, prodan λmax blue-shifts from 520 nm to 465 nm. HSA without prodan (bottom spectra) shows negligible spectral emission. The endpoint values to monitor prodan displacement from the HSA-prodan complex are 380 nm excitation, 465 nm emission.

FIG. 19: Prodan is displaced from drug site I by MG modification of HSA. Prodan bound to HSA (prodan-HSA complex, 75 μM) was treated with a dilution of MG and the fluorescence (excitation 380 nm, emission 465 nm, filter cut-off 420 nm) was measured after 30 minutes. Means±SD for three separate experiments are given. Significant (*p<0.05; **p<0.01; ***p<0.001) as compared to control (HSA without MG treatment).

FIG. 20: Human plasma samples indicate the presence of modified R257. (A) Multiple-reaction monitoring transitions (660.6/237.1; 660.6/109.6; 660.6/801.5; 660.6/635.6) indicate the presence of a modified R257-containing peptide of albumin in the plasma of human patients, shown in a representative diabetic patient sample. (B) Four separate patient plasma samples, representative of all 12 patients analyzed, showed the presence of 660.6/237.1 transition.

FIG. 21: Arginine and dicarbonyl-derived adducts. Arginine, argpyrimidine, MG-H1, and 3DG-H1 chemical structures and chemical names.

FIG. 22: Molecular modeling of Pg illustrating R561-D189 salt bridge. The normal molecular model (A) shows a salt bridge between cleavage-site arginine-561 of Pg and aspartic acid-189 of tPA at 2.746 or 3.033 Å. A salt bridge has a distance cutoff of 4 Å. In the model for argpyrimidine (B), MG-H1 (C) and 3DG-H1 (D), the salt bridge is unable to form. Images were created with the use of Accelrys Discovery Studio 3.1 Visualizer.

FIG. 23: Molecular modeling of Pg illustrating V562-H57 contact distance. The normal molecular model (A) shows that the β-carbons of valine-562 of Pg and catalytic histidine-57 of tPA are within the contact distance of 8 Å. In the R561 modified model for argpyrimidine (B), MG-H1 (C) and 3DG-H1 (D), the contact distance is beyond the 8 Å threshold for contact distance. Images were created with the use of Accelrys Discovery Studio 3.1 Visualizer.

FIG. 24: Interpolated charge surface area of Pg basic cluster and tPA acidic cluster. The interpolated surface of the interaction between K556, K557, and H569 of Pg with D95, D96, and D97 of tPA. The normal model (A) displays the basic (blue) and acidic (red) clusters as expected, from two different views. Salt bridges between K556-D96 and K557-D97 are possible in the normal model. The modified R561 model (B) with argpyrimidine indicates a change in this interaction. The cluster is now almost entirely acidic, and no salt bridges are possible. MG-H1 (C) and 3DG-H1 (D) models exhibit a similar cluster change to the argpyrimidine model. Images were created with the use of Accelrys Discovery Studio 3.1 Visualizer.

FIG. 25: Tertiary structure modulation due to MG-H1 adduction at R504. The overall change in tertiary structure due to R504 modification was minimal, although slight changes between unmodified (purple) and modified (green) are observed. Most notably, there is a difference in folding observed just to the right (C-terminal side) of the modification. A similar change was observed with argpyrimidine modification at the R504 (B). Images were created with the use of Accelrys Discovery Studio 4.0 Visualizer.

FIG. 26: Change in interpolated charge on kringle 5 domain of Pg. The normal interpolated charge (A) on kringle 5 domain, with the unmodified R504 residue visible as a stick structure. MG-H1 modification at this residue (B) not only causes a local change in charge state from basic to neutral, but an overall neutralization of the surrounding area on the molecule. Images were created with the use of Accelrys Discovery Studio 4.0 Visualizer.

FIG. 27: Change in binding pocket of Pg kringle 5 domain. The unmodified model binding pocket (A) has the correct orientation of D516 and D518. Unmodified R504 is indicated in yellow. The modified model has an overall expansion of the binding pocket, most notably due to a shift in orientation of D516. MG-H1 modified R504 is indicated in purple. D516/D518 CY carboxylate distances are noted in blue. Images were created with the use of Accelrys Discovery Studio 4.0 Visualizer.

FIG. 28: Streptokinase cleavage of Pg. Unmodified and 24-hr 100 μM MG-modified glu-Pg were reacted with STK for 0 to 60 min. Unmodified (left) Pg exhibits the presence of Pg at 100 kDa and multiple breakdown products, including bands consistent with plasmin heavy (HC) and light chain (LC), and angiostatin. Modified (right) Pg exhibits only the presence of a Pg band at 100 kDa but no breakdown products. Blot was imaged using a ChemiDoc XRS+ (Life Sciences Research).

FIG. 29: Activation of MG-modified Pg. Activation of PG and MG-modified Pg was tested utilizing a chromogenic substrate which when cleaved is detectable at 405 nm. (A) Streptokinase (STK) at a concentration of 5 nM was utilized to activate 24-hour unmodified or 50-500 μM MG-modified Pg. Significant activation delays began to be observed (shown within the inset) at 16 minutes for 100 μM modification and 12 minutes for 500 μM modification (*=p<0.05). (B) Tissue plasminogen activator (tPA) at a concentration of 12 nM was utilized to activate 24-hour unmodified or 50-500 μM MG-modified Pg. Significant activation delays (shown within the inset) began to be observed at 30 minutes for 100 μM modification and 28 minutes for 500 μM modification (*=p<0.05). (C) Urokinase plasminogen activator (uPA) at a concentration of 50 nM was utiltized to activate 24-hour hour unmodified or 50-500 μM MG-modified Pg. The only statistically significant delay (shown within the inset) occurred with 500 μM modified Pg, beginning at the 37 minute mark (*=p<0.05).

FIG. 30: Effect of imidazoline on tRAPTOR and AAC.

FIG. 31: Generation of methylglyoxal from glucose.

FIG. 32: Schematic of adduction of arginine residues.

FIG. 33: Schematic of plasminogen-mediated fibrosis.

FIG. 34: Structure of metformin.

FIG. 35: Synthesis of MG-metformin IMZ.

FIG. 36: NMR analysis of MG-metformin IMZ.

FIG. 37: X-ray diffraction analysis of MG-metformin IMZ.

FIG. 38: Correlation of metformin levels with MG-metformin IMZ levels in human urine.

FIG. 39: Formation of derivatized methylglyoxal (2MQ).

FIG. 40: Multiple reaction monitoring (MRM) assay for the identification and quantitation of derivatized methylglyoxal (2MQ) levels in human urine.

FIG. 41: Chromatography trace of 2MQ and internal standard 5MQ.

### DEFINITIONS

The term “type 2 diabetes” refers to a metabolic disorder that is characterized by high blood glucose in the context of insulin resistance and relative insulin deficiency. Type 2 diabetes may be caused by a combination of lifestyle and genetic factors. Risk factors include but not limited to obesity, hypertension, high blood cholesterol, metabolic syndrome (high triglyceride, low HDL-C, high blood glucose, high blood pressure, large waist), endocrine disorders (e.g., Cushing's syndrome), chronic pancreatitis, use of certain drugs, aging, energy dense diets (e.g., high-fat and high glucose), and an inactive lifestyle. On the other hand, having relatives (especially first degree) with type 2 increases risks of developing type 2 diabetes substantially. Symptoms of type 2 diabetes often include polyuria (frequent urination), polydipsia (increased thirst), polyphagia (increased hunger), fatigue, and weight loss. Co-morbidities associated with type 2 diabetes due to high blood sugar and abnormal metabolic milieu (e.g., low grade inflammation, oxidative stress and abnormal lipids) include increased risk of heart attacks, strokes, limb amputation, visual loss, kidney failure, cancers, and cognitive impairment.

The term “cardiovascular disease” refers to a broad class of diseases that involve the heart or blood vessels (arteries and veins) and affect the cardiovascular system, such as conditions related to atherosclerosis (arterial disease). These include but not limited to stroke, coronary heart disease and peripheral vascular disease. Known risk factors for cardiovascular diseases include unhealthy eating, lack of exercise, obesity, improperly managed diabetes, abnormal blood lipids, high blood pressure, consumption of alcohol and/or tobacco, as well as genetic background.

In this disclosure the term “biological sample” or “sample” includes any section of tissue or bodily fluid taken from a test subject such as a biopsy and autopsy sample, and frozen section taken for histologic purposes, or processed forms of any of such samples. Biological samples include blood and blood fractions or products (e.g., serum, plasma, platelets, red blood cells, and the like), sputum or saliva, lymph and tongue tissue, cultured cells, e.g., primary cultures, explants, and transformed cells, stool, urine, stomach biopsy tissue etc. A biological sample is typically obtained from a eukaryotic organism, which may be a mammal, may be a primate and may be a human subject.

The term “immunoglobulin” or “antibody” (used interchangeably herein) refers to an antigen-binding protein having a basic four-polypeptide chain structure consisting of two heavy and two light chains, said chains being stabilized, for example, by interchain disulfide bonds, which has the ability to specifically bind antigen. Both heavy and light chains are folded into domains.

The term “antibody” also refers to antigen- and epitope-binding fragments of antibodies, e.g., Fab fragments, that can be used in immunological affinity assays. There are a number of well characterized antibody fragments. Thus, for example, pepsin digests an antibody C-terminal to the disulfide linkages in the hinge region to produce F(ab)′₂, a dimer of Fab which itself is a light chain joined to V_{H}-C_{H1} by a disulfide bond. The F(ab)′₂ can be reduced under mild conditions to break the disulfide linkage in the hinge region thereby converting the (Fab′)₂ dimer into an Fab′ monomer. The Fab′ monomer is essentially a Fab with part of the hinge region (see, Fundamental Immunology, Paul, ed., Raven Press, N.Y. (1993)). While various antibody fragments are defined in terms of the digestion of an intact antibody, one of skill will appreciate that fragments can be synthesized de novo either chemically or by utilizing recombinant DNA methodology. Thus, the term antibody also includes antibody fragments either produced by the modification of whole antibodies or synthesized using recombinant DNA methodologies.

### DETAILED DESCRIPTION OF THE INVENTION

Reactive dicarbonyls, such as methylglyoxal (MG), are elevated in type-2 diabetes mellitus (T2DM) patients, covalently modify proteins, and contribute to a number of diabetic complications. The T2DM first-line therapy metformin (MF) significantly reduces diabetic endpoints and mortality more effectively than other hypoglycemic agents.

Experiments conducted during the course of developing embodiments for the present invention resulted in detection of methyglyoxal in human urine. Such experiments successfully detected the quinoxaline derivative of methylglyoxal (2MQ) at a range of 0.01 μM to 5.5 μM without reaching saturation. Levels of 2MQ are detectable in urine samples from human diabetic subjects on metformin treatment within range of calibration.

Experiments were conducted that examined whether MF directly scavenges MG as an alternative mechanism of drug efficacy, in addition to its antigluconeogenesis mechanism. A MF/MG imidazolinone product (IMZ) was synthesized and characterized by 13C, 1H and HMBC NMR (Bruker DRX-600), X-ray diffraction analysis (Bruker APEX-II CCD) as well as ESI-LC-MS/MS (MH+, 184 m/z; Agilent 6490). Using an LC/MS multiple reaction monitoring (MRM) method experiments measured MF and the IMZ product in human urine with sensitivity in the nM range. The IMZ was detected in all MF-treated T2DM patients and not detected in patients that have not taken metformin, as expected. The data reveals that urine from every T2DM patient treated with MF contains the IMZ product as a result of a direct reaction with MG, and increased levels of MF directly correlate with elevations in IMZ (FIG. *1). The present work has identified an optimized method for detecting o-phenylenediamine derivatized MG (2MQ) using an LC/MS multiple reaction monitoring method. Utilizing this method, the 2MQ product can be detected at an average retention time of 4.3 minutes in a range between 0.01 μM to 5.504 without reaching saturation. MF may play a role in scavenging the highly reactive MG in vivo, in addition to lowering hepatic gluconeogenesis.

Reactive dicarbonyls such as methylglyoxal accumulate in diabetic patients due to elevated glucose as well as increased oxidative stress. These toxic dicarbonyls directly damage proteins through adduction at arginine residues on proteins (advanced glycation end products [AGEs]) and are implicated in the progression of a number of type-2 diabetic complications, including cardiovascular disease (CVD). There is currently no therapy for directly reducing concentrations of these compounds in humans.

Among the primary causes of CVD in T2DM is the nonenzymatic formation of advanced glycation endproducts (AGEs) from reactive dicarbonyl sugars such as methylglyoxal (MG) and 3-deoxyglucosone (3DG). MG irreversibly adducts arginine residues in proteins which subsequently alter the charge status (see, FIG. *2) creating oxidative stress and an environment in which atherosclerosis is promoted. However, the effect of AGEs on thrombosis has not been elucidated, and the ultimate mechanisms through which hyperglycemia promote vascular disease are not fully understood.

The process of thrombosis is normally counteracted by the process of fibrinolysis to maintain hemostasis. Plasminogen (Pg), a zymogen released from the liver, is converted into plasmin by the enzyme tissue plasminogen activator (tPA) via cleavage between arginine-561 (R561) and valine-562 (V562). Plasmin is the active enzyme which degrades the fibrin backbone of a clot. In vivo fibrinolysis may be impaired as a result of MG modification of Pg. Due to the steric and electrostatic changes that MG adduction causes, experiments were conducted that hypothesized that functional impairment of normal hemostasis may be due to adduction of critical arginine(s) on Pg.

Accordingly, provided herein is a technology relating to the interaction between metformin and methylglyoxal. In particular, the present invention provides methods, compositions, and related uses for the interaction between metformin and methylglyoxal. In addition, the present invention provides methods for detecting the efficacy of metformin therapy.

The present invention is not limited to particular methods or techniques for detecting such biomarkers in a biological sample (e.g., IMZ as indicative of a MF/MG product; MG modified plasminogen (Pg) product as indicative of impaired fibrinolysis; o-phenylenediamine derivatized MG (2MQ) product as indicative of the presence of MG).

Methods of obtaining test samples are well known to those of skill in the art and include, but are not limited to, aspirations, tissue sections, drawing of blood or other fluids, surgical or needle biopsies, and the like. The test sample may be obtained from an individual or patient diagnosed as having diabetes. The test sample may be a cell-containing liquid or a tissue. Samples may include, but are not limited to, urine, amniotic fluid, biopsies, blood, blood cells, bone marrow, fine needle biopsy samples, peritoneal fluid, amniotic fluid, plasma, pleural fluid, saliva, semen, serum, tissue or tissue homogenates, frozen or paraffin sections of tissue. Samples may also be processed, such as sectioning of tissues, fractionation, purification, or cellular organelle separation.

Any suitable method may be used to detect IMZ as indicative of a MF/MG product, MG modified plasminogen (Pg) product as indicative of impaired fibrinolysis, and/or o-phenylenediamine derivatized MG (2MQ) product as indicative of the presence of MG.

Several methods for detection of proteins are well known in the art. Detection of the proteins could be by resolution of the proteins by SDS polyacrylamide gel electrophoresis (SDS PAGE), followed by staining the proteins with suitable stain for example, Coomassie Blue. Such markers can be differentiated from each other and also from other proteins by Western blot analysis using specific antibodies. Methods of Western blot are well known in the art and described for example in W. Burnette W. N. Anal. Biochem. 1981; 112 (2): 195-203.

Alternatively, flow cytometry may be applied to detect the markers. Antibodies specific for the markers can be coupled to beads and can be used in the flow cytometry analysis.

In some embodiments, protein microarrays may be applied to identify the various markers. Methods of protein arrays are well known in the art. In one example, antibodies specific for each protein may be immobilized on the solid surface such as glass or nylon membrane. The proteins can then be immobilized on the solid surface through the binding of the specific antibodies. Antibodies may be applied that bind specifically to a second epitope (e.g., an epitope common to the marker) of the proteins. The first antibody/protein/second antibody complex can then be detected using a detectably labeled secondary antibody. The detectable label can be detected as discussed for polynucleotides.

Various procedures known in the art may be used for the production of antibodies to epitopes of the markers that may be used to distinguish among the protein variants. Such antibodies include but are not limited to polyclonal, monoclonal, chimeric, single chain, Fab fragments and fragments produced by a Fab expression library.

Antibodies may be radioactively labeled allowing one to follow their location and distribution in the body after injection. Radioactivity tagged antibodies may be used as a non-invasive diagnostic tool for imaging de novo cells.

Mass spectrometry is a particularly powerful methodology to resolve different forms of a protein because the different forms typically have different masses that can be resolved by mass spectrometry. Accordingly, if one form of a protein is a superior biomarker for a disease than another form of the biomarker, mass spectrometry may be able to specifically detect and measure the useful form where traditional immunoassay fails to distinguish the forms and fails to specifically detect to useful biomarker.

One useful methodology for detecting a specific marker as described combines mass spectrometry with immunoassay. First, a biospecific capture reagent (e.g., an antibody, aptamer or Affibody that recognizes the biomarker and other forms of it) is used to capture the biomarker of interest. Preferably, the biospecific capture reagent is bound to a solid phase, such as a bead, a plate, a membrane or an array. After unbound materials are washed away, the captured analytes are detected and/or measured by mass spectrometry. In some embodiments, such methods also permit capture of protein interactors, if present, that are bound to the proteins or that are otherwise recognized by antibodies and that, themselves, can be biomarkers. Various forms of mass spectrometry are useful for detecting the protein forms, including laser desorption approaches, such as traditional MALDI or SELDI, and electrospray ionization.

In some embodiments, a biomarker of this invention is detected by mass spectrometry, a method that employs a mass spectrometer to detect gas phase ions. Examples of mass spectrometers are time-of-flight, magnetic sector, quadrupole filter, ion trap, ion cyclotron resonance, electrostatic sector analyzer and hybrids of these.

In some embodiments, the mass spectrometer is a laser desorption/ionization mass spectrometer. In laser desorption/ionization mass spectrometry, the analytes are placed on the surface of a mass spectrometry probe, a device adapted to engage a probe interface of the mass spectrometer and to present an analyte to ionizing energy for ionization and introduction into a mass spectrometer. A laser desorption mass spectrometer employs laser energy, typically from an ultraviolet laser, but also from an infrared laser, to desorb analytes from a surface, to volatilize and ionize them and make them available to the ion optics of the mass spectrometer.

In some embodiments, the mass spectrometric technique for use is “Surface Enhanced Laser Desorption and Ionization” or “SELDI,” as described, for example, in U.S. Pat. Nos. 5,719,060 and 6,225,047; each herein incorporated by reference in its entirety. This refers to a method of desorption/ionization gas phase ion spectrometry (e.g. mass spectrometry) in which an analyte (e.g., one or more of the biomarkers of the present invention) is captured on the surface of a SELDI mass spectrometry probe.

In some embodiments, a sample is analyzed by means of a biochip. Biochips generally comprise solid substrates and have a generally planar surface, to which a capture reagent (also called an adsorbent or affinity reagent) is attached. Frequently, the surface of a biochip comprises a plurality of addressable locations, each of which has the capture reagent bound there. For example, in some embodiments, the present invention provides biochips having attached thereon one or more capture reagents specific for a marker of the present invention.

Protein biochips are biochips adapted for the capture of polypeptides (e.g., a marker as described herein). Many protein biochips are described in the art. These include, for example, protein biochips produced by Ciphergen Biosystems, Inc. (Fremont, Calif.), Zyomyx (Hayward, Calif.), Invitrogen (Carlsbad, Calif.), Biacore (Uppsala, Sweden) and Procognia (Berkshire, UK). Examples of such protein biochips are described in the following patents or published patent applications: U.S. Pat. Nos. 6,225,047, 6,537,749, 6,329,209, and 5,242,828, and PCT International Publication Nos. WO 00/56934, and WO 03/048768; each herein incorporated by reference in its entirety.

In certain embodiments, the present invention provides methods for managing a subject's treatment based on the status (e.g., presence or absence of a marker). Such management includes the actions of the physician or clinician subsequent to determining the marker status. For example, if a physician determines the presence of 2MQ in a biological sample obtained from the subject, treatment options directed at diabetic complications can be undertaken. If a physician determines the presence of IMZ following treatment with metformin, treatment based upon the metformin can be continued, modified, or cancelled. If a physician determines the presence of a MG/Pg product, treatment directed towards improving fibrinolysis can be undertaken.

In another aspect, the present invention provides compositions of matter based on the biomarkers of this invention. For example, in one embodiment, the present invention provides a biomarker of this invention in purified form. Purified biomarkers have utility as antigens to raise antibodies. Purified biomarkers also have utility as standards in assay procedures. As used herein, a “purified biomarker” is a biomarker that has been isolated from other proteins and peptides, and/or other material from the biological sample in which the biomarker is found.

Biomarkers may be purified using any method known in the art, including, but not limited to, mechanical separation (e.g., centrifugation), ammonium sulphate precipitation, dialysis (including size-exclusion dialysis), size-exclusion chromatography, affinity chromatography, anion-exchange chromatography, cation-exchange chromatography, and methal-chelate chromatography. Such methods may be performed at any appropriate scale, for example, in a chromatography column, or on a biochip.

In another embodiment, the present invention provides a biospecific capture reagent, optionally in purified form, that specifically binds a biomarker of this invention. In one embodiment, the biospecific capture reagent is an antibody. Such compositions are useful for detecting the biomarker in a detection assay, e.g., for diagnostics.

In another embodiment, this invention provides an article comprising a biospecific capture reagent that binds a biomarker of this invention, wherein the reagent is bound to a solid phase. For example, this invention contemplates a device comprising bead, chip, membrane, monolith or microtiter plate derivatized with the biospecific capture reagent. Such articles are useful in biomarker detection assays.

In another aspect the present invention provides a composition comprising a biospecific capture reagent, such as an antibody, bound to a biomarker of this invention, the composition optionally being in purified form. Such compositions are useful for purifying the biomarker or in assays for detecting the biomarker.

In another embodiment, this invention provides an article comprising a solid substrate to which is attached an adsorbent, e.g., a chromatographic adsorbent or a biospecific capture reagent, to which is further bound a biomarker of this invention.

In some embodiments, the methods disclosed herein are useful in monitoring the treatment of a specific condition (e.g., fibrinolysis) (e.g., onset of diabetic complications) (e.g., monitoring efficacy of metformin treatment). For example, in some embodiments, the methods may be performed immediately before, during and/or after a treatment to monitor treatment success. In some embodiments, the methods are performed at intervals on disease free patients to ensure treatment success.

The present invention also provides a variety of computer-related embodiments. Specifically, in some embodiments the invention provides computer programming for analyzing and comparing a pattern of marker detection results in a sample obtained from a subject to, for example, a library of such marker patterns known to be indicative of the presence or absence of a particular condition, or a particular stage or prognosis of the condition.

In some embodiments, the present invention provides computer programming for analyzing and comparing a first and a second pattern of marker detection results from a sample taken at least two different time points. In some embodiments, the first pattern may be indicative of a non-disease condition and/or low risk condition and/or progression from a pre-condition to a condition. In such embodiments, the comparing provides for monitoring of the progression of the condition from the first time point to the second time point.

In yet another embodiment, the invention provides computer programming for analyzing and comparing a pattern of marker detection results from a sample to a library of marker patterns known to be indicative of the presence or absence of such a condition, wherein the comparing provides, for example, a differential diagnosis between an aggressive form and a less aggressive form (e.g., the marker pattern provides for staging and/or grading of the condition).

In certain embodiments, the present invention provides methods for obtaining a subject's risk profile for developing a condition (e.g., diabetic complication, fibrinolysis) or having an aggressive form of such a condition. In some embodiments, such methods involve obtaining a biological sample (urine sample, blood sample) from a subject (e.g., a human at risk for developing a diabetic complication and/or fibrinolysis; a human undergoing a routine physical examination, or a human diagnosed with such a condition), detecting the presence or absence of a marker described herein in the sample, and generating a risk profile for developing such a condition or progressing to an aggressive form of such a condition. For example, in some embodiments, a generated profile will change depending upon specific markers and detected as present or absent or at defined threshold levels. The present invention is not limited to a particular manner of generating the risk profile. In some embodiments, a processor (e.g., computer) is used to generate such a risk profile. In some embodiments, the processor uses an algorithm (e.g., software) specific for interpreting the presence and absence of specific markers as determined with the methods of the present invention. In some embodiments, the presence and absence of specific markers described herein as determined with the methods of the present invention are imputed into such an algorithm, and the risk profile is reported based upon a comparison of such input with established norms (e.g., established norm for pre-condition, established norm for various risk levels for developing such a condition, established norm for subjects diagnosed with various stages of such a condition). In some embodiments, the risk profile indicates a subject's risk for developing such a condition or a subject's risk for re-developing such a condition. In some embodiments, the risk profile indicates a subject to be, for example, a very low, a low, a moderate, a high, and a very high chance of developing or re-developing such a condition or having a poor prognosis (e.g., likelihood of long term survival) from such a condition. In some embodiments, a health care provider (e.g., an endocrinologist) will use such a risk profile in determining a course of treatment or intervention.

The present inventions also contemplate diagnostic systems in kit form. A diagnostic system of the present inventions may include a kit which contains, in an amount sufficient for at least one assay, any of the agents for detecting the markers of the present invention (e.g., antibodies against such markers) in a packaging material. Typically, the kits will also include instructions recorded in a tangible form (e.g., contained on paper or an electronic medium) for using the agents in a detection assay in a test sample.

The present invention further relates to methods of treating, ameliorating, or preventing disorders in a patient, such as type 2 diabetes and/or complications related to type 2 diabetes, comprising administering to the patient a metformin and methylglyoxal-derived imidazolinone compound

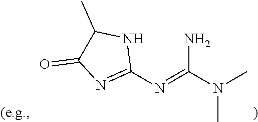

or a derivative or mimetic thereof and optionally additional agent(s).

In some embodiments, the compositions and methods of the present invention are used to treat diseased cells, tissues, organs, or pathological conditions and/or disease states in an animal (e.g., a mammalian patient including, but not limited to, humans and veterinary animals) characterized as having or at risk for having type 2 diabetes and/or complications related to type 2 diabetes.

Some embodiments of the present invention provide methods for administering an effective amount of a metformin and methylglyoxal-derived imidazolinone compound

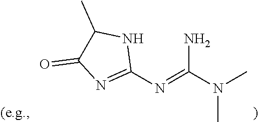

or a derivative or mimetic thereof and at least one additional therapeutic agent (including, but not limited to, agents known for treating type 2 diabetes and/or complications related to type 2 diabetes). A number of suitable agents known for treating type 2 diabetes and/or complications related to type 2 diabetes are contemplated for use in the methods of the present invention. Examples of additional agents known for treating type 2 diabetes and/or complications related to type 2 diabetes include, but are not limited to, biguanides (e.g., metformin), sulfonylureas (e.g., glyburide, glipizide, and glimepiride), meglitinide derivatives (e.g., repaglinide, nateglinide), alpha-gluocsidase inhibitors (e.g., acarbose, miglitol), thiazolidinediones (e.g., pioglitazone, rosiglitazone), glucagonlike peptide-1 agonists (e.g., exenatide, liraglutide, albiglutide, dulaglutide), dipeptidyl peptidase IV inhibitors (e.g., sitagliptin, saxagliptin, linagliptin, alogliptin), amylinomimetics (e.g., pramlintide), sodium-glucose transporter-2 inhibitors (e.g., canagliflozin, dapagliflozin, empagliflozin), bile acid sequestrants (e.g., colesevelam), rapid acting insulins (e.g., insulin aspart, insulin glulisine, insulin lispro, insulin inhaled), short acting insulins (e.g., regular insulin), intermediate acting insulins (e.g., insulin NPH), long acting insulins (e.g., insulin detemir, insulin glargine, insulin degludec), antiparkinson agents/dopamine agonists (e.g., bromocriptine), angiotensin receptor blockers (e.g., captopril, enalapril, lisinopril, losartan, valsartan, irbesartan), beta-adrenergic blocking agents (e.g., metoprolol, atenolol, labetalol), calcium channel blockers (e.g., diltiazem, verapamil, nifedipine, amlodipine), diuretics (e.g., furosemide, hydrochlorothiazide, bumetanide), direct renin inhibitors (e.g., aliskiren), sodium-glucose cotransporter 2 inhibitors (e.g., canagliflozin), nonsteroidal anti-inflammatory drugs (e.g., ibuprofen, naproxen), analgesics (e.g., capsaicin cream), anticonvulsants (e.g., gabapentin, carbamazepine, pregabalin, phenytoin), antidepressants (e.g., amitriptyline, imipramine, nortriptyline, duloxetine, citalopram, paroxetine, desipramine), antiarrhythmic agents (e.g., lidocaine anesthetic), prokinetic agents (e.g., erythromycin, cisapride, metoclopramide), synthetic adrenocortical steroids (e.g., fludrocortisone acetate), cholinergic agents (e.g., bethanechol hydrochloride), laxatives (e.g., polyethylene glycol solution), corticosteroids (e.g., triamcinolone acetonide), ophthalmics (e.g., ranibizumab, aflibercept intravitreal), HMG-CoA reductase inhibitors (e.g., pravastatin, simvastatin, lovastatin, fluvastatin, atorvastatin, rosuvastatin, pitavastatin), and fibric acid derivatives. Numerous other examples of agents known for treating type 2 diabetes and/or complications related to type 2 diabetes suitable for co-administration with the disclosed compounds are known to those skilled in the art.

In some embodiments of the present invention, a metformin and methylglyoxal-derived imidazolinone compound

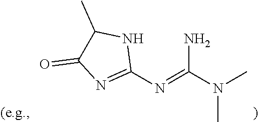

or a derivative or mimetic thereof and one or more additional agents (e.g., agents known for treating treating type 2 diabetes and/or complications related to type 2 diabetes) are administered to an animal under one or more of the following conditions: at different periodicities, at different durations, at different concentrations, by different administration routes, etc. In some embodiments, the metformin and methylglyoxal-derived imidazolinone compound

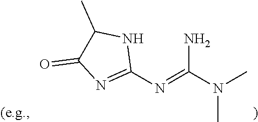

or a derivative or mimetic thereof is administered prior to the additional agent, e.g., 0.5, 1, 2, 3, 4, 5, 10, 12, or 18 hours, 1, 2, 3, 4, 5, or 6 days, or 1, 2, 3, or 4 weeks prior to the administration of the additional agent. In some embodiments, the compound is administered after the additional agent, e.g., 0.5, 1, 2, 3, 4, 5, 10, 12, or 18 hours, 1, 2, 3, 4, 5, or 6 days, or 1, 2, 3, or 4 weeks after the administration of the additional agent. In some embodiments, the compound and the additional agent are administered concurrently but on different schedules, e.g., the compound is administered daily while the additional agent is administered once a week, once every two weeks, once every three weeks, or once every four weeks. In other embodiments, the compound is administered once a week while the additional agent is administered daily, once a week, once every two weeks, once every three weeks, or once every four weeks.

Compositions within the scope of this invention include all compositions wherein the compounds of the present invention are contained in an amount which is effective to achieve its intended purpose. While individual needs vary, determination of optimal ranges of effective amounts of each component is within the skill of the art. Typically, the compounds may be administered to mammals, e.g. humans, orally at a dose of 0.0025 to 50 mg/kg, or an equivalent amount of the pharmaceutically acceptable salt thereof, per day of the body weight of the mammal being treated for disorders responsive to induction of apoptosis. In one embodiment, about 0.01 to about 25 mg/kg is orally administered to treat, ameliorate, or prevent such disorders. For intramuscular injection, the dose is generally about one-half of the oral dose. For example, a suitable intramuscular dose would be about 0.0025 to about 25 mg/kg, or from about 0.01 to about 5 mg/kg.

The unit oral dose may comprise from about 0.01 to about 1000 mg, for example, about 0.1 to about 100 mg of the compound. The unit dose may be administered one or more times daily as one or more tablets or capsules each containing from about 0.1 to about 10 mg, conveniently about 0.25 to 50 mg of the compound or its solvates.

In a topical formulation, the compound may be present at a concentration of about 0.01 to 100 mg per gram of carrier. In a one embodiment, the compound is present at a concentration of about 0.07-1.0 mg/ml, for example, about 0.1-0.5 mg/ml, and in one embodiment, about 0.4 mg/ml.

In addition to administering the compound as a raw chemical, the compounds of the invention may be administered as part of a pharmaceutical preparation containing suitable pharmaceutically acceptable carriers comprising excipients and auxiliaries which facilitate processing of the compounds into preparations which can be used pharmaceutically. The preparations, particularly those preparations which can be administered orally or topically and which can be used for one type of administration, such as tablets, dragees, slow release lozenges and capsules, mouth rinses and mouth washes, gels, liquid suspensions, hair rinses, hair gels, shampoos and also preparations which can be administered rectally, such as suppositories, as well as suitable solutions for administration by intravenous infusion, injection, topically or orally, contain from about 0.01 to 99 percent, in one embodiment from about 0.25 to 75 percent of active compound(s), together with the excipient.

The pharmaceutical compositions of the invention may be administered to any patient which may experience the beneficial effects of the compounds of the invention. Foremost among such patients are mammals, e.g., humans, although the invention is not intended to be so limited. Other patients include veterinary animals (cows, sheep, pigs, horses, dogs, cats and the like).

The compounds and pharmaceutical compositions thereof may be administered by any means that achieve their intended purpose. For example, administration may be by parenteral, subcutaneous, intravenous, intramuscular, intraperitoneal, transdermal, buccal, intrathecal, intracranial, intranasal or topical routes. Alternatively, or concurrently, administration may be by the oral route. The dosage administered will be dependent upon the age, health, and weight of the recipient, kind of concurrent treatment, if any, frequency of treatment, and the nature of the effect desired.

The pharmaceutical preparations of the present invention are manufactured in a manner which is itself known, for example, by means of conventional mixing, granulating, dragee-making, dissolving, or lyophilizing processes. Thus, pharmaceutical preparations for oral use can be obtained by combining the active compounds with solid excipients, optionally grinding the resulting mixture and processing the mixture of granules, after adding suitable auxiliaries, if desired or necessary, to obtain tablets or dragee cores.

Suitable excipients are, in particular, fillers such as saccharides, for example lactose or sucrose, mannitol or sorbitol, cellulose preparations and/or calcium phosphates, for example tricalcium phosphate or calcium hydrogen phosphate, as well as binders such as starch paste, using, for example, maize starch, wheat starch, rice starch, potato starch, gelatin, tragacanth, methyl cellulose, hydroxypropylmethylcellulose, sodium carboxymethylcellulose, and/or polyvinyl pyrrolidone. If desired, disintegrating agents may be added such as the above-mentioned starches and also carboxymethyl-starch, cross-linked polyvinyl pyrrolidone, agar, or alginic acid or a salt thereof, such as sodium alginate. Auxiliaries are, above all, flow-regulating agents and lubricants, for example, silica, talc, stearic acid or salts thereof, such as magnesium stearate or calcium stearate, and/or polyethylene glycol. Dragee cores are provided with suitable coatings which, if desired, are resistant to gastric juices. For this purpose, concentrated saccharide solutions may be used, which may optionally contain gum arabic, talc, polyvinyl pyrrolidone, polyethylene glycol and/or titanium dioxide, lacquer solutions and suitable organic solvents or solvent mixtures. In order to produce coatings resistant to gastric juices, solutions of suitable cellulose preparations such as acetylcellulose phthalate or hydroxypropylmethyl-cellulose phthalate, are used. Dye stuffs or pigments may be added to the tablets or dragee coatings, for example, for identification or in order to characterize combinations of active compound doses.

Other pharmaceutical preparations which can be used orally include push-fit capsules made of gelatin, as well as soft, sealed capsules made of gelatin and a plasticizer such as glycerol or sorbitol. The push-fit capsules can contain the active compounds in the form of granules which may be mixed with fillers such as lactose, binders such as starches, and/or lubricants such as talc or magnesium stearate and, optionally, stabilizers. In soft capsules, the active compounds are in one embodiment dissolved or suspended in suitable liquids, such as fatty oils, or liquid paraffin. In addition, stabilizers may be added.

Possible pharmaceutical preparations which can be used rectally include, for example, suppositories, which consist of a combination of one or more of the active compounds with a suppository base. Suitable suppository bases are, for example, natural or synthetic triglycerides, or paraffin hydrocarbons. In addition, it is also possible to use gelatin rectal capsules which consist of a combination of the active compounds with a base. Possible base materials include, for example, liquid triglycerides, polyethylene glycols, or paraffin hydrocarbons.

Suitable formulations for parenteral administration include aqueous solutions of the active compounds in water-soluble form, for example, water-soluble salts and alkaline solutions. In addition, suspensions of the active compounds as appropriate oily injection suspensions may be administered. Suitable lipophilic solvents or vehicles include fatty oils, for example, sesame oil, or synthetic fatty acid esters, for example, ethyl oleate or triglycerides or polyethylene glycol-400. Aqueous injection suspensions may contain substances which increase the viscosity of the suspension include, for example, sodium carboxymethyl cellulose, sorbitol, and/or dextran. Optionally, the suspension may also contain stabilizers.

The topical compositions of this invention are formulated in one embodiment as oils, creams, lotions, ointments and the like by choice of appropriate carriers. Suitable carriers include vegetable or mineral oils, white petrolatum (white soft paraffin), branched chain fats or oils, animal fats and high molecular weight alcohol (greater than C₁₂). The carriers may be those in which the active ingredient is soluble. Emulsifiers, stabilizers, humectants and antioxidants may also be included as well as agents imparting color or fragrance, if desired. Additionally, transdermal penetration enhancers can be employed in these topical formulations. Examples of such enhancers can be found in U.S. Pat. Nos. 3,989,816 and 4,444,762.

Ointments may be formulated by mixing a solution of the active ingredient in a vegetable oil such as almond oil with warm soft paraffin and allowing the mixture to cool. A typical example of such an ointment is one which includes about 30% almond oil and about 70% white soft paraffin by weight. Lotions may be conveniently prepared by dissolving the active ingredient, in a suitable high molecular weight alcohol such as propylene glycol or polyethylene glycol.

The invention is further illustrated by the following examples. The examples are provided for illustrative purposes only. They are not to be construed as limiting the scope or content of the invention in any way.

### EXAMPLES

The following examples are illustrative, but not limiting, of the compounds, compositions, and methods of the present invention. Other suitable modifications and adaptations of the variety of conditions and parameters normally encountered in clinical therapy and which are obvious to those skilled in the art are within the spirit and scope of the invention.

### Example 1

This example describes sensitive sites of glycation of the fibrinolytic system protein plasminogen and implications in the progression of type 2 diabetes cardiovascular complications.

Experiments were conducted to demonstrate the functional significance of MG modification of Pg was demonstrated via activation alterations by the endogenous activators tPA, urokinase plasminogen activator (uPA), and exogenous thrombolytic drug streptokinase (STK). Two-dimensional gel electrophoresis and LC-MS/MS on an LTQ Orbitrap Velos mass spectrometer were undertaken to determine sites of early adduction. Readily MG-adducted sites of significance identified include R43 in the PAp domain of the protein, and R504 and R530 of KR5 of Pg. A large number of other MG-modified residues and peptides were also detected.

### Example 1 Materials and Methods

Sequencing grade trypsin was purchased from Promega (Fitchburg, Wis.). 40% MG solution, thrombin and streptokinase were purchased from Sigma-Aldrich. Glu-Pg, uPA, tPA, fibrinogen, and chromogenic substrate for Pn were purchased from Molecular Innovations (Novi, Mich.).

In Vitro Modification of Pg with MG.

10 μg Pg was incubated with MG (50-500 μM) in tris-buffered saline (TBS) for 24 hours at 37° C. Following modification, sample volumes were increased to 150 μL and placed in 5 kDa spin filters to reduce volume and remove excess MG from solution.

Chromogenic Activity Assay.

96-well plate was incubated with 200 μL of 260 nM fibrinogen in TBS for 40 min at room temperature. Fibrinogen solution was removed and replaced with 250 μL TBS containing 3% bovine serum albumin and 0.01% TWEEN for 90 minutes at 37° C. Blocking solution was removed and the plate was washed twice with solution of 50 mM Tris-HCl, 110 mM NaCl and 0.01% TWEEN (pH 7.4). 5 units/mL of thrombin and 5 mM CaCl₂ in 50 mM Tris-HCl, 110 mM NaCl (pH 7.4) was added to the wells for 45 min. A high salt wash (1 M NaCl, 50 mM Tris-HCl, pH 7.4) followed by a TBS-TWEEN wash completed plate prep.

Into each well 198 μL of 10 mM HEPES/150 mM NaCl (pH 7.4) was added. Into each well, 2 μL of 25 mM chromogenic substrate for Pn (D-VLK-pNA) was added. To appropriate wells were added 2 μL of 20 μM Pg, either a 24-hour unmodified control or 24 hour 50-500 μM MG modified. Activators were added to respective wells to test activation of unmodified and modified Pg. 2 μL of each activator enzyme was utilized in appropriate wells for tPA (1.2 μM), uPA (5 μM) and STK (500 nM). Following enzyme addition, plate was immediately read on a SpectraMax M2 plate reader (Molecular Devices) every minute for 90 minutes at an absorbance of 405 nm.

Two Dimensional Gel Electrophoresis.

MG-modified (500 μM) and unmodified Pg control (same incubation conditions without MG) were added to 250 μL amount of rehydration buffer (6M urea, 2M thiourea, 2% CHAPS, 100 mM DTT, 0.5% 3-10 ampholytes) and 5 μL of 3-10 ampholytes (Bio-Rad). Sample rehydrated with a 13 cm 4-7 pH isoelectric focusing strip (GE Healthcare) overnight. Following rehydration, strips were placed in a Bio-Rad PROTEAN IEF Cell focusing instrument. Strips were first focused for 1 hour at 500 V, and then run at a maximum of 8,000V until the strips have reached a final total of 45,000 V/hrs at which point gels are held at 500V until removed. Focused strips were removed, reduced with DTT, cysteines carbamidomethylated with iodoacetamide, and run on a 4-20% gradient Criterion gel for 55 minutes at 200V. Gels were stained for stained according to manufacturer instructions with Imperial Blue Protein (ThermoScientific, Rockford, Ill.) and destained immediately with water. Gel bands were excised and trypsin digested.

Gel Excision, Cleanup and Digestion.

Coomassie stained gel-bands were excised and washed in ddH₂O for 15 min. H₂O was removed and bands were incubated in 50/50 acetonitrile (ACN):ddH₂O for 15 min. ACN:ddH₂O was removed and bands were incubated with ACN for 5 min. ACN was removed and gels were incubated with 100 mM ammonium bicarbonate (AMBIC). An equal volume of ACN was added to make 1:1 solution and was incubated for 15 min. The ACN/AMBIC solution was removed and remaining gel bands were dried by speed-vacuum. 10 mM dithiothreitol (DTT) was added to each band and incubated at 56° C. for 45 min. DTT was removed and sample brought to room temperature (RT). Iodoacetamide (55 mM) was added to each sample and incubated at RT for 30 min in dark. Iodoacetamide was removed and 100 mM AMBIC was added and bands were incubated for 5 min. An equal volume of ACN was added to make a 1:1 solution and incubated for 15 min. The ACN/AMBIC solution was removed and bands were dried by speed-vacuum. Bands were digested with trypsin (400 ng/band) in 50 mM AMBIC and incubated on ice for 45 min. Tryptic solution was removed and 50 mM AMBIC was added to each band and incubated overnight at 37° C. to complete digestion. Digests were acidified using 10% trifluoroacetic acid (TFA). Supernatant was saved. Band was covered with TFA:ACN (0.1%:60%) and sonicated at 20° C. water bath for 30 min. Supernatant was combined with previous fraction. Sampled were speed vacuumed to a final volume of 10 μL prior to LC-MS/MS analysis on an LTQ Orbitrap Velos mass spectrometer as described below.

Plasma Protein Fractionation and Modification.

Plasma (150 μl) from patients were diluted to 300 μl with TBS pH 7.4 and centrifuged through a 0.2 μm pore size spin filter to remove particulates. This sample was applied to an IgY-14 Seppro LC5 (Sigma Aldrich) affinity column using an Prominence UFLC instrument (Shimadzu) according to the manufacturer's instructions. The flow-through and bound fractions were collected separately, concentrated, and stored at −80° C. until analysis. Flow-through samples (30 μg total protein) were run on a 10% SDS-PAGE gel, coomassie stained, and bands corresponding to the Pg control (˜100 kDa) were excised from each sample lane and underwent digestion and cleanup prior to LTQ-Orbitrap mass spectrometry analysis.

Tandem Mass Spectrometry Coupled to Liquid Chromatography (LC-MS/MS).

LC-MS/MS analysis of in-gel trypsin digested-proteins (Shevchenko et al., 1996) was carried out using a LTQ Orbitrap Velos mass spectrometer (Thermo Fisher Scientific, San Jose, Calif.) equipped with an Advion nanomate ESI source (Advion, Ithaca, N.Y.), following ZipTip (Millipore, Billerica, Mass.) C18 sample clean-up according to the manufacturer's instructions. Peptides were eluted from a C18 precolumn (100-μm id×2 cm, Thermo Fisher Scientific) onto an analytical column (75-μm ID×10 cm, C18, Thermo Fisher Scientific) using a 5-20% gradient of solvent B (acetonitrile, 0.1% formic acid) over 65 minutes, followed by a 20-35% gradient of solvent B over 25 minutes, all at a flow rate of 400 nl/min. Solvent A consisted of water and 0.1% formic acid. Data dependent scanning was performed by the Xcalibur v 2.1.0 software (Andon et al., 2002) using a survey mass scan at 60,000 resolution in the Orbitrap analyzer scanning m/z 350-1600, followed by collision-induced dissociation (CID) tandem mass spectrometry (MS/MS) of the fourteen most intense ions in the linear ion trap analyzer. For human samples analyzed, an inclusion list was utilized that first preferentially allowed the ions corresponding to known modified peptides to undergo CID prior to that of the most intense ions. Precursor ions were selected by the monoisotopic precursor selection (MIPS) setting with selection or rejection of ions held to a +/−10 ppm window. Dynamic exclusion was set to place any selected m/z on an exclusion list for 45 seconds after a single MS/MS. All MS/MS samples were analyzed using Sequest (Thermo Fisher Scientific, San Jose, Calif., USA; version 1.3.0.339). Sequest was set up to search human proteins downloaded from UniProtKB on Aug. 6, 2013. Variable modifications considered during the search included methionine oxidation (15.995 Da), cysteine carbamidomethylation (57.021 Da), as well as two products of the adduction of arginine residues by MG, MG-H1 (54.011 Da) and MG-DH (72.021 Da). At the time of the search, the Human UniProt database contained 88,323 entries. Proteins were identified at 95% confidence with XCorr scores (Qian et al., 2005) as determined by a reversed database search using the Percolator algorithm (//per-colator.com) (Spivak et al., 2009). Identified modified peptides were considered with a q-value<0.01 (Kall et al., 2008).

### Example 1 Results

Activation Changes Due to MG Modification of Pg.

In order to assess a broader overall effect of MG modification on Pg activation into Pn, an assay utilizing a chromogenic substrate was performed. The chromogenic substrate (where from) is cleaved in the presence of Pn, releasing a compound that is detectable by measuring absorbance at 405 nm. Because no Pn was added to the samples, all of the Pn would need to come from Pg being converted by its activators. Three activators, tPA, uPA, and STK, were studied in their ability to generate Pn from modified and unmodified Pg.

The assay indicated that with all three enzymes, MG modification of Pg for 24 hours led to a delay in activation of the protein (FIG. *4). Activation by tPA was had a significantly detectable delay (p<0.05) by 30 minutes for 100 μM MG-modified Pg and 28 minutes for 500 μM MG-modified Pg. Activation by uPA wasn't nearly as significant, only showing a significant delay in the 500 μM MG-modified Pg detectable beginning at 37 minutes. STK seemed to be the most affected by modification, with a delay in activation detectable by 16 minutes for 100 μM MG-modified Pg and 12 minutes for 500 μM MG-modified Pg.

2D Gel Identification of Isoelectric Point Shift.

Glu-Pg has a published isoelectric point of 6.2 (Robbins and Summaria, 1976). The observed isoelectric point of the Pg unmodified control appeared to be at a pI of roughly 6.5-6.8 (FIG. *5A). Modification of arginine on proteins by MG primarily creates hydroimidazolone, a product which results in charge loss from arginine. This charge removal shifts the pI of individual arginines (11.15) lower, reducing the overall pI of the protein. As more arginine residues are modified, it would be expected that overall isoelectric point of the protein would shift towards a lower pH, with varying isoelectric points depending on which and how many residues have been modified on the molecule. Upon 24 hour modification with 500 uM MG, there was a clear shift towards the 4 pH side of the strip (FIG. (FIG. *5B). The observable stained protein has become much wider than the unmodified control, with multiple observable spots of concentrated protein within the larger smear. The point of the modified protein spot with the highest pI appears to be equal to lowest observable pI point on the unmodified gel which was focused under identical conditions side by side with the modified strip.

Identification of Sensitive Sites of MG Adduction on PLG.

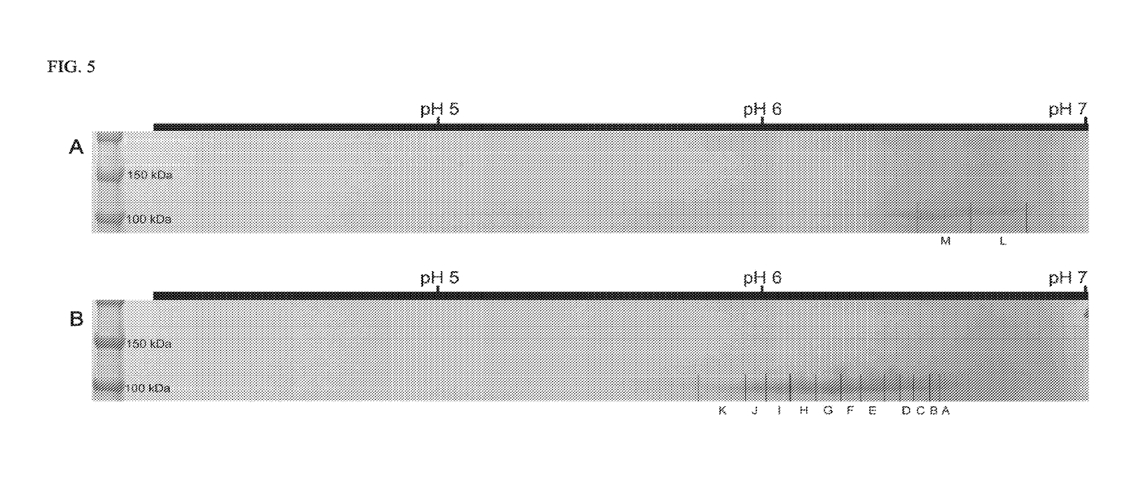

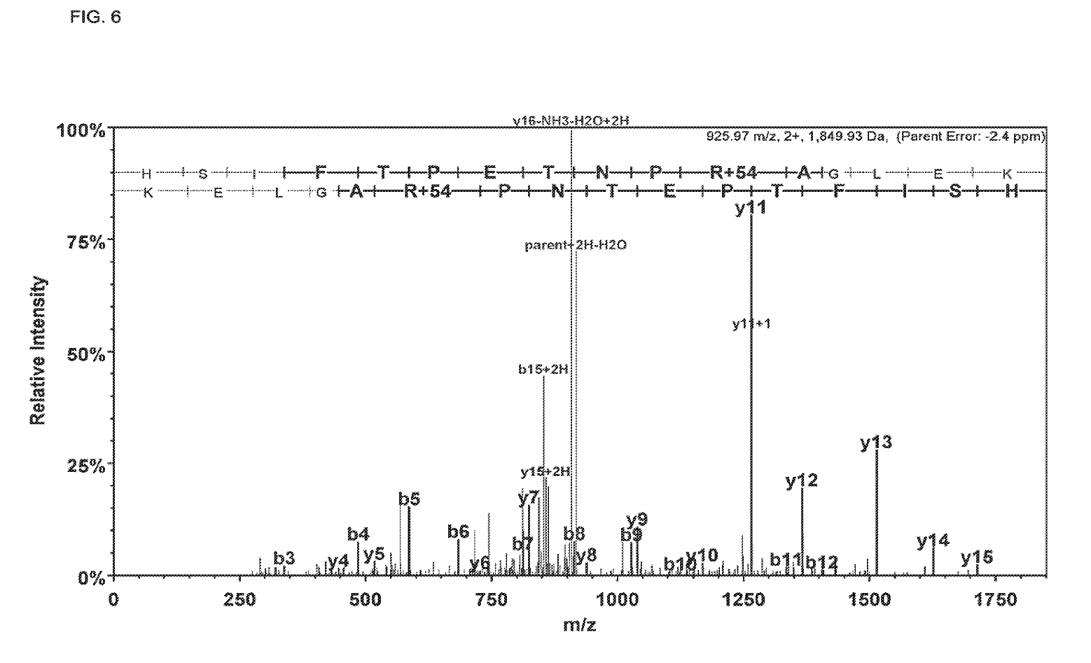

Protein spots from both modified and unmodified gels were excised as indicated in FIG. 5 and analyzed via LC-MS/MS to determine which arginine residues appeared to be among the early modifications detected. Due to the chromatographic resolution of the 1^{st} focusing dimension of the 2D PAGE process, the modified Pg protein smear was excised in numerous portions and divided in order to capture the visible protein spots. A large number of modified peptides (q-value<0.01) were detected as indicated in Table *1. The fewest modified peptides detected were in spot A, at a pI of roughly 6.7. The most modified peptides were detected in spot E, at a pI of 6.4. The amount of modified peptides detected increased from spot A to spot H in a somewhat linear fashion. Slight differences in amount of modified peptides detected may be due to variations in trypsin digestion, but the general trend noted above appears to be present. Spots I-K most likely had fewer modified peptides detected due to visibly lower proteins amounts in the excised bands as evidenced by the decreased staining intensity.

Six MG-modified arginine sites were detected in the first spot, spot A, with an addition of +54 daltons, representative of a MG-H1 modification. Three of these sites were detected in multiple peptides detected. R43, one the sites identified with multiple peptides, is located in the Pan-Apple (PAP) domain of the protein, which is critical for maintaining the closed-form conformation of Pg. Three other early modifications, R115, R223, and R312, are found on kringle domains 1, 2, and 3, respectively. The final two early modifications that were detected in spot A are R504 and R530, both of which were detected in multiple peptides. Both of these modifications are found on kringle domain 5 (KR5). The kringle domains in Pg are responsible for binding lysine residues. In regards to overall function, exposed lysine residues on fibrin result in recruitment of Pg via kringle-lysine binding. Five of the six early modifications detected occur within these kringle domains, and due to the altered size and charge due to adduction, could result in functional alterations of the protein.

Additional sites of early modification from a slightly lower pI in spot B include R61 and R70 from the PAP domain, R474 from KR5, and R712 from the serine protease domain of Pg. In addition to these four sites, all of the sites detected in spot A were also detected in spot B. The cleavage site arginine, R561, is identified as a site of adduction at spot D. As expected, no modifications were seen in spots L and M, both of which originated from the control gel.

Identification of Modifications in Human Samples.

Human plasma samples, both diabetic and non-diabetic, were selected from a larger cohort based on history of cardiovascular complications. Following SDS-PAGE focusing, the bands corresponding to Pg were cut, trypsin-digested and analyzed via LC-MS/MS on an LTQ Orbitrap Velos mass spectrometer. In many cases, a visible coomassie-stained band for Pg was not apparent, but all bands submitted showed the presence of Pg peptides, usually with greater than 70% protein coverage.

Nine of the 16 patient samples analyzed to date showed the presence of modified Pg peptides. While a number of the patients with modified peptides identified were diabetic, two of the patients were non-diabetic with CVD history, and another was both non-T2D and non-CVD. A large number of modified peptides, both with MG-DH and MG-H1 modifications were detected. Patient RS104, a patient with T2D with a history of myocardial infarction, provided the most modified peptides. R504, among the early modifications detected from the two-dimensional gel studies, was also observed in patient RS104 (FIG. *6). In addition to R504, common adducted peptides observed among patients included R134 and R582. Interestingly, R134 was only detected with MG-DH modification of +72. R582 modified peptides appeared in six of the patient samples, as both the +54 and +72 adduct. Of note is that neither R134 nor R582 were detected at all in the in vitro analysis performed, yet appear to be highly modified in humans.

### Example 1 Discussion

Pg and the fibrinolytic system play an important role in normal breakdown of the fibrin backbone of a clot. Recently findings have shown that Pg activation into Pn is altered in patients with T2DM, and accordingly, fibrinolysis is impaired (Ajjan et al., 2013). Improved glycemic control was able to reverse this impairment. While they identified two potential NE-fructosyl-lysine modifications that could be an underlying mechanism behind this impairment, further work is necessary to pinpoint the exact cause of this functional change.

The findings indicate that glycation of Pg by MG in vitro alters normal activation into Pn by all three major activator enzymes. Streptokinase in particular has previously been shown to have reduced effectiveness in treating myocardial infarction in patients that also had T2DM (Chowdhury et al., 2008). Glycation of Pg and resulting inhibition of activation by streptokinase could be an explanation for this phenomenon. This data, while intriguing, provides little insight into the underlying mechanism behind the altered function.

One of the important goals of this study was to determine not simply which arginine residues appeared to be readily modified by MG, but which were most sensitive to adduction and thus would be modified at the lowest concentrations. Though a high concentration of 500 μM MG was utilized in the two-dimensional gel studies, analyzing modified peptides based on isoelectric point allowed us to identify the least modified versions of the protein and thus capture the most sensitive sites. Six sites appeared to be the most sensitive to adduction, all of which appeared near sites critical for normal Pg function. Modification of R43, part of the Pan-Apple domain of the protein, is responsible for holding the protein in its closed form and preventing activation until Pg is bound to fibrin. It is possible that R43 modification could result in a gain of function, which holds Pg in its closed form conformation more strongly and prevents activation, or a loss of function, which would result in premature activation of Pg away from the clot site. The remainder of the early sites of modification were within kringle domains, which are responsible for binding exposed lysine residues on fibrin and incorporation of Pg into the clot. This binding to lysine residues triggers Pg to adopt an open form which is able to be activated. MG adduction to arginine residues in these regions could inhibit interactions between the kringle domains and fibrin lysines.

It has been suggested that kringle 5 in particular, where two readily adducted arginines (R504 and R530) were identified, interacts with the Pan-Apple domain and is the “Achilles heel” of Pg conformation change. Once the interaction between PAp and KR5 is severed, KR5 can bind to lysine residues on fibrin, leading to tertiary structure change that opens up the active site of the protein. Modifications in this region, which from this work, appear to be among the most prevalent modifications, and could drastically alter the functionality of the protein.

The recent published work studying reduced Pg activation due to glycation was unable to identify any hydroimidazolone or dihydroxyimidazolidine modifications in any human subjects tested. It is possible that due to experimental design flaws, certain modifications were excluded. If indeed lysine binding is inhibited by MG modification on arginine residues in the kringle domains of Pg, then utilization of a lysine sepharose column to purify out Pg, as the researchers did, would exclude modified Pg molecules with a reduced affinity for the column. A process which includes all possible Pg proteins is necessary to determine definitively that MG adducts are not present on arginine residues in human samples.

The process of utilizing a depletion column, which allows us to decrease the complexity of the sample while not excluding any Pg protein, allowed for us to detect MG-H1 and MG-DH on Pg peptides in nine of the sixteen patients. While R504 appeared to be a site of interest in humans, which corroborates data presented that it is an early and sensitive site of adduction, other modifications detected were a bit more puzzling. R504 modification could alter fibrin-lysine binding by Kringle 5, due to its location on the protein. R134 and R582 appeared in a large number of patients, yet were completely undetected in the in vitro studies performed. R134 is located in kringle 1 of the protein while R582 is located in the serine-protease region of Pg. It is possible that altered pH or in vivo conditions exposed these sites to modification that simply aren't easily reproducible in vitro. Salt is known to be necessary to hold Pg in its closed-form conformation, so it is possible that too low of salt was utilized in the two-dimensional gel studies and thus these sites were unable to be identified. While both of these modifications are in critical domains on the protein, the result of their modification on overall activation is unknown. It is possible that R582 modification could have a direct affect on the nearby R561-V562 cleavage site. Further molecular modeling studies are needed to assess potential consequences of these modifications. Previous work had identified R561 as a potential site of interest, but little modification of R561 was detected in the patients studied and most likely only plays a small role in the altered Pg activity observed in T2D patients.

In addition to the specific sites identified as described above, it is noteworthy to also discuss patient types. Both patients with and without T2D, and with and without CVD, were identified with MG-modifications on Pg peptides. While overall, it appeared as though T2D patients had more modifications, non-T2D patients were affected as well. MG is present in all humans, so it is expected there may be a baseline amount of modification present.

These results demonstrate that it is clear that MG-modification of arginine residues cannot be excluded as a potential mechanism behind overall inhibition of Pg function, particularly in type 2 diabetes. In particular, sites R43, R134, R504, R530, and R582 bear further study in order to determine what role modification of these residues may have in overall Pg function and activation.

Table 2 presents human sites of Pg modification.

### Table 1—Sensitive Sites of MG-Pg Adduction

Sites of modification observed in spots from 2D-gel spot excision and LTQ-Orbitrap analysis, sorted by spot in which peptide was first observed. In total, 23 single-modified peptides were observed, and three double-modified peptides were observed. Sites of initial modification are arginines 43, 115, 223, 312, 504, and 530. The only sites which appear in every spot are arginines 43, 312, 504, and 530. Cysteines were carbamidomethylated (+57). Bolded M indicates methionine oxidation observed. All modified peptides were considered with a q-value<0.01.

TABLE 1
Sensitive sites of adduction

| | | | | Spot of | |
|---|---|---|---|---|---|
| | | | | first | Additional |
| | | MG-H1 | MG-DH | peptide | spots with |
| Site | Peptide | (+54) | (+72) | ID | peptide IDs |
| R43 | (K)CEEDEEFTCRAFQYHSK(E) | X | X | A | B, C, D, E, F, |
| | | | | | G, H, I, J, K |
| R115 | (K)WSSTSPHRPR(F) | X | | A | B, D, E, F, H |
| R223 | (R)NPDRELRPWCFTTDPNKR(W) | X | X | A | B, C, D, E, F, |
| | | | | | H, I, J, K |
| R312 | (K)RAPWCHTTNSQVR(W) | X | X | A | B, C, D, E, F, |
| | | | | | G, H, I, J, K |
| R504 | (R)HSIFTPETNPRAGLEK(N) | X | X | A | B, C, D, E, F, |
| | | | | | G, H, I, J, K |
| R530 | (R)NPDGDVGGPWCYTTNPRK(L) | X | X | A | B, C, D, E, F, |
| | | | | | G, H, I, J, K |
| R61 | (K)EQQCVIMAENRK(S) | X | X | B | C, D, E, F, G, |
| | | | | | H, I, J, K |
| R70 | (R)MRDVVLFEK(K) | X | X | B | C, D, E, F, G, |
| | | | | | H, I, J, K |
| R474 | (R)GKRATTVTGTPCQDWAAQEPHR(H) | X | X | B | C, D, E, F, G, |
| | | | | | H, I, J, K |
| R712 | (K)VCNRYEFLNGR(V) | X | X | B | C, D, E, F, G, |
| | | | | | H, I, J, K |
| R789 | (R)FVTWIEGVMRNN(—) | X | X | C | D, E, F, G, H, |
| | | | | | I, J |
| R115/ | (K)WSSTSPHRPRFSPATHPSEGLEENYCR(N) | X | X | D | E, H, I, J |
| 117 | | | | | |
| R117 | (K)WSSTSPHRPRFSPATHPSEGLEENYCR(N) | X | X | D | E, H, I, J |
| R242 | (R)WELCDIPRCTTPPPSSGPTYQCLK(G) | X | X | D | E, F, H, I |
| R290 | (R)GNVAVTVSGHTCQHWSAQTPHTHNRTPENFPCK(N) | X | X | D | E, F, H, I |
| R324 | (K)RAPWCHTTNSQVRWEYCK(I) | X | X | D | E, H, I, J, K |
| R312/ | (K)RAPWCHTTNSQVRWEYCK(I) | X | X | D | E, F, H, I, J, |
| 324 | | | | | K |
| R493 | (K)RATTVTGTPCQDWAAQEPHRHSIFTPETNPR(A) | X | X | D | F, H, J |
| R561 | (K)KCPGRVVGGCVAHPHSWPWQVSLR(T) | X | X | D | E, I, J |
| R68 | (K)SSIIIRMR(D) | | X | E | K |
| R220 | (R)NPDRELRPWCFTTDPNKR(W) | X | X | E | F, H |
| R220/ | (R)NPDRELRPWCFTTDPNKR(W) | X | X | E | H, I, J, K |
| 223 | | | | | |
| R389 | (K)KCQSWSSMTPHRHQK(T) | X | X | E | F, H, I, J |
| R644 | (R)LFLEPTRK(D) | X | X | E | G |
| R767 | (K)DKYILQGVTSWGLGCARPNKPGVYVR(V) | X | X | E | |
| R265 | (K)GTGENYRGNVAVTVSGHTCQHWSAQTPHTHNR(T) | X | | H | |

### Table 2—Human Sites of MG-Pg Adduction

Sites of modification observed in human plasma samples following IgY-12 depletion, 1D-gel electrophoresis and excision, and LC-MS/MS analysis. Patient disease (with or without type 2 diabetes [T2D]) and cardiovascular disease (CVD) status was noted. All modified peptides were considered with a q-value<0.01. CABG: coronary artery bypass graft; MI: myocardial infarction.

TABLE 2
Human sites of modification

| | | | Sites | | | | |
|---|---|---|---|---|---|---|---|
| Sample | Disease | | Iden- | | MG-H1 | MG-DH | |
| ID | state | CVD | tified | Peptide | (+54) | (+72) | |
| CS45 | T2D | CABG | R134 | (R)FSPATHPSEGLEENYCRNPDNDPQGPWCYTTDPEKR | | x | |
| | | | | (Y) | | | |
| | | | R582 | (R)TRFGMHFCGGTLISPEWVLTAAHCLEK(S) | x | | |
| JA73 | T2D | CABG | R712 | (K)VCNRYEFLNGR(V) | | x | |
| RS104 | T2D | MI | R70 | (R)MRDVVLFEK(K) | x | | |
| | | | R117 | (K)WSSTSPHRPRFSPATHPSEGLEENYCR(N) | | x | |
| | | | R134 | (R)FSPATHPSEGLEENYCRNPDNDPQGPWCYTTDPEKR | | x | |
| | | | | (Y) | | | |
| | | | R223 | (R)NPDRELRPWCFTTDPNKR(W) | x | | |
| | | | R312 | (K)RAPWCHTTNSQVR(W) | x | | |
| | | | R324 | (K)RAPWCHTTNSQVRWEYCK(I) | x | | |
| | | | R504 | (R)HSIFTPETNPRAGLEK(N) | x | x | |
| | | | R561 | (K)KCPGRVVGGCVAHPHSWPWQVSLR(T) | | | |
| | | | R582 | (R)TRFGMHFCGGTLISPEWVLTAAHCLEK(S) | x | x | |
| | | | R644 | (K)VILGAHQEVNLEPHVQEIEVSRLFLEPTRK(D) | | x | |
| | | | R677 | (K)VIPACLPSPNYVVADRTECFITGWGETQGTFGAGLLK | x | x | |
| | | | | (E) | | | |
| | | | R712 | (K)VCNRYEFLNGR(V) | | x | |
| | | | R767 | (K)DKYILQGVTSWGLGCARPNKPGVYVR(V) | | x | x |
| | | | R779 | (R)VSRFVTWIEGVMR(N) | x | x | |
| | | | R789 | (R)FVTWIEGVMRNN(—) | x | x | |
| SS110 | T2D | MI | R134 | (R)FSPATHPSEGLEENYCRNPDNDPQGPWCYTTDPEKR | | x | |
| | | | | (Y) | | | |
| | | | R677 | (K)VIPACLPSPNYVVADRTECFITGWGETQGTFGAGLLK | | x | |
| | | | | (E) | | | |
| | | | R582 | (R)TRFGMHFCGGTLISPEWVLTAAHCLEK(S) | x | | |
| GB119 | T2D | CABG | R134 | (R)FSPATHPSEGLEENYCRNPDNDPQGPWCYTTDPEKR | | x | |
| | | | | (Y) | | | |
| | | | R677 | (K)VIPACLPSPNYVVADRTECFITGWGETQGTFGAGLLK | | x | |
| | | | | (E) | | | |
| | | | R582 | (R)TRFGMHFCGGTLISPEWVLTAAHCLEK(S) | x | | |
| NM130 | non- | MI | R134 | (R)FSPATHPSEGLEENYCRNPDNDPQGPWCYTTDPEKR | | x | |
| | T2D | | | (Y) | | | |
| | | | R504 | (R)HSIFTPETNPRAGLEK(N) | x | | |
| NY141 | T2D | CABG | R134 | (R)FSPATHPSEGLEENYCRNPDNDPQGPWCYTTDPEKR | | x | |
| | | | | (Y) | | | |
| | | | R582 | (R)TRFGMHFCGGTLISPEWVLTAAHCLEK(S) | x | | |
| | | | R677 | (K)VIPACLPSPNYVVADRTECFITGWGETQGTFGAGLLK | x | | |
| | | | | (E) | | | |
| | | | R712 | (K)VCNRYEFLNGR(V) | | x | |
| GG186 | non- | none | R312 | (K)RAPWCHTTNSQVR(W) | x | | |
| | T2D | | | | | | |
| | | | R504 | (R)HSIFTPETNPRAGLEK(N) | x | | |
| MO238 | non- | MI | R43 | (K)CEEDEEFTCRAFQYHSK(E) | x | | |
| | T2D | | | | | | |
| | | | R242 | (R)WELCDIPRCTTPPPSSGPTYQCLK(G) | | x | |
| | | | R530 | (R)NPDGDVGGPWCYTTNPRK(L) | x | | |
| | | | R582 | (R)TRFGMHFCGGTLISPEWVLTAAHCLEK(S) | x | x | |
| | | | R637 | (K)VILGAHQEVNLEPHVQEIEVSRLFLEPTR(K) | x | | |
| | | | R767 | (K)DKYILQGVTSWGLGCARPNKPGVYVR(V) | x | x | |
| | | | R789 | (R)FVTWIEGVMrNN(—) | x | | |

### Example 1 Bibliography

### References

Ajjan, R. A., Gamlen, T., Standeven, K. F., Mughal, S., Hess, K., Smith, K. A., Dunn, E. J., Anwar, M. M., Rabbani, N., Thornalley, P. J., Philippou, H., and Grant, P. J. (2013). Diabetes is associated with posttranslational modifications in plasminogen resulting in reduced plasmin generation and enzyme-specific activity. Blood. 122, 134-142.
Andon, N. L., Hollingworth, S., Koller, A., Greenland, A. J., Yates, J. R., 3rd, and Haynes, P. A. (2002). Proteomic characterization of wheat amyloplasts using identification of proteins by tandem mass spectrometry. Proteomics. 2, 1156-1168.
Castellino, F. J. and Ploplis, V. A. (2005). Structure and function of the plasminogen/plasmin system. Thromb. Haemost. 93, 647-654.
Chowdhury, M. A. R., Hossain, A. M., Dey, S. R., and Akhtaruzzaman, A. (2008). A comparative study on the effect of streptokinase between diabetic and non-diabetic myocardial infarction patients. Bangladesh Journal of Pharmacology. 3, 1-7.
Gonna, Y. and Lentzner, H. (2008). Multiple causes of death in old age. Aging Trends. (9), 1-9.
Gugliucci, A. (2003). A practical method to study functional impairment of proteins by glycation and effects of inhibitors using current coagulation/fibrinolysis reagent kits. Clin. Biochem. 36, 155-158.
Heron, M. (2012). Deaths: leading causes for 2008. Natl. Vital Stat. Rep. 60, 1-94.
Kall, L., Storey, J. D., MacCoss, M. J., and Noble, W. S. (2008). Posterior error probabilities and false discovery rates: two sides of the same coin. J. Proteome Res. 7, 40-44.
Kimzey, M. J., Yassine, H. N., Riepel, B. M., Tsaprailis, G., Monks, T. J., and Lau, S. S. (2011). New site(s) of methylglyoxal-modified human serum albumin, identified by multiple reaction monitoring, alter warfarin binding and prostaglandin metabolism. Chem. Biol. Interact. 192, 122-128.
Law, R. H., Caradoc-Davies, T., Cowieson, N., Horvath, A. J., Quek, A. J., Encarnacao, J. A., Steer, D., Cowan, A., Zhang, Q., Lu, B. G., Pike, R. N., Smith, A. I., Coughlin, P. B., and Whisstock, J. C. (2012). The X-ray crystal structure of full-length human plasminogen. Cell. Rep. 1, 185-190.
Lerant, I., Kolev, K., Gombas, J., and Machovich, R. (2000). Modulation of plasminogen activation and plasmin activity by methylglyoxal modification of the zymogen. Biochim. Biophys. Acta. 1480, 311-320.
Miyashita, C., Wenzel, E., and Heiden, M. (1988). Plasminogen: a brief introduction into its biochemistry and function. Haemostasis. 18 Suppl 1, 7-13.
Mustaffa, N., Ibrahim, S., Abdullah, W. Z., and Yusof, Z. (2011). Add-on rosiglitazone therapy improves plasminogen activity and high-density lipoprotein cholesterol in type 2 diabetes mellitus. Blood Coagul. Fibrinolysis. 22, 512-520.
Plutzky, J. (2011). Macrovascular effects and safety issues of therapies for type 2 diabetes. Am. J. Cardiol. 108, 25B-32B.

Qian, W. J., Liu, T., Monroe, M. E., Strittmatter, E. F., Jacobs, J. M., Kangas, L. J., Petritis, K., Camp, D. G., 2nd, and Smith, R. D. (2005). Probability-based evaluation of peptide and protein identifications from tandem mass spectrometry and SEQUEST analysis: the human proteome. J. Proteome Res. 4, 53-62.
Robbins, K. C. and Summaria, L. (1976). Plasminogen and plasmin. Methods Enzymol. 45, 257-273.
Shevchenko, A., Wilm, M., Vorm, O., and Mann, M. (1996). Mass spectrometric sequencing of proteins silver-stained polyacrylamide gels. Anal. Chem. 68, 850-858.
Spivak, M., Weston, J., Bottou, L., Kall, L., and Noble, W. S. (2009). Improvements to the percolator algorithm for Peptide identification from shotgun proteomics data sets. J. Proteome Res. 8, 3737-3745.

### Example 2

This example demonstrates that a cyclized imidazolinone derivative is the predominant methylglyoxal scavenging product from the anti-hyperglycemic drug metformin.

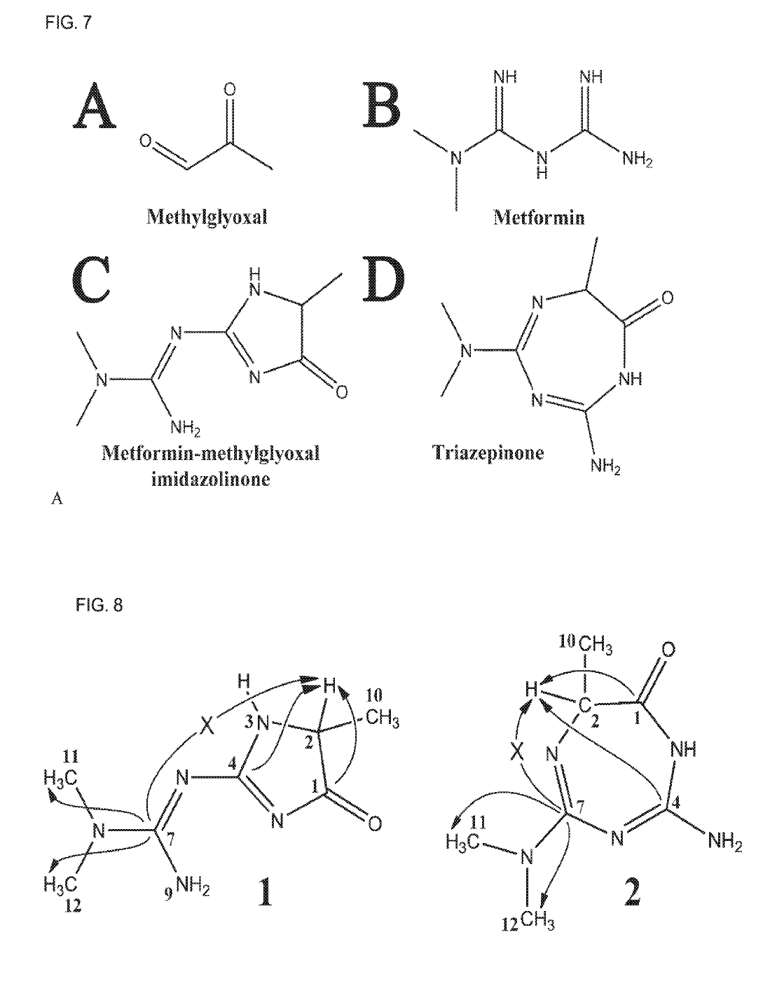

This example explores the metformin-MG reaction, determining unequivocally the structure of the product formed and developing an assay for high-throughput analysis of samples from metformin-treated T2D patients. Experiments determined that the primary product formed from the reaction between metformin and MG is not the theorized triazepinone-based structure (FIG. 7D), but in fact a five-membered imidazolinone ring structure as confirmed by X-ray diffraction (XRD) of the crystallized synthetic product (FIG. 7C). The developed LC-MS/MS multiple-reaction monitoring (MRM) based method for detection of the imidazolinone was able to determine its presence in urine samples of T2D patients currently treated in compliance with the drug metformin. The imidazolinone compound merits further study regarding the concentration of this product in human patients, pharmacokinetic and pharmacodynamic properties, and pharmacological activity, in particular at the imidazoline receptor.

### Example 2 Experimental Section

Metformin hydrochloride [1,1-dimethylbiguanide hydrochloride] was obtained from Santa Cruz Biotechnology (Santa Cruz, Calif.). 40% methylglyoxal was purchased from Sigma Aldrich.

Synthesis of metformin-methylglyoxal Product:

Metformin-MG synthesis was slightly modified from Ruggiero-Lopez et al¹². Metformin hydrochloride salt was added to 5 mL of Milli-Q water at 4° C. to a final concentration of 200 mM. Sodium hydroxide was added to the solution to a final concentration of 200 mM. Subsequently, 1.7 mL of 40% MG solution (Sigma Aldrich) was added to the metformin solution and stirred at 4° C. for one hour followed by four hours at 20° C. The resulting precipitate was filtered through Whatman 1 paper, dried, and stored under desiccation. Fresh solutions prepared in MeOH were used for subsequent analysis.

Characterization of Metformin:

The melting point was determined on a TA Q1000 differential scanning calorimeter. Tandem mass spectrometry was performed using an Agilent 6490 triple-quadrupole instrument. The maximal UV absorbance for the compound was determined with a BioSpec-mini (Shimadzu Biotech, Kyoto, Japan).

Nuclear Magnetic Resonance of Product:

The sample was dissolved in 0.7 mL of DMSO-d₆ and data were acquired on a Bruker DRX-600 spectrometer at 25° C. using a Nalorac 5 mm inverse HCN probe. ¹H chemical shifts are referenced to residual solvent (DMSO-d₅) at 2.49 ppm, and ¹³C chemical shifts are referenced to solvent (DMSO-d₆) at 39.5 ppm. The 2D gradient heteronuclear multiple-bond correlation spectroscopy (HMBC)¹⁷ spectrum was acquired in magnitude mode with 2048 real data points in t₂ and 540 points in t₁, using 8 scans per FID.

Structure and Chemical Shift Calculations:

Using Gaussian03, geometry optimization in the gas phase was performed with B3LYP/6-31G(d), followed by GIAO NMR parameter calculation using B3LYP/6-31+G(d,p) with implicit solvent. The chemical shifts of ¹³C nuclei were calculated from computed isotropic shielding using a linear model with parameters of slope −0.96 and intercept 190.0155¹⁸.

Crystallization and XRD:

The synthesized product (16 μg) was dissolved at room temp in 2:1 dimethylformamide:acetonitrile. The solvent was allowed to slowly evaporate over a period of weeks and a clear, crystalline precipitate was formed.

TABLE 3
Crystal data and structure refinement
for metformin-MG imidazolinone

| Empirical formula | C₇H₁₃N₅O |
|---|---|
| Formula weight | 183.22 |
| Temperature/K | 150.0 |
| Crystal system | monoclinic |
| Space group | P2₁/n |
| a/Å | 5.7866(3) |
| b/Å | 18.5273(11) |
| c/Å | 8.2892(5) |
| α/° | 90 |
| β/° | 93.8470(17) |
| γ/° | 90 |
| Volume/Å³ | 886.68(9) |
| Z | 4 |
| ρ_{calc}mg/mm³ | 1.373 |
| m/mm⁻¹ | 0.099 |
| F(000) | 392.0 |
| Crystal size/mm³ | 0.4 × 0.1 × 0.1 |
| Radiation | MoKα (λ = 0.71073) |
| 2Θ range for data collection | 4.396 to 52.734° |
| Index ranges | −7 ≤ h ≤ 7, −23 ≤ k ≤ 23, |
| | −10 ≤ l ≤ 10 |
| Reflections collected | 12109 |
| Independent reflections | 1814 [Rᵢₙₜ = 0.0353, R_{sigma} = 0.0237] |
| Data/restraints/parameters | 1814/0/133 |
| Goodness-of-fit on F² | 1.066 |
| Final R indexes [I >= 2σ (I)] | R₁ = 0.0471, wR₂ = 0.1209 |
| Final R indexes [all data] | R₁ = 0.0567, wR₂ = 0.1271 |
| Largest diff. peak/hole/e Å⁻³ | 0.30/−0.31 |

The clear colorless crystals were characterized by XRD analysis. Single crystals of C₇H₁₃N₅O were analyzed. A suitable crystal was selected and analyzed on a Bruker APEX-II CCD diffractometer. The crystals were elongated plates and diffracted well, but diffraction was streaky. The molecule crystallizes in the centrosymmetric, monoclinic space group P21/n with one molecule in the asymmetric unit, four in the unit cell. The structure refined well (final R1=0.0471). The crystal was kept at 150.0 K during data collection. Using Olex2¹⁹, the structure was solved with the XT²⁰ structure solution program using Direct Methods and refined with the ShelXL²⁰ refinement package using Least Squares minimization. Crystal data and structure refinement can be found in Table 3.

Quantitative Analysis of Imidazolinone Compound in Human Urine by Multiple Reaction Monitoring:

An Agilent (Palo Alto, Calif.) 1260 liquid chromatograph was utilized for achieving gradient separation using an Agilent ZORBAX Hilic Plus (2.1×50 mm) column with a 1.8 μm packing size held at 30° C. throughout the separation. A 5 μL injection of urine diluted 1:200 in Milli-Q water and snap filtered (SINGLE StEP™ eXtreme® Filter Vial 0.2 um PVDF; Thomson, Oceanside, Calif.) was used for all samples. A binary gradient consisting of 15 mM ammonium acetate in water (A) and acetonitrile (B) at a flow rate of 300 μL/min was used. The initial gradient consisted of 95% B which was held for 1 minute. After this, a linear gradient was maintained to achieve 35% B at 9 min which was held for another minute. At 10 min, another linear change was applied to get to the initial condition of 95% B at 11 min. A post-time of three min was used to re-equilibriate the column before the next analysis. The total analysis time was 14 min per sample.

Mass spectrometry was performed using an Agilent 6490 triple-quadrupole mass spectrometer with an electrospray ionization (ESI) source. Two kinds of optimization were performed for the mass spectrometer: (i) Compound specific optimization (ii) Source-dependent optimization.

TABLE 4
Optimized parameters for multiple reaction monitoring of metformin,
metformin-MG imidazolinone, and metformin-d₆.

| | Cell | | | | | |
|---|---|---|---|---|---|---|
| | Accelerator | Retention | | | | Collision |
| | Voltage | Time | | Precursor | Product | Energy |
| Compound | (V) | (min) | Polarity | Ion Mass | Ion Mass | (V) |
| Metformin | 7 | 7.55 | Positive | 130.2 | 113.2 | 13 |
| | | | | | 85.0 | 10 |
| | | | | | 71.2 | 20 |
| | | | | | 60.0 | 13 |
| | | | | | 43.1 | 65 |
| Metformin-MG | 7 | 2.45 | Positive | 184 | 113.0 | 16 |
| Imidazolinone | | | | | 71.2 | 28 |
| | | | | | 68.2 | 44 |
| | | | | | 44.1 | 36 |
| Metformin-d6 | 7 | 7.60 | Positive | 136.2 | 77.2 | 24 |
| | | | | | 59.9 | 8 |

Compound parameters were performed by first preparing the standards (metformin and synthesized metformin-MG compound) at 500 μg/mL in methanol and diluting to approximately 1 μg/mL in water. This standard was directly infused into the mass spectrometer in MS scan mode to identify the precursor mass in both positive and negative ionization modes. After this was determined, the mass spectrometer was operated in product ion scan mode and the most abundant product ions were selected. Five product ions for metformin were identified and used in the method (Table 4) while four product ions were monitored for the metformin-MG reaction product. This is higher than usual, but added to the specificity of identification of both compounds which was the goal. Once the product ions were determined, the collision energy (CE) and cell accelerator voltage (CAV) were optimized for each transition. The analyte transitions, optimized compound parameters and retention times are shown in Table 4. After this, the mass spectrometer parameters were optimized by using the Agilent Source Optimizer software to obtain optimal conditions. Details of the mass spectrometer operating conditions are presented in Table 5.

### Example 2 Results

Synthesis and Characterization of Metformin MG Product:

The synthesized product matched the description from Ruggiero-Lopez et al¹². Differential scanning calorimetry (DSC) revealed an initial melting point of 246.50° C. and a maximal melting point of 248.65° C. Maximal UV absorbance was observed at 256.5 nm. MS/MS showed fragmentation from a parent ion of 184.0 to products of 166.7, 138.9, 113.1, 85.9, 70.9, 56.0, and 44.0.

TABLE 5
HMBC NMR of the metformin-MG reaction product. Chemical shifts, multiplicity, J
couplings and HMBC correlations for the metformin-methylglyoxal reaction product.
Positions correspond to the structure diagram in FIG. 8.

| position | ¹H ppm | mult | J | integral | ¹³C ppm | HMBC |
|---|---|---|---|---|---|---|
| 1 | — | — | — | — | 190.23 | — |
| 2 | 3.600 | q | 7.0 | 1H | 53.19 | c1, 4, 10 |
| 3 | 7.830 | br. s | | 1H | — | |
| 4 | — | — | — | — | 173.92 | — |
| 7 | — | — | — | — | 159.73 | — |
| 9a | 7.35 | very br. s | | 1H | — | |
| 9b | 9.40 | very br. s | | 1H | — | |
| 10 | 1.121 | d | 7.0 | 3H | 17.63 | c1, 2 |
| 11/12 | 2.964 | s | | 6H | 36.86 | c7, 12/11 |

Abbreviations:
mult, multiplet;
s, singlet;
d, doublet;
q, quartet;
br., broad.

NMR Analysis and Interpretation:

The ¹H spectrum in DMSO-d₆ shows a CH—CH₃ spin system with chemical shifts consistent with an alanine fragment. A broadened N—CH₃ singlet with integral 6H corresponds to an N,N-dimethyl fragment with a barrier to rotation. A broad singlet at 7.83 ppm is typical of an amide H_{N} proton, and two very broad singlets at 7.35 and 9.40 ppm suggest an NH₂ group with hindered rotation (slow exchange broadening). The reaction of metformin and methylglyoxal was expected to give either five-membered ring product (1) or seven-membered ring product (2) (FIG. 8).

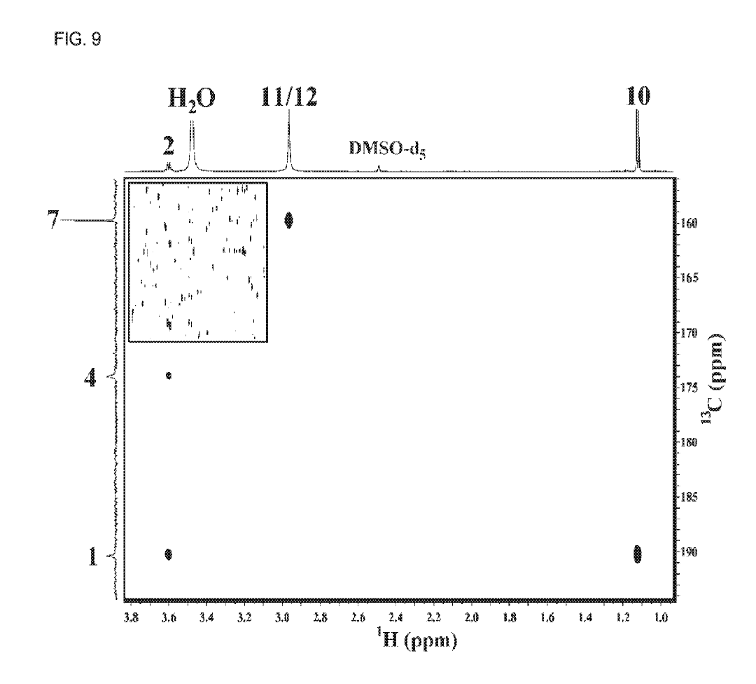

The 2D-HMBC spectrum of the reaction product in DMSO-d₆ (FIG. 9) shows correlation of the N—CH₃ proton singlet (H11/H12) with the quaternary ¹³C peak at 159.73 ppm, corresponding to C7 in structure (1). The corresponding position in structure (2) is given the same number, C7. No HMBC correlation is observed between the quartet at 3.600 ppm in the ¹H spectrum (H2) and the C7 carbon shift (arrow marked with “X” in FIG. 8). Such a correlation is expected for structure 2 (3 bonds), but not for structure 1 (5 bonds). The other two quaternary carbon peaks (C1 and C4) are correlated to H2, a distance of 3 bonds (C4) and 2 bonds (C1) in structure (1), but a distance of 4 bonds (C4) and 2 bonds (C1) in structure (2). HMBC correlations are commonly observed only for 2 and 3 bond distances, so structure (2) is ruled out. All NMR data and assignments for structure (1) are shown in Table 5.

The quaternary carbon peak at 190.23 ppm is assigned to C1, the carbonyl carbon connected to the CH—CH₃ fragment, based on HMBC correlation to the H10 (methyl doublet) peak in the ¹H spectrum. This chemical shift is more typical of a ketone carbonyl (190-220 ppm) than an amide carbonyl (170-180 ppm). Quantum-chemical calculations of ¹³C chemical shifts were performed on structures (1) and (2) using Guassian03, resulting in a prediction of 190.60 ppm for C1 in structure (1) and 173.42 ppm for C1 in structure (2) (Table 6). The reason for this unusual ¹³C chemical shift for an amide carbonyl is very likely conjugation with the extended π system (N—C4-N—C7) in structure (1), which is not present in structure (2). The predictions closely match the observed chemical shifts at all positions for structure (1), but show significant differences for structure (2), especially at C1 and C4.

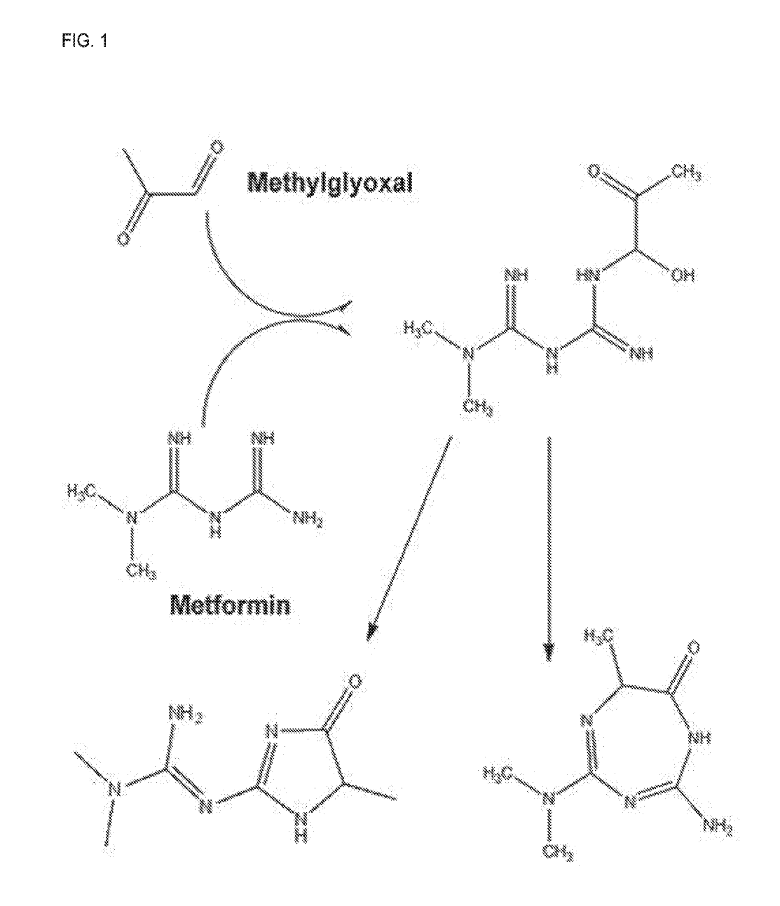

TABLE 6
Differences in chemical shifts expected for metformin-MG
possible products. Observed ¹³C chemical shifts compared
to calculated values for structures 1 and 2 of FIG. 1

| Position | ¹³C observed (ppm) | Calc. (structure 1) | Calc. (structure 2) |
|---|---|---|---|
| 1 | 190.23 | 190.60 | 173.42 |
| 2 | 53.19 | 57.33 | 62.19 |
| 4 | 173.92 | 173.75 | 145.36 |
| 7 | 159.73 | 159.02 | 155.72 |
| 10 | 17.63 | 17.27 | 13.94 |
| 11, 12 | 36.86 | 33.60 | 37.00 |

Structural Determination by XRD:

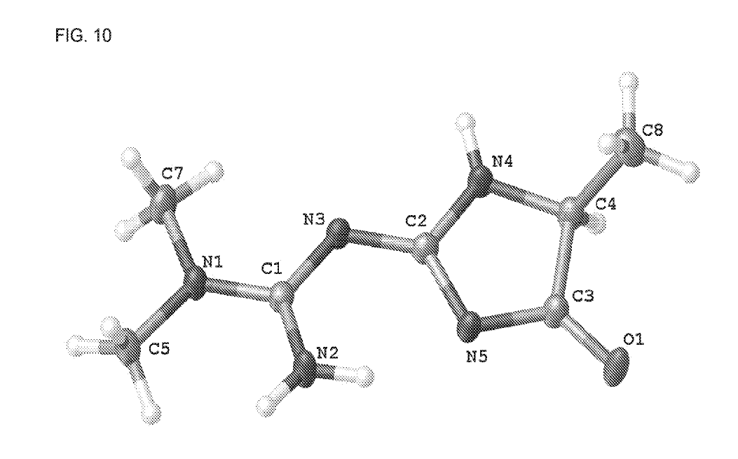

Analysis of the clear colorless crystal determined the structure as being a five-membered imidazole compound of the empirical formula C₇H₁₃N₅O with a molecular weight of 183.22 (FIG. 8C). Placement of double bonds by the software Mercury ultimately led to the conclusive determination of the structure as (E)-1,1-dimethyl-2-(5-methyl-4-oxo-4,5-dihydro-1H-imidazol-2-yl)guanidine (FIG. 10).

An extensive network of hydrogen bonding was observed connecting the molecules. As a result, the double bonds observed between C1/N3 and C2/N5 are slightly longer than would have been expected, and the single bonds between C1/N2 and C2/N3 are shorter than expected. These data imply some delocalization is probably in the molecule, which may account for some of the difficulty in peak assignment of the initial ¹H and ¹³C NMR spectra.

LC-MS/MS MRM Analysis:

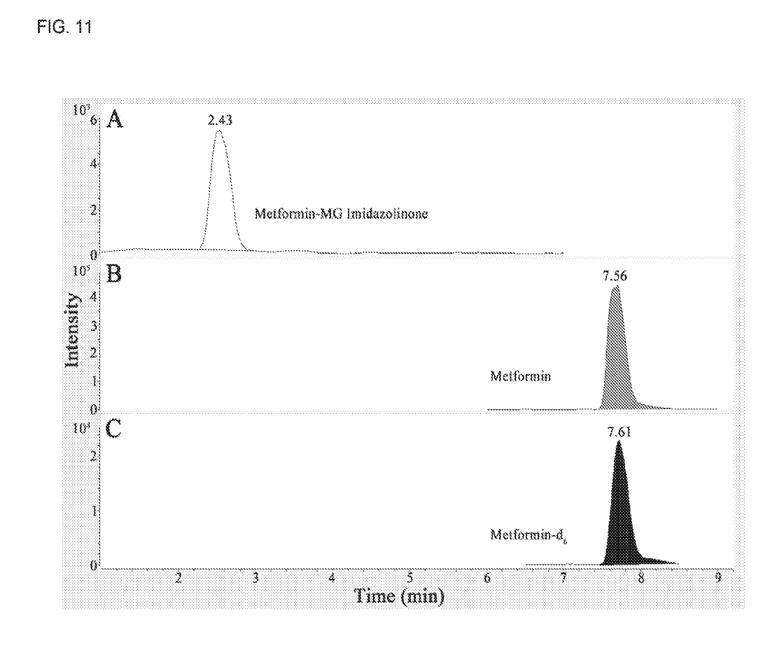

The method detection limits for metformin based on lowest standard is 0.38 ng/mL while linear range was determined between 1.5 ng/mL to 1600 ng/mL, determined by linear regression with an r² fit of 0.9986. A smaller calibration curve for imidazolinone of 1-80 ng/mL was used as concentrations were not found to be higher in 1:50 dilutions of urine samples. A detection limit of 0.07 ng/mL for imidazolinone based on a signal to noise (S/N) of greater than three was determined. Isotope-dilution method was used for quantification of metformin using metformin-d₆. Matrix spike recovery with the synthetic compound was used to verify quantification of the imidazolinone. This spike method indicated no loss of detection of the compound due to matrix or instrument. A representative parent to daughter transition for each of the three compounds monitored is shown in FIG. 11. All of the MRM transitions developed and monitored for are provided in Table 4.

### Example 2 Discussion

Pharmacological inhibition of methylglyoxal and other reactive dicarbonyls remains elusive, but still presents an admirable therapeutic target for reducing diabetic complications. While drugs like aminoguanidine have been developed as a scavenger for dicarbonyls, no drug for reducing reactive dicarbonyls has been developed solely for that purpose. However, it appears that the widely used first-line anti-hyperglycemic drug metformin scavenges methylglyoxal, and presumably other similar dicarbonyl structures, thus reducing overall dicarbonyl toxicant load. Identification of a metformin-MG scavenged product reveals that metformin has a secondary mechanism in reducing AGEs and MG burden. While it is likely that the primary route of AGE reduction observed in the past due to metformin treatment is from improved glycemic control, the unique structure of metformin also allows for this direct scavenging mechanism to directly eliminate MG from the body. Though the rate of this scavenging reaction may be slow¹³, the constant presence of metformin at concentrations as high as 15 uM in patients in compliance and at levels above plasma [MG] could indicate that it still plays a role in reducing MG levels in humans.

Previous work exploring the role of metformin in reducing methylglyoxal concentrations identified the primary product as a triazepinone-based structure¹² (FIG. 8D). While this structure does likely exist and is consistent with much of the data obtained, it is not the predominant reaction product. Stability of the seven-membered ring compound and discord with other similar types of reactions raised concern. In particular, one of the primary products resulting from the reaction of methylglyoxal and arginine is MG-H1, a hydroimidazolone five-membered ring structure³. The similarity in structure between metformin and arginine led us to infer that it was more likely that a five-membered ring was forming as a result of this reaction. Analysis of the previous data that was interpreted as a triazepinone-based structure did not conclusively disprove a five-membered ring and further studies were conducted to definitively elucidate the structure.

The analysis put forth unequivocally identifies the primary metformin-MG product as a five-membered imidazolinone with the chemical name (E)-1,1-dimethyl-2-(5-methyl-4-oxo-4,5-dihydro-1H-imidazol-2-yl)guanidine. While the MS/MS data, ¹H and ¹³C NMR data (data not shown) are consistent with that of the previous study, 2D-HMBC NMR data provided the first insight that the structure may not in fact be a seven-membered ring. Analysis of the HMBC spectrum showed the absence of an expected correlation based on the triazepinone structure outlined (FIG. 9). Crystallization followed by XRD revealed the structure of the primary product of the metformin-MG reaction as the imidazolinone compound identified in this work.

This work outlines for the first time a comprehensive method for detecting the metformin-MG scavenged product in urine from metformin-treated patients. Previous work has identified the presence of “triazepinone” in human plasma and urine^{10,14}, but a method for these results was not described in detail. Additionally, the work presented indicates that the primary product being identified, both in the previous Beisswenger studies^{10,14}, as well as in the analysis, is that of the five-membered imidazolinone. Work is ongoing to identify the urinary imidazolinone product in metformin-treated T2D patients. However, retention times in the developed MRM method are consistent between the synthesized imidazolinone product and that of the in vivo compound of the same m/z and daughter fragments.

The definitive identification of the 183 Da metformin-MG product as a five-membered ring imidazolinone rather than a triazepinone structure may appear as a slight distinction on the surface, but in fact may have larger ramifications. Recent work discovered that ligand activation of the imidazoline receptor (I1R) enhances insulin action in PC12 cells²¹. The novel I1R agonist S43126 as well as known anti-hypertensive and I1R agonist moxonidine are capable of inducing this phenomenon, with moxonidine capable of normalizing plasma insulin levels and improving glucose uptake in peripheral cells in animal models²². The metformin-MG imidazolinone identified herein bears further study as to whether it has any ligand activity at I1R and whether insulin sensitivity may be enhanced as a result.

### Example 2 References

1. Kalapos, M. P. Where does plasma methylglyoxal originate from? Diabetes Res. Clin. Pract. 2013, 99, 260-271.
2. Lo, T. W.; Westwood, M. E.; McLellan, A. C.; Selwood, T.; Thornalley, P. J. Binding and modification of proteins by methylglyoxal under physiological conditions. A kinetic and mechanistic study with N alpha-acetylarginine, N alpha-acetylcysteine, and N alpha-acetyllysine, and bovine serum albumin. J. Biol. Chem. 1994, 269, 32299-32305.
3. Ahmed, N.; Thornalley, P. J.; Dawczynski, J.; Franke, S.; Strobel, J.; Stein, G.; Haik, G. M. Methylglyoxal-derived hydroimidazolone advanced glycation end-products of human lens proteins. Invest. Ophthalmol. Vis. Sci. 2003, 44, 5287-5292.
4. Kimzey, M. J.; Yassine, H. N.; Riepel, B. M.; Tsaprailis, G.; Monks, T. J.; Lau, S. S. New site(s) of methylglyoxal-modified human serum albumin, identified by multiple reaction monitoring, alter warfarin binding and prostaglandin metabolism. Chem. Biol. Interact. 2011, 192, 122-128.
5. Thornalley, P. J.; Battah, S.; Ahmed, N.; Karachalias, N.; Agalou, S.; Babaei-Jadidi, R.; Dawnay, A. Quantitative screening of advanced glycation endproducts in cellular and extracellular proteins by tandem mass spectrometry. Biochem. J. 2003, 375, 581-592.
6. Beisswenger, P. J.; Drummond, K. S.; Nelson, R. G.; Howell, S. K.; Szwergold, B. S.; Mauer, M. Susceptibility to diabetic nephropathy is related to dicarbonyl and oxidative stress. Diabetes 2005, 54, 3274-3281.
7. Brownlee, M. Biochemistry and molecular cell biology of diabetic complications. Nature 2001, 414, 813-820.
8. Kim, Y. D.; Park, K.; Lee, Y.; Park, Y.; Kim, D.; Nedumaran, B.; Jang, W. G.; Cho, W.; Ha, J.; Lee, I.; Lee, C.; Choi, H. Metformin Inhibits Hepatic Gluconeogenesis Through AMP-Activated Protein Kinase-Dependent Regulation of the Orphan Nuclear Receptor SHP. Diabetes 2008, 57, 306-314.
9. Anonymous Effect of intensive blood-glucose control with metformin on complications in overweight patients with type 2 diabetes (UKPDS 34). UK Prospective Diabetes Study (UKPDS) Group. Lancet 1998, 352, 854-865.
10. Beisswenger, P.; Ruggiero-Lopez, D. Metformin inhibition of glycation processes. Diabetes Metab. 2003, 29, 6S95-103.
11. Kiho, T.; Kato, M.; Usui, S.; Hirano, K. Effect of buformin and metformin on formation of advanced glycation end products by methylglyoxal. Clinica Chimica Acta 2005, 358, 139-145.
12. Ruggiero-Lopez, D.; Lecomte, M.; Moinet, G.; Patereau, G.; Lagarde, M.; Wiernsperger, N. Reaction of metformin with dicarbonyl compounds. Possible implication in the inhibition of advanced glycation end product formation. Biochem. Pharmacol. 1999, 58, 1765-1773.
13. Battah, S.; Ahmed, N.; Thornalley, P. J. Kinetics and mechanism of the reaction of metformin with methylglyoxal. Int. Congr. Ser. 2002, 1245, 355-356.
14. Beisswenger, P. J. Methylglyoxal in diabetes: link to treatment, glycaemic control and biomarkers of complications. Biochem. Soc. Trans. 2014, 42, 450-456.
15. Corman, B.; Duriez, M.; Poitevin, P.; Heudes, D.; Bruneval, P.; Tedgui, A.; Levy, B. I. Aminoguanidine prevents age-related arterial stiffening and cardiac hypertrophy. Proceedings of the National Academy of Sciences 1998, 95, 1301-1306.
16. Hampp, C.; Borders-Hemphill, V.; Moeny, D. G.; Wysowski, D. K. Use of Antidiabetic Drugs in the U.S., 2003-2012. Diabetes Care 2014, 37, 1367-1374.
17. Summers, M. F.; Marzilli, L. G.; Bax, A. Complete proton and carbon-13 assignments of coenzyme B12 through the use of new two-dimensional NMR experiments. J. Am. Chem. Soc. 1986, 108, 4285-4294.
18. Lodewyk, M. W.; Siebert, M. R.; Tantillo, D. J. Computational prediction of 1H and 13C chemical shifts: a useful tool for natural product, mechanistic, and synthetic organic chemistry. Chem. Rev. 2012, 112, 1839-1862.
19. Dolomanov, O. V.; Bourhis, L. J.; Gildea, R. J.; Howard, J. A. K.; Puschmann, H. OLEX2: a complete structure solution, refinement and analysis program. Journal of Applied Crystallography 2009, 42, 339-341.
20. Sheldrick, G. M. A short history of SHELX. Acta Crystallographica Section A 2008, 64, 112-122.
21. Tesfai, J.; Crane, L.; Baziard-Mouysset, G.; Kennedy, W.; Edwards, L. P. Novel I1-imidazoline S43126 enhance insulin action in PC12 cells. Pharmacol. Rep. 2011, 63, 1442-1449.
22. Edwards, L. P.; Brown-Bryan, T. A.; McLean, L.; Ernsberger, P. Pharmacological Properties of the Central Antihypertensive Agent, Moxonidine. Cardiovascular Therapeutics 2012, 30, 199-208.
23. Kender, Z.; Fleming, T.; Kopf, S.; Torzsa, P.; Grolmusz, V.; Herzig, S.; Schleicher, E.; Racz, K.; Reismann, P.; Nawroth, P. P. Effect of metformin on methylglyoxal metabolism in patients with type 2 diabetes. Exp. Clin. Endocrinol. Diabetes 2014, 122, 316-319.

### Example 3

This example demonstrates that site-specific arginine dicarbonyl modifications of serum albumin are reduced in metformin-controlled T2DM patients.

### Example 3 Materials and Methods

Materials. All materials were HPLC grade and were purchased from Sigma-Aldrich (St. Louis, Mo.) unless otherwise noted. Synthetic peptides were custom ordered from New England Peptide (Gardner, Mass.). Sequencing-grade trypsin was purchased from Promega (Fitchburg, Wis.). Lipidex-1000 (Catalog #6008301) was purchased from PerkinElmer (Waltham, Mass.).

Subject Selection.

All subjects provided informed consent. Subjects were recruited from University Medical Center, Southern Arizona VA Health System, UPH-Kino, and El Rio diabetes and primary care clinics. Blood and urine samples were collected from 116 subjects who were instructed to fast overnight. Subjects were assigned to 3 cohorts (non-diabetic (ND) controls, type 2 diabetic subjects, and type 2 diabetic subjects with diabetic nephropathy). There are 46 subjects with normal or impaired glucose tolerance (pre-T2D), and 70 subjects with T2D. Non-diabetes is defined as fasting glucose below 100 mg/dL; pre-type 2 diabetes is fasting glucose between 100 and 125 mg/dL; diabetes is fasting glucose above 125 mg/dL. New diabetes diagnosis was based on 2-hour glucose greater than 200 mg/dl. Of the diabetic subjects, 23 had nephropathy based on a spot urine microalbumin >30 mg/g of creatinine. Forty-one diabetic subjects did not have diabetic nephropathy (DN), and six were not classified due to missing microalbumin levels. Two non-diabetic subjects had high microalbumin levels (above 30 mg/g creatinine) and were excluded from analyses involving DN. Twenty subjects (all T2D) were diagnosed with cardiovascular disease (CVD). Subjects were tested for multiple metabolic markers that include fasting insulin, fasting lipid profile, serum creatinine, uric acid, in addition to the oral glucose tolerance test (OGTT) with a 75 gram glucose solution. Subjects were also assessed for body mass index, waist circumference, blood pressure, family history of diabetes, medication history and an activity diary.

Sample Handling and Storage.

Blood was collected into heparin coated vacutainer tubes and immediately placed on ice. Blood samples were centrifuged at 4° C. and plasma was aspirated and stored at −80° C. in 200 μL aliquots. The total time between blood collection and sample storage was less than one hour.

Tryptic Digestion of Whole Plasma.

Plasma samples (200 μL aliquots) from 116 subjects were snap-thawed by immediately placing the frozen tubes into a 37° C. water bath for 10 minutes. Plasma samples were then centrifuged at 14,000×g for 5 minutes to pellet any plasma precipitates. Plasma (5 μL) from the top layer was added to 100 μL of Lipidex-1000 slurry that was buffer exchanged into an equal volume of 100 mM ammonium bicarbonate (Ambic) pH 7.4. Samples were rotated at room temperature for 30 minutes to allow for slurry mixing and delipidation of plasma. Plasma proteins were separated from slurry by centrifuging through a 0.22 μm centrifugal filter units (#UFC30GVNB, Millipore) for 5 minutes at 12,000×g. A 100 μL aliquot of 20 mM tris(2-carboxyethyl)phosphine (TCEP) pH 7.4 was added to the filtered protein samples, and these solutions were incubated at 55° C. for 30 minutes. To further denature the protein and cool the samples, the samples were then sonicated at room temperature for 10 minutes. A 100 μL aliquot of 25 mM iodoacetamide in 100 mM Ambic pH 7.4 was added to the cooled solutions and the samples were incubated in the dark at room temperature for 30 minutes. Sequencing-grade trypsin at 0.1 μg/μL in 100 mM Ambic pH 7.4 was added to each sample (50 μL, 5 μg of total trypsin) and the samples were incubated at 37° C. for 16 hours. Formic acid (5 μL) was added to stop the reactions, and the peptide samples were C18 purified using 50 mg Hypersep C18 RP cartridges (Thermo Scientific) as follows. The cartridges were washed with 5 mL acetonitrile and then equilibrated with 3×5 mL of 1% formic acid in water. The acidified peptide solutions were slowly plunged through the C18 resin at a rate of about 1 drop per second. The cartridges were washed 2×5 mL of 1% formic acid and eluted with 1 mL of 80% acetonitrile containing 1% formic acid. The peptide solutions were frozen at −80° C., lyophilized to dryness, and stored at −80° C. until LC-MRM.

LC-MRM.

A solution of 5 μg/mL synthetic stable isotope-labeled peptide AWAVAR(+10) peptide was freshly prepared in 1% formic acid and 2% acetonitrile just before MS analysis. This peptide solution was used as a diluent to reconstitute the lyophilized peptide samples. The peptide diluent (100 μL) was added to each lyophilized sample, which were then vortexed for 30 seconds. Samples were centrifuged at 15,000×g for 5 minutes, and the resuspended peptide solutions were transferred to 300 μL autosampler vial inserts.

Peptide solutions were loaded onto a ZORBAX 300SB-C18 capillary column (5 μm, 0.5×150 mm, Agilent) using a Famos autosampler (LC Packings) with a 10 μL injection loop. Peptides were loaded and eluted from the column using an LC Packings Ultimate II HPLC (Dionex) into a 4000 QTRAP using a flow rate of 40 μl/min of solvent A (0.01% TFA, 0.5% formic acid) with a 30 min linear gradient from 5% to 45% of solvent B (acetonitrile containing 0.01% TFA, 0.5% formic acid). The 4000 QTRAP was operated in MRM mode with the optimal transitions listed in Table 7. Dwell time was set to 40 ms, Q1 resolution was set to low, and Q3 resolution was set to unit. The source temperature was set at 200° C., source voltage was 5000 volts, GS1 and GS2 were set to 0 and 25 psi, respectively, and the declustering potential (DP) was set to 70 volts for all peptide parent ions. There were two technical replicates per sample.

Data Analysis.

Data analysis and MRM peak integration was performed with Multiquant software (AB Sciex). All dicarbonyl-modified and oxidized peptide peaks were manually inspected for correct assignment and peak integration. The default peak integration parameters for unmodified peptides are as follows. MQL was used as the default integration algorithm; the smoothing width was set to 1.0 points, and the peak splitting factor was set to 4 points. The area under the curve (AUC) was then calculated for modified and unmodified peptides.

Statistical analysis was performed using R version 3.0.1. Clinical variables and biomarkers were examined graphically for distributional anomalies. HbA1c, fasting glucose, triglycerides, and urinary albumin were log transformed to improve distributional characteristics in subsequent analyses. For peptide peaks, modified-to-unmodified peptide AUC ratios were computed for each sample run, and log transformed. Missing values were replaced with the average log AUC ratio from other samples at the same site. Principal components analysis (PCA) was performed using the centered and scaled log ratios. Hierarchical clustering (for normalization of MG and glucosones) was done using complete linkage and one minus Pearson's correlation for the distance metric. Pearson's correlation coefficient was also used for correlation analysis and significance testing. Group differences were assessed using analysis of variance (ANOVA), and post hoc tests were performed using Fisher's LSD (Least Significant Difference). Where non-constant variance was considered problematic, the Kruskal-Wallis test (nonparametric analog to ANOVA) was used. Provided the overall test was significant, Wilcoxon rank-sum tests were done for pairwise comparisons. Statistical significance was evaluated at the 0.05 significance level.

Normalization of MRM Data

Due to variation in assay (initial protein concentration, delipidation non-specific binding, reduction, alkylation, digestion, purification, lyophilization, and reconstitution) and in LC-MS/MS (autosampler, chromatography, instrument drift), signals of MRM modification should be normalized. Oxidized M peptide log ratios were computed for each transition, and were subsequently averaged over technical replicates for each of six M sites. PCA was performed to combine these six site-specific variables into a single measure representing oxidative modification to M. This composite variable, the first principal component, represents a normalized measure of the subject's M oxidation and will be referred to hereafter as “oxidative modification.”

Due to a missed tryptic cleavage, the unmodified arginine containing peptides are quite different from their corresponding dicarbonyl modified versions and would not serve as a useful normalization factor. While any single unmodified HSA peptide could reasonably be used, a representative value that incorporates signals from all of the unmodified sites may be a more informative normalization factor. PCA was performed on the log AUC values of the 13 unmodified peptides, averaged over technical replicates for 116 subjects. The first principal component of the 13 unmodified HSA peptides (PC1₁₃) is used as a normalization factor.

### Example 3 Results

### Identification of Oxidized and Dicarbonyl-Modified Sites In Vivo

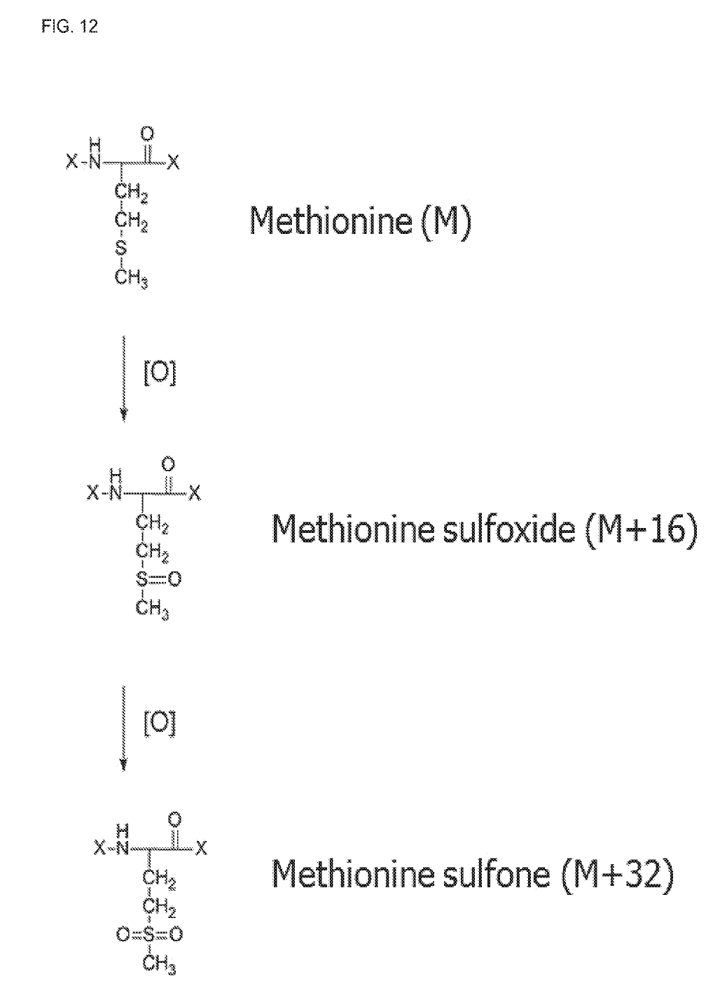

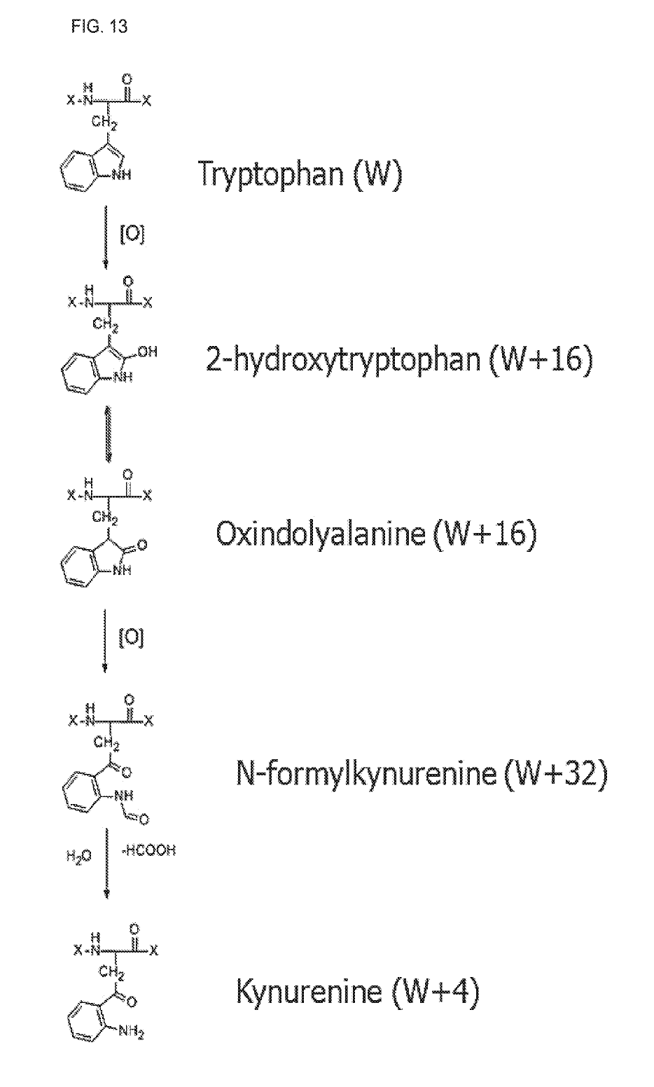

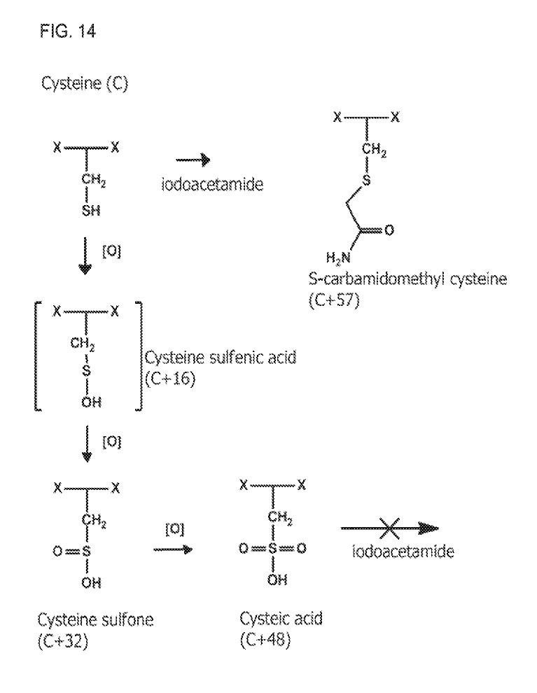

There were 13 unmodified HSA peptides monitored for each sample, six of which contain methionine (M) and were used as controls for oxidation of M (FIG. 12), one contained tryptophan (W) and was used as a control for oxidized W (FIG. 13), and one contained free cysteine (C; C34, not disulfide bonded) and was used as a control for oxidized C (FIG. 14). This leaves five peptides that were monitored that did not contain MWC oxidizable residues. For the 13 unmodified peptides, all 116 subjects had MRM signals, which exclude the presence of any HSA polymorphisms or mutations in these subjects that would result in a shift of the peptide masses at these monitored sites due to an altered amino acid sequence.

Oxidized M containing peptides gave relatively strong signals compared to corresponding unmodified peptides with peak area ratios between 0.01 and 0.1. All oxidized M peptides eluted earlier than the unmodified peptides, indicating the increased polarity/hydrophilicity of M+16 oxidation. W (W+16, W+32, W+4) and C (C+16, C+32, C+48) oxidations, however, were lower in abundance and were in most cases indistinguishable from other interfering peaks. The W and C oxidized modifications could be identified at higher sample input (the equivalent of 2 μL of plasma per LC run, as opposed to 0.5 μL of plasma per LC run), but problems with assay linearity in terms of detecting the dicarbonyl-derived hydroimidazolone modifications on arginine were observed.

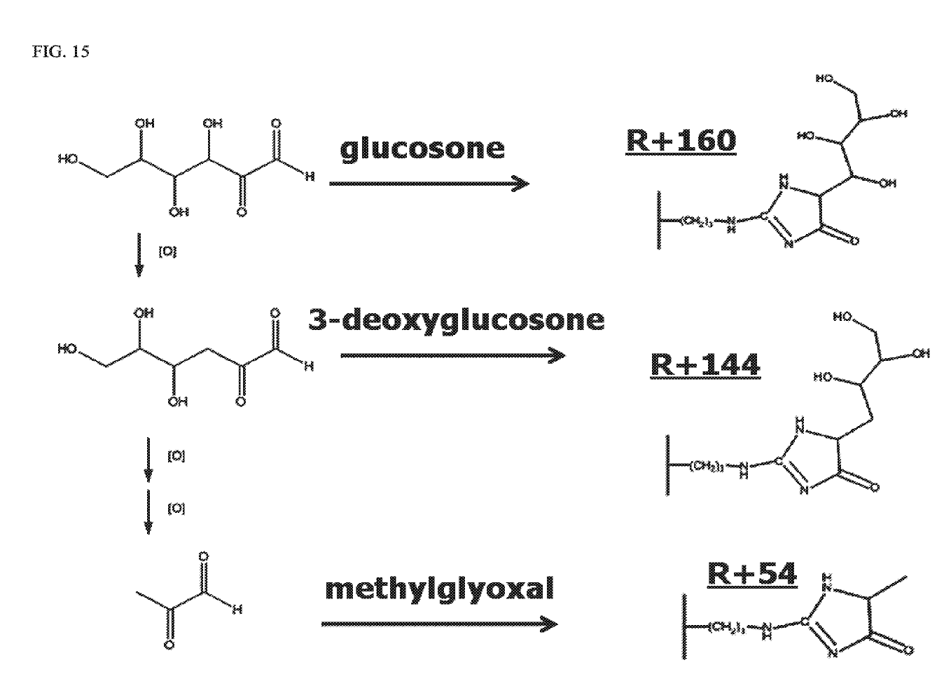

The three sites that consistently gave strong MRM signals for arginine (R) modification by dicarbonyls (FIG. 15) were R186, R257, and R428. R186 was shown to be modified by all three (MG, 3DG, glucosone) dicarbonyls, yet R257 was only shown to be modified by MG, the smaller of the dicarbonyls, and R428 was only modified by 3DG and glucosone, the larger of the dicarbonyls. Modification of R410 by dicarbonyls was not observed in any of the subjects, despite having strong MRM signals when HSA was modified in vitro in previous control studies.

### Oxidative Modification Score

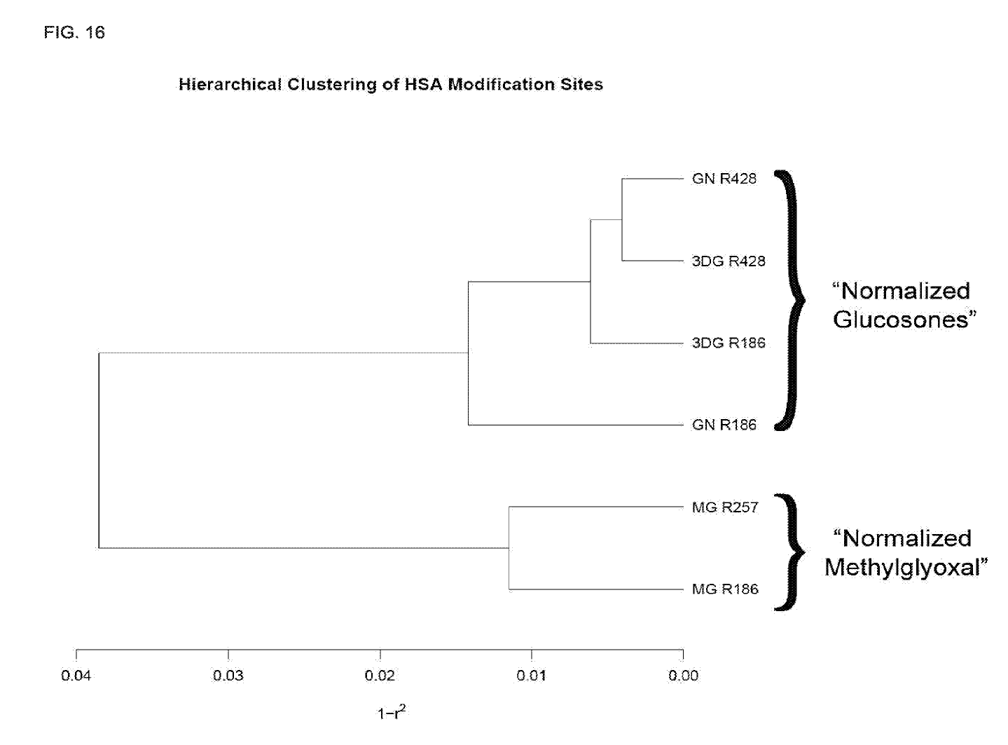

The oxidative modification PCA score captured 67% of the variation in signal at M peptide oxidation sites (data not shown). Arginine peptides modified by dicarbonyl groups were normalized by PC1₁₃. This first principal component captured 65% of variation in unmodified HSA signal (data not shown). Representing the 13 unmodified HSA peptides, PC1₁₃ was used to normalize the MRM signals for arginine containing peptides modified by dicarbonyls. Clinical analysis was performed on the log AUC ratio of dicarbonyl-modified peptides to PC1₁₃. This is equivalent to the log AUC of dicarbonyl-modified peptides minus PC1₁₃, since the principal component scores were generated with log transformed data. Thus, the principal component score for each sample (contained in PC1₁₃) was subtracted from the per-sample average of the log modified AUC for each dicarbonyl. Hierarchical clustering (FIG. 16) shows two clusters, one of MG sites and another of the larger 3DG and glucosone sites. Although these two clusters are quite similar to each other, they appear to reflect unique chemical properties of the dicarbonyls by cluster, so the clusters were treated separately in subsequent analyses. The two log ratios from MG-adducted sites R186 and R257 were averaged to give a single “MG modification” value for each subject. The four log ratios from 3DG- and glucosone-adducted sites R186 and R428 were averaged to give a single “glucosone modification” value for each subject.

### Relations Between HSA Modifications and Clinical Data

Clinical Biomarkers: Pearson correlations between HSA modifications and standard clinical measures are shown in Table 8. HSA modifications exhibit moderate positive associations with HbA1c, urine albumin, systolic BP, waist circumference (MG and Glu mods only), and age (Oxid. mods). HbA1c is more strongly correlated with most clinical variables than are HSA modifications. This suggests that the HSA modifications are measuring different information than are commonly used clinical variables.

Clinical Outcomes:

Results in Table 9 show that mean oxidative, MG, and glucosone modifications are significantly higher in T2D subjects than in non-T2D subjects (all p<0.002). As expected, HbA1c is also significantly higher in T2D than non-T2D subjects (p<0.0001). Similar trends are observed for all HSA modifications and HbA1c when comparing subjects with CVD (or DN) to those without CVD (or DN). There are no unexpected significant differences in the clinical or HSA modification data between the sexes.

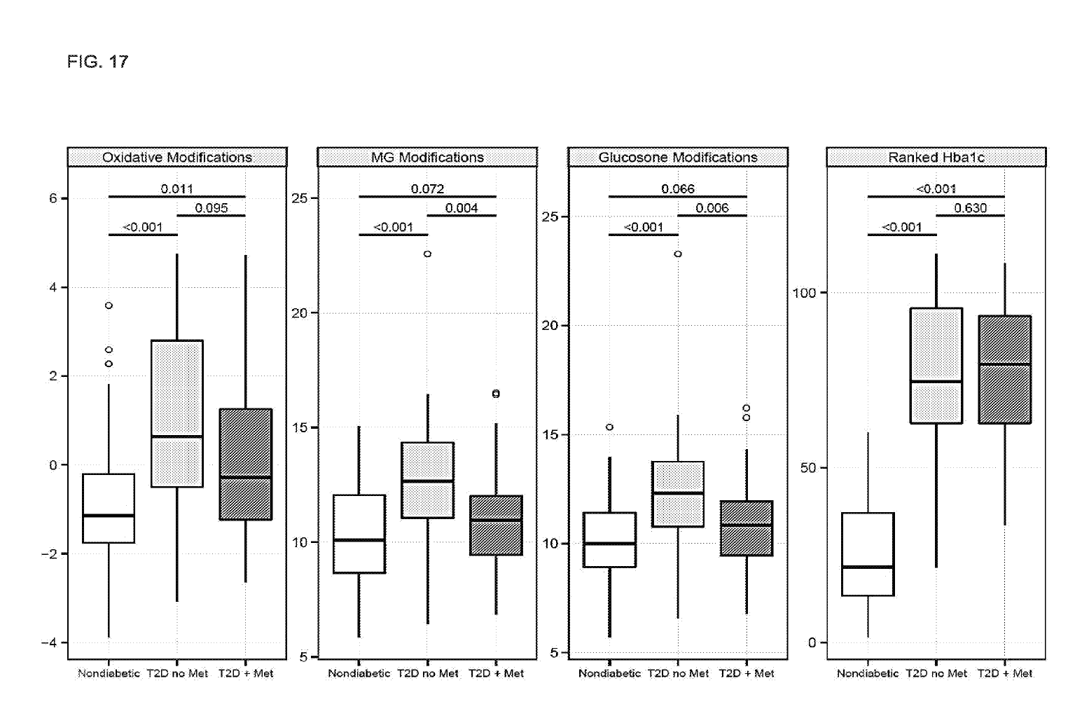

ANOVA was used to further explore the differences in three subject groups: non-diabetic (ND), T2D with metformin (T2D₊), and T2D not taking metformin (T2D₀). HSA modifications and HbA1C all show significant differences across groups (all p<0.002). While oxidative modification and HbA1c differences are associated largely by T2D/non-diabetic differences, MG and glucosone modifications also show an attenuated effect associated with metformin use (FIG. 17). There are substantial and significant decreases in both MG and glucosone modifications (p=0.0042 and p=0.0062, respectively) in T2D subjects taking metformin compared to non-metformin T2D subjects, making T2D subjects with metformin similar to ND subjects in terms of dicarbonyl modifications.

### Example 3 Discussion

### Data Reduction and Normalization.

Any single unmodified HSA peptide could have been used to adjust for assay conditions. Using PCA to identify a value that represents something akin to a weighted average of all thirteen unmodified peptides, and makes use of more information than a single unmodified peptide would provide. In order to streamline the analysis of the individual dicarbonyl modifications at the different sites, an average of two sites was chosen for MG, and an average of two sites and two dicarbonyls (glucosone, 3DG) was chosen for the glucosones based on hierarchical clustering (FIG. 16). The chemical properties (dicarbonyl size, reactivity) of MG and glucosones support the averaging of these site signals by cluster. Site specificity was not as important as the nature of the adduct for both oxidation of M and dicarbonyl adduction of R.

### Clinical Significance.

Two major aspects arise in the analysis of this data: how well these markers (oxidation, dicarbonyl adducts) capture glyco-oxidative stress, and how well glyco-oxidative stress associates with major clinical endpoints compared to traditional measures of glucose dysregulation. To answer how well these markers capture glyco-oxidative stress, it should be noted that these markers do not measure the reactive species, rather, they are the products of glyco-oxidative damage. In this sense, they are analogous to HbA1c in that they measure the history of glyco-oxidation in the plasma over a period of time. As the average half-life of HSA is 19 days, these markers should capture plasma free dicarbonyls and ROS over the lifetime of the protein. The day-to-day fluctuations of free dicarbonyls and ROS are not well studied. The degree of glyco-oxidized protein modification should account for major swings in concentrations of reactive species to average this potential variability. Due to its exploratory nature, this analysis suggests that these markers warrant further investigation, as they appear to contain different information than standard glucose measures such as HbA1c.

The use of metformin has a remarkable effect on glyco-oxidative adduct levels. It is clear in the analysis of this data that subjects taking metformin have significantly lower values for dicarbonyl modifications compared to T2D subjects who are not taking metformin. While not significant, the same trend is observed in oxidative modifications compared to T2D subjects not on metformin therapy. Metformin is associated with decreased oxidative and dicarbonyl stress, and clinical studies have demonstrated decreased levels of free dicarbonyls, dicarbonyl adducts, and oxidized adducts in diabetic subjects taking metformin relative to matched diabetic subjects not on metformin^{11,12}. The results of this study were in close agreement with a study that quantified the levels of MG-derived hydroimidazolone (MG-H1), 3DG-derived hydroimidazolone (3DG-H), and methionine sulfoxide from apoB100 in T2D subjects +/− metformin¹². T2D subjects not taking metformin had significantly higher MG, and 3DG levels than T2D subjects taking metformin. A similar trend occurred for methionine sulfoxide, though not significant. Metformin is a weak carbonyl scavenger, and its ability to decrease dicarbonyls is not thought to be primarily through direct binding of reactive carbonyls¹³. Metformin has been reported to produce a cyclic triazepinone adduct when treated with MG¹⁴, and these adducts in the urine are inversely proportional to plasma free MG levels¹⁵. Metformin does not directly scavenge free radicals¹⁶. The primary mechanisms underlying how metformin decreases dicarbonyl and oxidative stress have surprisingly not been thoroughly researched. Some promising work has looked into the upregulation of plasma and erythrocyte superoxide dismutases, catalase, and glutathione levels as a result of metformin treatment^{17, 18}. The molecular target(s) for these activities could be related to actions on the generally agreed upon target of metformin, AMP-activated protein kinase. It is widely believed, however, that the benefits of metformin are multifaceted, and not strictly attributed to the actions on a single target¹⁹. The large doses (1-3 g daily) also increase the likelihood of off-target effects. Metformin is particularly interesting because of the cardiovascular benefit in addition to the glucose-lowering effects. This could be the true utility for glyco-oxidative markers, as they are able to measure an effect of metformin that traditional measures of glucose do not. The question of whether or not these markers are able to measure cardiovascular risk is certainly worth investigating. These glyco-oxidative markers could be useful in determining if new diabetic therapies are similar to metformin in their ability to reduce protein damage by gly co-oxidation.

Additionally, these markers captured additional information about metformin treatment that HbA1c was unable to recognize. All three of the markers (MG, glucosones, M oxidation) in this study showed decreased levels among those being treated with metformin, yet HbA1c saw no change. While the implications of this finding are unknown, what is clear is that if these markers are not going to replace HbA1c, they should at the least be used in tandem to track progress of metformin treatment in reducing glyco-oxidative risk.

An ideal marker would be one that is able to predict the development of diabetic complications with greater accuracy than currently available clinical assays. This is, however, quite a lofty goal. It is more likely that markers described herein can serve as part of a panel containing multiple markers that can help to guide and optimize therapeutic regimens. Thus, early treatment of the disease would have beneficial outcome in improving the quality of life in diabetic patients. Further evaluation and final validation of these biomarkers may not only help predict patient populations that are susceptible to develop diabetes and diabetic complications, they may also serve to optimize pharmaceutical treatment during the course of therapy. These markers might also be useful in the development of new therapies that aim to reduce blood sugar as well as oxidative and carbonyl stress.

TABLE 7
Validated transitions for unmodified, glycated, and oxidized peptides.

| | | | | | | |
|---|---|---|---|---|---|---|
| parent | daughter | CE | charge | ion | peptide description | peptide sequence |

Unmodified:

| | | | | | | |
|---|---|---|---|---|---|---|
| 471.3 | 596.4 | 30 | 2 | | internal control | DDNPNLPR |
| | | | | | DDNPNLPR | |
| 481.4 | 685.4 | 30 | 2 | | R410t unmodified | FQNALLVR |
| | | | | | truncated 403-410 | |
| 575.3 | 937.5 | 25 | 2 | | internal control | LVNEVTEFAK |
| | | | | | LVNEVTEFAK | |
| | | | | | from literature | |
| 637.9 | 851.3 | 25 | 3 | y7 | R485 unmodified | RPC(57)FSALEVDETYVPK |
| 637.9 | 961.4 | 30 | 3 | b8 | R485 unmodified | RPC(57)FSALEVDETYVPK |
| 637.9 | 950.4 | 26 | 3 | y8 | R485 unmodified | RPC(57)FSALEVDETYVPK |
| 696.5 | 925.8 | 34 | 3 | y15 + 2 | R257 unmodified | VHTEC(57)C(57)HGDLLEC(57)ADDR |
| 992.2 | 780.4 | 40 | 3 | y15 + 2 | M299 unmod | SHC(57)IAEVENDEMPADLPSLAADFVESK |
| 992.2 | 1163.6 | 40 | 3 | y11 | M299 unmod | SHC(57)IAEVENDEMPADLPSLAADFVESK |
| 840.5 | 676.9 | 30 | 3 | y11 + 2 | M446 unmod | MPC(57)AEDYLSVVLNQLC(57)VLHEK |
| 671.9 | 1172.6 | 29 | 2 | y10 | M548 unmod | AVMDDFAAFVEK |
| 671.9 | 143.1 | 35 | 2 | a2 | M548 unmod | AVMDDFAAFVEK |
| 717.8 | 724.2 | 36 | 2 | y6 | M87 unmod | ETYGEMADC(57)C(57)AK |
| 717.8 | 135.8 | 50 | 2 | y1 | M87 unmod | ETYGEMADC(57)C(57)AK |
| 337.2 | 416.2 | 18 | 2 | y4 | W214 unmod | AWAVAR |
| 337.2 | 258.1 | 16 | 2 | y2 | W214 unmod | AWAVAR |
| 830.8 | 991 | 31 | 3 | y16 + 2 | C34 unmod | ALVLIAFAQYLQQC(57)PFEDHVK |
| 830.8 | 1047.5 | 31 | 3 | y17 + 2 | C34 unmod | ALVLIAFAQYLQQC(57)PFEDHVK |
| 663.4 | 809.5 | 37 | 4 | b7 | M123 unmod | LVRPEVDVMC(57)TAFHDNEETFLK |
| 663.4 | 806.4 | 24 | 4 | y13 + 2 | M123 unmod | LVRPEVDVMC(57)TAFHDNEETFLK |
| 812.4 | 1149.6 | 38 | 2 | y9 | M329 unmod | DVFLGMFLYEYAR |
| 542 | 701.4 | 26 | 3 | y5 | M329 unmod | DVFLGMFLYEYAR |
| 840.1 | 526.3 | 50 | 3 | y4 | M446 unmod | MPC(57)AEDYLSVVLNQLC |
| | | | | | | (57)VLHEK |
| 840.1 | 413.3 | 53 | 3 | y3 | M446 unmod | MPC(57)AEDYLSVVLNQLC |
| | | | | | | (57)VLHEK |

MG-modifed:

| | | | | | | |
|---|---|---|---|---|---|---|
| 469.6 | 566.3 | 21 | 3 | y9 + 2 | R410-MG modified | FQNALLVR(54)YTK |
| 469.6 | 622.2 | 24 | 3 | y4 | R410-MG modified | FQNALLVR(54)YTK |
| 469.6 | 120.1 | 55 | 3 | y1 | R410-MG modified | FQNALLVR(54)YTK |
| 660.6 | 801.5 | 31 | 4 | y20 + 3 | R257-MG modified | VHTEC(57)C(57)HGDLLEC |
| | | | | | | (57)ADDR(54)ADLAK |
| 660.6 | 421.5 | 33 | 4 | y7 + 2 | R257-MG modified | VHTEC(57)C(57)HGDLLEC |
| | | | | | | (57)ADDR(54)ADLAK |
| 660.6 | 635.6 | 32 | 4 | y21 + 4 | R257-MG modified | VHTEC(57)C(57)HGDLLEC |
| | | | | | | (57)ADDR(54)ADLAK |
| 660.6 | 237.1 | 35 | 4 | b2 | R257-MG modified | VHTEC(57)C(57)HGDLLEC |
| | | | | | | (57)ADDR(54)ADLAK |
| 660.6 | 109.6 | 90 | 4 | y2 + 2 | R257-MG modified | VHTEC(57)C(57)HGDLLEC |
| | | | | | | (57)ADDR(54)ADLAK |
| 528.7 | 605.2 | 19 | 5 | b10 + 2 | R257-MG modified | VHTEC(57)C(57)HGDLLEC |
| | | | | | | (57)ADDR(54)ADLAK |
| 528.7 | 594.7 | 19 | 5 | y10 + 2 | R257-MG modified | VHTEC(57)C(57)HGDLLEC |
| | | | | | | (57)ADDR(54)ADLAK |
| 528.7 | 659.3 | 19 | 5 | y11 + 2 | R257-MG modified | VHTEC(57)C(57)HGDLLEC |
| | | | | | | (57)ADDR(54)ADLAK |
| 528.7 | 661.7 | 19 | 5 | b11 + 2 | R257-MG modified | VHTEC(57)C(57)HGDLLEC |
| | | | | | | (57)ADDR(54)ADLAK |
| 702.8 | 627.2 | 35 | 3 | y17 + 3 | R428-MG modified | KVPQVSTPTLVEVSR(54)NLGK |
| 702.8 | 940.3 | 34 | 3 | y17 + 2 | R428-MG modified | KVPQVSTPTLVEVSR(54)NLGK |
| 702.8 | 827.3 | 37 | 3 | multiple | R428-MG modified | KVPQVSTPTLVEVSR(54)NLGK |
| 527.3 | 683.8 | 20 | 4 | y12 + 2 | R428-MG modified | KVPQVSTPTLVEVSR(54)NLGK |
| 655.6 | 244.2 | 23 | 3 | y2 | R486-MG modified | R(54)PC(57)FSALEVDETYVPK |
| 590.3 | 525.3 | 33 | 3 | y4 | R98-MG modified | QEPER(54)NEC(57)FLQHK |
| 801.7 | 1065.4 | 35 | 3 | y18 + 2 | R81-MG modified | LC(57)TVATLR(54)ETYGEMADC |
| | | | | | | (57)C(57)AK |
| 303.6 | 381.2 | 17 | 3 | y6 + 2 | R209-MG modified | FGER(54)AFK |
| 454.8 | 615.2 | 30 | 2 | b5 | R209-MG modified | FGER(54)AFK |
| 514.2 | 712.3 | 32 | 2 | y5 | R10-MG modified | SEVAHR(54)FK |
| 565.0 | 1015.3 | 34 | 2 | y8 | R186-MG modified | LDELR(54)DEGK |
| 565.0 | 796.4 | 32 | 2 | b6 | R186-MG modified | LDELR(54)DEGK |
| 376.9 | 508.2 | 18 | 3 | y8 + 2 | R186-MG modified | LDELR(54)DEGK |
| 465.3 | 816.2 | 29 | 2 | y6 | R222-MG modified | LSQR(54)FPK |
| 310.5 | 408.7 | 16 | 3 | y6 + 2 | R222-MG modified | LSQR(54)FPK |
| 560.0 | 331.2 | 50 | 3 | y3 | R348-MG modified | HPDYSVVLLLR(54)LAK |
| 528.9 | 759.3 | 33 | 2 | y6 | R472-MG modified | TPVSDR(54)VTK |
| 352.9 | 478.2 | 18 | 3 | y8 + 2 | R472-MG modified | TPVSDR(54)VTK |
| 352.9 | 429.8 | 18 | 3 | y7 + 2 | R472-MG modified | TPVSDR(54)VTK |
| 352.9 | 759.4 | 21 | 3 | y6 | R472-MG modified | TPVSDR(54)VTK |
| 606.6 | 784.4 | 30 | 2 | y6 | R218-MG modified | AWAVAR(54)LSQR |
| | | | | | (MuDPIT) | |
| 606.6 | 954.5 | 30 | 2 | y8 | R218-MG modified | AWAVAR(54)LSQR |
| | | | | | (MuDPIT) | |

3DG-modified:

| | | | | | | |
|---|---|---|---|---|---|---|
| 499.6 | 120 | 30 | 3 | b1 | R410-3DG modified | FQNALLVR(144)YTK |
| 499.6 | 611.3 | 23 | 3 | y9 + 2 | R410-3DG modified | FQNALLVR(144)YTK |
| 683 | 109.6 | 90 | 4 | y2 + 2 | R257-3DG modified | VHTEC(57)C(57)HGDLLEC |
| | | | | | | (57)ADDR(144)ADLAK |
| 910.4 | 109.6 | 100 | 3 | y2 + 2 | R257-3DG modified | VHTEC(57)C(57)HGDLLEC |
| | | | | | | (57)ADDR(144)ADLAK |
| 546.6 | 109.9 | 70 | 5 | y2 + 2 | R257-3DG modified | VHTEC(57)C(57)HGDLLEC |
| | | | | | | (57)ADDR(144)ADLAK |
| 590 | 331.2 | 55 | 3 | y3 | R348-3DG modified | HPDYSVVLLLR(144)LAK |
| 373.2 | 366.2 | 17 | 3 | y4 + 2 | R10-3DG modified | SEVAHR(144)FK |
| 831.7 | 538.2 | 50 | 3 | y4 | R81-3DG modified | LC(57)TVATLR(144)ETYGEMADC |
| | | | | | | (57)C(57)AK |
| 624 | 724.2 | 32 | 4 | y6 | R81-3DG modified | LC(57)TVATLR(144)ETYGEMADC |
| | | | | | | (57)C(57)AK |
| 465.5 | 534.6 | 20 | 4 | y11 + 3 | R98-3DG modified | QEPER(144)NEC(57)FLQHK |
| 465.5 | 456.2 | 22 | 4 | a4 | R98-3DG modified | QEPER(144)NEC(57)FLQHK |
| 406.9 | 553.3 | 19 | 3 | y8 +2 | R186-3DG modified | LDELR(144)DEGK |
| 406.9 | 495.7 | 18 | 3 | y7 + 2 | R186-3DG modified | LDELR(144)DEGK |
| 609.8 | 204.2 | 40 | 2 | y2 | R186-3DG modified | LDELR(144)DEGK |
| 333.5 | 426.2 | 18 | 3 | y6 + 2 | R209-3DG modified | FGER(144)AFK |
| 333.5 | 120 | 22 | 3 | b1 | R209-3DG modified | FGER(144)AFK |
| 651.4 | 874.5 | 30 | 2 | y6 | R218-3DG modified | AWAVAR(144)LSQR |
| 340.6 | 453.8 | 18 | 3 | y6 + 2 | R222-3DG modified | LSQR(144)FPK |
| 510.3 | 256 | 53 | 2 | R + 144 | R222-3DG modified | LSQR(144)FPK |
| 732.8 | 657 | 34 | 3 | y17 + 3 | R428-3DG modified | KVPQVSTPTLVEVSR(144)NLGK |
| 549.8 | 728.9 | 21 | 4 | y12 + 2 | R428-3DG modified | KVPQVSTPTLVEVSR(144)NLGK |
| 382.9 | 474.6 | 19 | 3 | y7 + 2 | R472-3DG modified | TPVSDR(144)VTK |
| 685.7 | 244.2 | 25 | 3 | y2 | R485-3DG modified | R(144)PC(57)FSALEVDETYVPK |

Glucosone-modified-

| | | | | | | |
|---|---|---|---|---|---|---|
| 505.1 | 120.0 | 35 | 3 | b1 | R410-Glucosone | FQNALLVR(160)YTK |
| | | | | | modified | |
| 505.1 | 619.4 | 23 | 3 | y9 + 2 | R410-Glucosone | FQNALLVR(160)YTK |
| | | | | | modified | |
| 505.1 | 276.2 | 25 | 3 | b2 | R410-Glucosone | FQNALLVR(160)YTK |
| | | | | | modified | |
| 687.1 | 109.9 | 80 | 4 | y2 + 2 | R257-Glucosone | VHTEC(57)C(57)HGDLLEC(57) |
| | | | | | modified | ADDR(160)ADLAK |
| 549.9 | 109.9 | 80 | 5 | y2 + 2 | R257-Glucosone | VHTEC(57)C(57)HGDLLEC(57) |
| | | | | | modified | ADDR(160)ADLAK |
| 549.9 | 567.9 | 23 | 5 | y9 + 2 | R257-Glucosone | VHTEC(57)C(57)HGDLLEC(57) |
| | | | | | modified | ADDR(160)ADLAK |
| 549.9 | 662.4 | 23 | 5 | y21 + 4 | R257-Glucosone | VHTEC(57)C(57)HGDLLEC(57) |
| | | | | | modified | ADDR(160)ADLAK |
| 412.3 | 561.3 | 20 | 3 | y8 + 2 | R186-Glucosone | LDELR(160)DEGK |
| | | | | | modified | |
| 617.9 | 200.1 | 50 | 2 | a2 | R186-Glucosone | LDELR(160)DEGK |
| | | | | | modified | |
| 378.6 | 459.3 | 18 | 3 | y6 + 2 | R10-Glucosone | SEVAHR(160)FK |
| | | | | | modified | |
| 567.4 | 216.8 | 40 | 2 | b2 | R10-Glucosone | SEVAHR(160)FK |
| | | | | | modified | |
| 837.1 | 538.2 | 50 | 3 | y4 | R81-Glucosone | LC(57)TVATLR(160)ETYGEMADC |
| | | | | | modified | (57)C(57)AK |
| 628.1 | 724.2 | 32 | 4 | y6 | R81-glucosone | LC(57)TVATLR(160)ETYGEMADC |
| | | | | | modified | (57)C(57)AK |
| 738.2 | 662.5 | 34 | 3 | y17 + 3 | R428-glucosone | KVPQVSTPTLVEVSR(160)NLGK |
| | | | | | modified | |
| 553.9 | 737.0 | 21 | 4 | y12 + 2 | R428-glucosone | KVPQVSTPTLVEVSR(160)NLGK |
| | | | | | modified | |

Oxidized-

| | | | | | | |
|---|---|---|---|---|---|---|
| 997.5 | 780.4 | 40 | 3 | y15 + 2 | M299-sulfoxide [O] | SHC(57)IAEVENDEM |
| | | | | | | (16)PADLPSLAADFVESK |
| 997.5 | 1163.6 | 40 | 3 | y11 | M299-sulfoxide [O] | SHC(57)IAEVENDEM |
| | | | | | | (16)PADLPSLAADFVESK |
| 820.4 | 1165.6 | 34 | 2 | y9 | M329-sulfoxide [O] | DVFLGM(16)FLYEYAR |
| 820.4 | 1101.4 | 45 | 2 | b9 | M329-sulfoxide [O] | DVFLGM(16)FLYEYAR |
| 820.4 | 713.4 | 32 | 2 | y11 + 2 | M329-sulfoxide [O] | DVFLGM(16)FLYEYAR |
| 845.4 | 526.3 | 50 | 3 | y4 | M446-ox albumin | M(16)PC(57)AEDYLSVVLNQLC |
| | | | | | | (57)VLHEK |
| 845.4 | 413.3 | 53 | 3 | y3 | M446-ox albumin | M(16)PC(57)AEDYLSVVLNQLC |
| | | | | | | (57)VLHEK |
| 845.4 | 1140.6 | 41 | 3 | y9 | M446-ox albumin | M(16)PC(57)AEDYLSVVLNQLC |
| | | | | | | (57)VLHEK |
| 667.4 | 809.5 | 37 | 4 | b7 | M123-ox albumin | LVRPEVDVM(16)C(57)TAFHDNEETFLK |
| 667.4 | 806.4 | 24 | 4 | y13 + 2 | M123-ox albumin | LVRPEVDVM(16)C(57)TAFHDNEETFLK |
| 667.4 | 879.9 | 26 | 4 | y14 + 2 | M123-ox albumin | LVRPEVDVM(16)C(57)TAFHDNEETFLK |
| 817.1 | 157.1 | 32 | 3 | a2 | C34-sulfoxide [0] | ALVLIAFAQYLQQC(16)PFEDHVK |
| | | | | | albumin | |
| 817.1 | 1133 | 26 | 3 | y19 + 2 | C34-sulfoxide [0] | ALVLIAFAQYLQQC(16)PFEDHVK |
| | | | | | albumin | |
| 817.1 | 510.4 | 29 | 3 | b5 | C34-sulfoxide [0] | ALVLIAFAQYLQQC(16)PFEDHVK |
| | | | | | albumin | |
| 822.4 | 185.2 | 40 | 3 | b2 | C34-sulfone 2[0] | ALVLIAFAQYLQQC(32)PFEDHVK |
| | | | | | albumin | |
| 822.4 | 510.4 | 28 | 3 | b5 | C34-sulfone 2[0] | ALVLIAFAQYLQQC(32)PFEDHVK |
| | | | | | albumin | |
| 822.4 | 397.3 | 33 | 3 | b4 | C34-sulfone 2[0] | ALVLIAFAQYLQQC(32)PFEDHVK |
| | | | | | albumin | |
| 827.8 | 498.3 | 45 | 3 | y4 | C34-sulfenic acid | ALVLIAFAQYLQQC(48)PFEDHVK |
| | | | | | 3[0] albumin | |
| 827.8 | 510.4 | 27 | 3 | b5 | C34-sulfenic acid | ALVLIAFAQYLQQC(48)PFEDHVK |
| | | | | | 3[0] albumin | |
| 679.9 | 594.8 | 30 | 2 | y10 + 2 | M548-sulfoxide [O] | AVM(16)DDFAAFVEK |
| 679.9 | 143.1 | 37 | 2 | a2 | M548-sulfoxide [O] | AVM(16)DDFAAFVEK |
| 725.8 | 610.7 | 34 | 2 | y10 + 2 | M87-sulfoxide [O] | ETYGEM(16)ADC(57)C(57)AK |
| 725.8 | 135.8 | 50 | 2 | Y | M87-sulfoxide [O] | ETYGEM(16)ADC(57)C(57)AK |
| 345.2 | 175.1 | 30 | 2 | y1 | W214- | AW(16)AVAR |
| | | | | | oxindolylalanine | |
| | | | | | [O] | |
| 345.2 | 416.2 | 20 | 2 | y4 | W214- | AW(16)AVAR |
| | | | | | oxindolylalanine | |
| | | | | | [O] | |
| 353.2 | 416.2 | 21 | 2 | y4 | W214-n- | AW(32)AVAR |
| | | | | | formylkynurenine | |
| | | | | | 2[O] | |
| 353.2 | 345.2 | 20 | 2 | y3 | W214-n- | AW(32)AVAR |
| | | | | | formylkynurenine | |
| | | | | | 2[O] | |
| 353.2 | 175.1 | 32 | 2 | y1 | W214-n- | AW(32)AVAR |
| | | | | | formylkynurenine | |
| | | | | | 2[O] | |
| 339.2 | 416.2 | 20 | 2 | y4 | W214-kynurenine | AW(4)AVAR |

TABLE 8
Pearson correlations between selected continuous variables.

| | Ox Mod | MG Mod | Gluc Mod | Log HbA1c |
|---|---|---|---|---|
| MG Mod | 0.428** | | | |
| Gluc Mod | 0.481** | 0.986** | | |
| Log HbA1c | 0.219* | 0.247** | 0.276** | |
| 2 Hour Glucose | 0.188 | 0.188 | 0.203* | 0.845** |
| Log Urine Albumin | 0.367** | 0.319** | 0.335** | 0.389** |
| Log TG | 0.154 | 0.125 | 0.106 | 0.489** |
| HDL | −0.091 | −0.103 | −0.093 | −0.350** |
| SBP | 0.217* | 0.212* | 0.219* | 0.342** |
| WC | 0.150 | 0.196* | 0.199* | 0.365** |
| BMI | 0.075 | 0.167 | 0.171 | 0.268* |
| Age | 0.271** | 0.149 | 0.140 | 0.188* |

Significance is marked by * for p < 0.05 or ** for p < 0.01. Oxidative modifications (Ox Mod) are moderately associated with dicarbonyl modifications. MG modifications (MG Mod) and glucosone modifications (Gluc Mod) are very strongly associated. All HSA modifications are weakly associated with log HbA1c. While log HbA1c is moderately correlated with most clinical variables, HSA modifications are moderately correlated only with log Urine Albumin. SBP, WC, and age have weaker associations with HSA modifications.

TABLE 9
Sample size and mean (standard deviation) for subject groups.

| | HbA1c | | HSA Modifications | | | |
|---|---|---|---|---|---|---|
| | N | Log HbA1c | N | Oxidative | MG | Glucosone |
| T2D | | | | | | |
| Yes | 66 | 76.93 (21.91)¹ | 70 | 0.50 (2.05) | 11.83 (2.89) | 11.61 (2.77) |
| No | 45 | 25.30 (16.95)¹ | 46 | −0.76 (1.68) | 10.20 (2.25) | 10.07 (2.08) |
| p-value | | 0.0000² | | 0.0008 | 0.0017 | 0.0018 |
| CVD | | | | | | |
| Yes | 20 | 2.15 (0.27) | 20 | 1.04 (1.85) | 12.28 (3.50) | 12.10 (3.63) |
| No | 89 | 1.90 (0.30) | 93 | −0.26 (1.94) | 10.93 (2.57) | 10.74 (2.34) |
| p-value | | 0.0010 | | 0.0068 | 0.0497 | 0.0365 |
| DN | | | | | | |
| Yes | 23 | 2.15 (0.30) | 23 | 0.56 (1.89) | 12.48 (3.26) | 12.29 (3.18) |
| No | 82 | 1.90 (0.30) | 84 | −0.35 (1.95) | 10.82 (2.55) | 10.65 (2.39) |
| p-value | | 0.0005 | | 0.0476 | 0.0108 | 0.0082 |
| T2D/Metformin | | | | | | |
| T2D + Met | 35 | 78.46 (20.87)¹ | 37 | 0.22 (1.88) | 11.04 (2.42) | 10.90 (2.26) |
| T2D no Met | 31 | 75.21 (23.25)¹ | 33 | 0.82 (2.22) | 12.71 (3.14) | 12.40 (3.10) |
| Nondiabetic | 45 | 25.30 (16.05)¹ | 46 | −0.76 (1.68) | 10.20 (2.25) | 10.07 (2.08) |
| p-value | | 0.0000² | | 0.0015 | 0.0002 | 0.0003 |

Test p-value underneath group means. For HSA modifications, subjects who take metformin are not different from those who do not take metformin. However, the group of sbjects who do not take metformin includes non-diabetics and T2D subjects. Upon splitting this group (T2D/Metformin), there are significant differences in HSA modifications.
¹mean (standard deviation) are ranked HbA1c
²p-value from Kruskal-Wallis test

### Example 3 References

1. Ahmed N and Thornalley P J. Advanced glycation endproducts: what is their relevance to diabetic complications? Diabetes Obes Metab. 2007; 9(3): 233-45.
2. Duckworth W, Abraira C, Moritz T, Reda D, Emanuele N, Reaven P D, et al. Glucose control and vascular complications in veterans with type 2 diabetes. N Engl J Med. 2009; 360(2): 129-39.
3. Gerstein H C, Miller M E, Byington R P, Goff D C, Jr., Bigger J T, Buse J B, et al. Effects of intensive glucose lowering in type 2 diabetes. N Engl J Med. 2008; 358(24): 2545-59.
4. Patel A, MacMahon S, Chalmers J, Neal B, Billot L, Woodward M, et al. Intensive blood glucose control and vascular outcomes in patients with type 2 diabetes. N Engl J Med. 2008; 358(24): 2560-72.
5. Lapolla A, Fedele D and Traldi P. Glyco-oxidation in diabetes and related diseases. Clin Chim Acta. 2005; 357(2): 236-50.
6. Ahmed N, Dobler D, Dean M and Thornalley P J. Peptide mapping identifies hotspot site of modification in human serum albumin by methylglyoxal involved in ligand binding and esterase activity. J Biol Chem. 2005; 280(7): 5724-32.
7. Stevens L A, Coresh J, Greene T and Levey A S. Assessing kidney function—measured and estimated glomerular filtration rate. N Engl J Med. 2006; 354(23): 2473-83.
8. Levey A S, Bosch J P, Lewis J B, Greene T, Rogers N, Roth D, et al. A More Accurate Method To Estimate Glomerular Filtration Rate from Serum Creatinine: A New Prediction Equation. Annals of Internal Medicine. 1999; 130(6): 461-70.
9. Prinsen B and de Sain-van der Velden M G M. Albumin turnover: experimental approach and its application in health and renal diseases. Clinica Chimica Acta. 2004; 347(1-2): 1-14.
10. Himmelfarb J and McMonagle E. Albumin is the major plasma protein target of oxidant stress in uremia. Kidney Int. 2001; 60(1): 358-63.
11. Beisswenger P J, Howell S K, Touchette A D, Lal S and Szwergold B S. Metformin reduces systemic methylglyoxal levels in type 2 diabetes. Diabetes. 1999; 48(1): 198-202.
12. Rabbani N, Chittari M V, Bodmer C W, Zehnder D, Ceriello A and Thornalley P J. Increased glycation and oxidative damage to apolipoprotein B100 of LDL cholesterol in patients with type 2 diabetes and effect of metformin. Diabetes. 2010; 59(4): 1038-45.
13. Engelen L, Lund S S, Ferreira I, Tarnow L, Parving H H, Gram J, et al. Improved glycemic control induced by both metformin and repaglinide is associated with a reduction in blood levels of 3-deoxyglucosone in nonobese patients with type 2 diabetes. Eur J Endocrinol. 2011; 164(3): 371-9.
14. Ruggiero-Lopez D, Lecomte M, Moinet G, Patereau G, Lagarde M and Wiernsperger N. Reaction of metformin with dicarbonyl compounds. Possible implication in the inhibition of advanced glycation end product formation. Biochem Pharmacol. 1999; 58(11): 1765-73.
15. Beisswenger P and Ruggiero-Lopez D. Metformin inhibition of glycation processes. Diabetes Metab. 2003; 29(4 Pt 2): 6S95-103.
16. Khouri H, Collin F, Bonnefont-Rousselot D, Legrand A, Jore D and Gardes-Albert M. Radical-induced oxidation of metformin. European Journal of Biochemistry. 2004; 271(23-24): 4745-52.
17. Pavlovic D, Kocic R, Kocic G, Jevtovic T, Radenkovic S, Mikic D, et al. Effect of four-week metformin treatment on plasma and erythrocyte antioxidative defense enzymes in newly diagnosed obese patients with type 2 diabetes. Diabetes Obes Metab. 2000; 2(4): 251-6.
18. Faure P, Rossini E, Wiernsperger N, Richard M J, Favier A and Halimi S. An insulin sensitizer improves the free radical defense system potential and insulin sensitivity in high fructose-fed rats. Diabetes. 1999; 48(2): 353-7.
19. Scarpello J H and Howlett H C. Metformin therapy and clinical uses. Diab Vasc Dis Res. 2008; 5(3): 157-67.

### Example 4

This example demonstrates site specific modification of the human plasma proteome by methylglyoxal.

### Example 4 Materials and Methods

Materials.

HPLC grade solvents were purchased from Sigma-Aldrich unless otherwise noted. Sequencing grade trypsin was purchased from Promega (Fitchburg, Wis.). Prodan (6-Propionyl-2-dimethylaminonaphthalene) was a product of Anaspec Inc (Fremont, Calif., catalog #88212, lot #64774). Fatty acid-free human serum albumin (catalog # A3782) and 40% methylglyoxal solution were obtained from Sigma-Aldrich. Lipidex-1000 was acquired from PerkinElmer (Waltham, Mass.).

Subject Selection.

All subjects provided informed consent. Subjects were recruited from the University Medical Center, University Physicians Healthcare-Kino, Southern Arizona VA Health Care System, and El Rio diabetes and primary care clinics.

Sample Handling and Storage.

Blood was collected into heparin coated vacutainer tubes and immediately placed on ice. Blood samples were centrifuged at 4° C. and plasma was aspirated and stored at −80° C. in 200 μL aliquots. The total time between blood collection and sample storage was less than one hour.

Plasma Protein Fractionation and Modification.

Plasma (50 μl) from a healthy subject was diluted to 600 μl with TBS (Tris-buffered saline) pH 7.4 and centrifuged through a 0.2 μm pore size spin filter to remove particulates. The sample was incubated with 500 μM MG at 37° C. for 24 hours. The sample was buffered exchanged into 100 mM ammonium bicarbonate pH 7.4 using Vivaspin centrifuge concentrators (MWCO 3K).

Tryptic Digestion.

Modified plasma was reduced with DTT (20 mM in 100 mM ammonium bicarbonate pH 7.4) for 30 minutes at 55° C. and alkylated with iodoacetamide (55 mM in 100 mM ammonium bicarbonate pH 7.4) for 30 minutes at room temperature in the dark. Protein was then digested with trypsin (protein to trypsin at 50:1 w/w ratio) overnight at 37° C. Peptides were desalted using Hypersep C18 columns (Thermo Scientific), lyophilized, and resuspended in 10 μL of 1% TFA immediately prior to LC-MS/MS.

LC-MS/MS for Plasma Proteins.

LC-MS/MS analysis of in-solution trypsin digested-proteins (Shevchenko et al., 1996) was carried out using a LTQ Orbitrap Velos mass spectrometer (Thermo Fisher Scientific, San Jose, Calif.) equipped with an Advion nanomate ESI source (Advion, Ithaca, N.Y.), following ZipTip (Millipore, Billerica, Mass.) C18 sample clean-up according to the manufacturer's instructions. Peptides were eluted from a C18 precolumn (100-μm id×2 cm, Thermo Fisher Scientific) onto an analytical column (75-μm ID×10 cm, C18, Thermo Fisher Scientific) using a using a 5% hold of solvent B (acetonitrile, 0.1% formic acid) for 5 min, followed by a 5-7% gradient of solvent B over 5 min, 7-15% gradient of solvent B over 45 min, 15-35% gradient of solvent B over 60 min, 35-40% gradient of solvent B over 28 min, 40-85% gradient of solvent B over 5 min, 85% hold of solvent B for 10 min and finally a return to 5% in 1 minute and another 10 minute hold of 5% solvent B. All flow rates were 400 nl/min. Solvent A consisted of water and 0.1% formic acid. Data dependent scanning was performed by the Xcalibur v 2.1.0 software (Andon et al., 2002) using a survey mass scan at 60,000 resolution in the Orbitrap analyzer scanning m/z 350-1600, followed by collision-induced dissociation (CID) tandem mass spectrometry (MS/MS) of the fourteen most intense ions in the linear ion trap analyzer. Sequest was set up to search human proteins downloaded from UniProtKB on Aug. 6, 2013. Variable modifications considered during the search included methionine oxidation (15.995 Da), cysteine carbamidomethylation (57.021 Da), as well as two products of the adduction of arginine residues by MG, methylglyoxal-derived hydroimidazolone (MG-H1; 54.011 Da) and methylglyoxal-derived dihydroxyimidazoldine (MG-DH; 72.021 Da). At the time of the search, the Human UniProt database contained 88,323 entries. Proteins were identified at 95% confidence with XCorr scores (Qian et al., 2005) as determined by a reversed database search using the Percolator algorithm (per-colator.com) (Spivak et al., 2009). Identified modified peptides were considered with a q-value<0.01 (Kall et al., 2008). In order to manually validate sites for adduction, experiments employed the following criteria: B and Y ion series must provide coverage at the site of modification; tryptic peptides identified with a C-terminal MG modification were not included (false positives); and unmodified peptides from the protein must also be identified.

Prodan Displacement Assay.

A solution of human serum albumin (HSA) (20 ml at 10 mg/ml) in 1×PBS pH 7.4 was prepared. A slurry (10 ml) of Lipidex-1000 in methanol was buffer exchanged into 1×PBS and the buffer decanted from the aqueous slurry. The HSA solution was added to the slurry and the mixture was rotated at room temperature for 30 min. The Lipidex slurry was completely removed from HSA by plunging through a 0.22 μm filter using a 60 ml syringe. Prodan (25 mg) was dissolved in of 40% ethanol, 60% methanol (5 ml at a final concentration is 5 mg/ml). A portion (10 ml) of delipidated HSA (10 mg/ml) was incubated with 666.7 μl of the prodan solution (1:10 molar ratio HSA:prodan), and this mixture was rotated at room temperature for 30 minutes. The HSA-prodan mixture was placed on dry ice and acetone precipitated by adding 40 ml of acetone at −20° C., which was mixed and centrifuged at 3000×g for 10 min to pellet the precipitated HSA-prodan. Two additional washes of cold acetone were used to remove any free prodan. The HSA pellet was brought to 10 ml with PBS and mixed and resuspended.

A 2-fold serial dilution of MG in 1×PBS pH 7.4 was prepared starting from a stock solution of 10 mM. An equal volume of HSA-prodan and MG solution (200 μl each) was mixed in triplicate reactions for each MG concentration and incubated at 37° C. for 30 min. An aliquot (300 μl) of each reaction was read using a Gemini XPS Fluorescence Microplate Reader (Molecular Devices) in endpoint mode with 360 nm excitation, 420 nm filter cut-off, and 465 nm emission.

Modification of Peptides with MG and Transition Optimization of Synthetic Peptides.

Tryptic peptide R257 was synthesized, HPLC purified, lyophilized, and resuspended in 18Ω Milli-Q grade water to 5 mg/ml, with 150 μl aliquots used for each reaction. Iodoacetamide (200 mM; 20 μl) in 20 mM ammonium bicarbonate pH 7.4 was added, and the solution incubated in the dark at room temperature for 1 hr. MG (150 μl of 10 mM) in 2×PBS pH 7.4 was added to R257 peptide solution, and the reaction was incubated at 37° C. for 2 hrs. The reaction was terminated by the addition of formic acid (10 μl), and the modified peptide was desalted with 50 mg Hypersep C18 RP cartridges (Thermo Scientific) and eluted with 80% acetonitrile containing 0.1% TFA. The peptides were diluted four-fold with a solution of 0.5% formic acid in water prior to infusion.

Peptide solution was infused at 3 μl/min into a 4000 QTRAP (Applied Biosystems/MDS Sciex) equipped with a Turbo Spray ion source and the manual transition optimization was performed. The source temperature was set at 200° C., source voltage was 5000 volts, GS1 and GS2 were set to 0 and 25 psi, respectively, and the declustering potential was set to 70 volts for all peptide parent ions.

Theoretical peptide fragment ion masses were obtained using the MS-Product function in ProteinProspector (Clauser et al., 1999). While the individual peptide solutions were infused into the QTRAP, mass spectra of parent ions for each charge state (+2, +3, +4) were analyzed using enhanced MS (EMS) mode in Analyst v. 1.4 (AB Sciex). Parent ions from each charge state (noted above) were fragmented in enhanced product ion (EPI) mode (MS/MS) and a list of potential MRM transitions was generated. From all of the possible intense ions generated from the MS/MS spectra, only b or y ions were selected for MRM optimization that were in agreement with the site of modification. In MRM mode, transitions were monitored as the collision energy was ramped from 5 to 100 eV and 1-5 candidate transitions were chosen per peptide.

Nine transitions were optimized for the albumin peptide R257. Ultimately the four highest transitions were utilized in human sample analysis. The optimized transitions are shown in Table 10.

Tryptic Digestion and LC-MRM of Human Samples.

Twelve human plasma samples (200 μl aliquots) were snap thawed by immediately placing the frozen tubes into a 37° C. water bath for 10 min. Plasma samples were then centrifuged at 14,000×g for 5 min to pellet precipitate. Cleared plasma (5 μl) was added to 100 μl of Lipidex-1000 slurry and buffer exchanged into an equal volume of 100 mM ammonium bicarbonate (Ambic) pH 7.4. Samples were rotated at room temperature for 30 min to allow for slurry mixing and delipidation of plasma. Plasma proteins were separated from slurry by centrifuging through 0.22 μm centrifugal filter units (Cat# UFC30GVNB, Millipore) for 5 min at 12,000×g. An aliquot (100 μl) of 20 mM tris(2-carboxyethyl)phosphine (TCEP) pH 7.4 was added to the filtered protein samples, and these solutions were incubated at 55° C. for 30 min. To further denature the protein and cool the samples, they were subsequently sonicated at room temperature for 10 min. Aliquots (100 μl) of 25 mM iodoacetamide in 100 mM Ambic pH 7.4 were added to the cooled solutions and the samples incubated in the dark at room temperature for 30 min. Sequencing-grade trypsin (50 at 0.1 μg/μl in 100 mM Ambic pH 7.4) was added to each sample (5 μg of total trypsin) and the samples incubated at 37° C. for 16 hrs. Formic acid (5 μl) was added to stop the reactions, and the peptide samples were C18 purified using 50 mg Hypersep C18 RP cartridges (Thermo Scientific) as follows. The cartridges were washed with 5 ml acetonitrile and then equilibrated with 3×5 ml of 1% formic acid in water. The acidified peptide solutions were slowly passed through the C18 resin at a rate of about 1 drop per second. The cartridges were washed 2×5 ml of 1% formic acid and eluted with 1 ml of 80% acetonitrile containing 1% formic acid. The peptide solutions were frozen at −80° C., lyophilized to dryness, and stored at −80° C. until analysis by LC-MRM.

Peptide solutions were loaded onto a ZORBAX 300SB-C18 capillary column (5 μm, 0.5×150 mm, Agilent) using a Famos autosampler (LC Packings) with a 10 μl injection loop. Peptides were loaded and eluted from the column using an LC Packings Ultimate II HPLC (Dionex) into a 4000 QTRAP at a flow rate of 40 μl/min of solvent A (0.01% TFA, 0.5% formic acid) with a 30 min linear gradient from 5% to 45% of solvent B (acetonitrile containing 0.01% TFA, 0.5% formic acid). The 4000 QTRAP was operated in MRM mode with the optimal R257 transitions. Dwell time was set to 40 ms, Q1 resolution was set to low, and Q3 resolution was set to unit. The source temperature was set at 200° C., source voltage was 5000 volts, GS1 and GS2 were set to 0 and 25 psi, respectively, and the declustering potential (DP) was set to 70 volts for all peptide parent ions. There were two technical replicates per sample.

### Example 4 Results

MG Sites in the Plasma Proteome.

MG primarily reacts with arginine residues to form relatively stable ring structures-dihydroxyimidazolidine (MG-DH; R+72) and hydroimidazolone (MG-HI; R+54). This adduction results in a net loss of positive charge from the arginine site, as arginine is positively charged under physiological conditions, whereas the ring adducts are uncharged. In order for MG adducts to be detected using MS/MS, they must not be labile during ion activation. Upon in vitro treatment of plasma protein with MG, several MG hotspots were identified, revealing candidate sites on HSA (including R257), serotransferrin, haptoglobin, hemopexin, and Ig lambda-2 chain C regions (Table 11). The sites identified for HSA are consistent with previous studies performed with pure HSA protein, though fewer total sites were recognized due to the complexity of the plasma sample (Kimzey et al., 2011). The identified proteins that harbor sites for MG modification are considered abundant within the plasma proteome (Kuzyk et al., 2009). Data-dependent MS/MS sequencing in typical shotgun proteomics workflows tends to preferentially identify highly abundant peptides relative to less abundant peptides. As such, the identification of modified abundant proteins and abundant peptides is expected. These peptides contained arginine (R) with atomic mass unit increases of R+54 and R+72 that fragment along the peptide backbone with minimal neutral loss of the modified arginine moiety. The findings corroborate other studies that have determined that low-energy collision induced activation is appropriate for detection of MG hotspots (Brock et al., 2007, Gao and Wang, 2006).

When adding potential modifications to database searches, false positives can result and it is therefore necessary to manually validate the MG-modified spectra by using criteria specific for this modification. Working under the notion that trypsin would have inhibited cleavage at MG-modified arginine, any results that gave a sequence with a C-terminal modified arginine were treated as false positives. The sequences of peptides where MG modification was identified with the intrapeptide arginine (R) modification site are listed in Table 11.

Functional Consequences of MG Modification on HSA-Drug Binding.

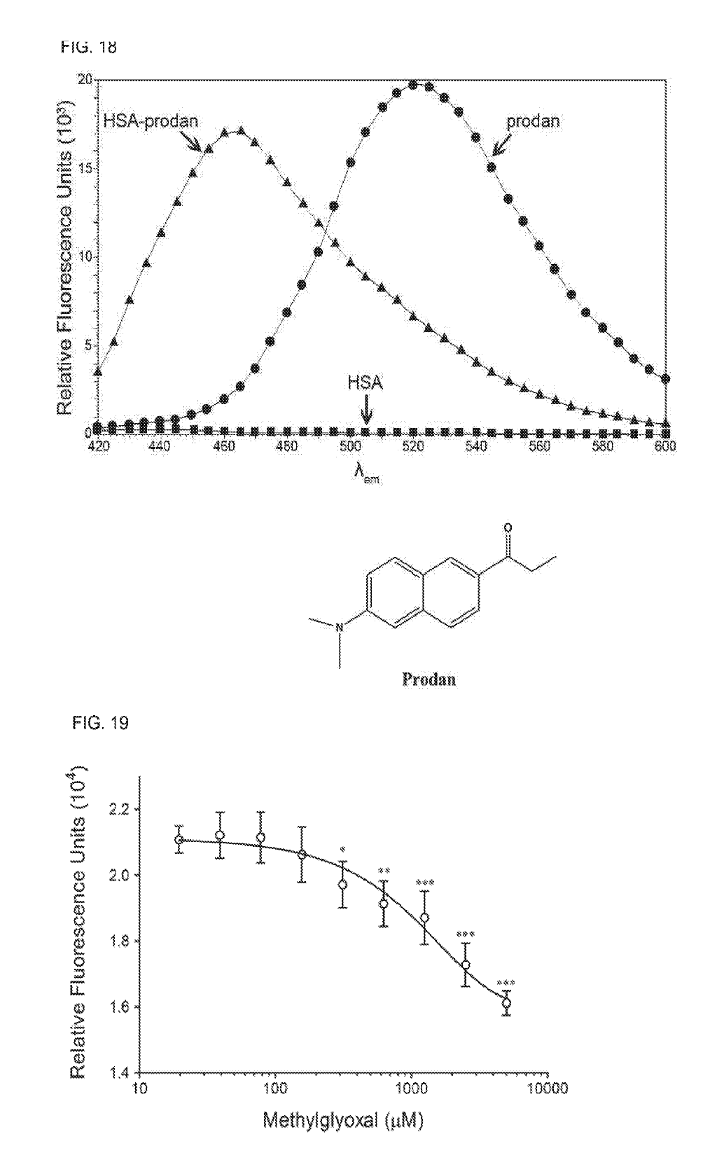

In order to visualize the functional effects of R257 modification, which resides in drug site I of HSA, prodan was utilized. The unique spectral properties of prodan make it an effective tool for the study of drug site I (FIG. 18). When prodan is free in solution it exhibits a fluorescent maximum at 520 nm when excited at 380 nm. However, when prodan is bound in drug site I of HSA, it undergoes a blue shift of 55 nm and this complex absorbs light at 380 nm yielding a fluorescent maximum at 465 nm (FIG. 18). This phenomenon may be the result of radiation-less energy transfer between W214 and bound prodan (Moreno and Gonzalez-Jimenez, 1999). The prodan-HSA complex can be monitored spectrophometrically because HSA alone does not exhibit autofluorescence in this range.

Using the fluorescent endpoint of 465 nm for the prodan-HSA complex permitted the study of the impact of MG adduction on drug site I. Any perturbations of this site can be indirectly measured by the displacement of prodan, which will ultimately decrease fluorescence at 465 nm. Therefore, maximum signal indicates unaffected prodan-HSA binding. Free prodan was purified from the HSA-prodan complex, and this complex was treated with a dilution of MG in triplicate reactions. Over time, prodan is naturally displaced from the HSA drug site I pocket, and the time point of 30 min was chosen because it permits sufficient time for MG to react with the protein complex. The “leakiness” of prodan from the complex does not permit analysis of sophisticated kinetic measurements, however the measure of the complex can be determined relative to MG treatment at any given time point.

The effect of MG on the prodan-HSA complex is illustrated in FIG. 19. Relative to the unmodified control, where no MG was added to prodan-HSA (75 μM), MG treatment displaces prodan at concentrations above 300 μM in vitro. The data indicate that drug site I is targeted by MG in terms of binding to the prodan fluorescent probe. Currently, there is no available crystal structure of HSA bound to prodan, but from this data it seems that arginine side chains are not the only residues which influence the prodan interaction with drug site I. Thus, MG-modification of arginine reduces, but does not completely abolish, prodan binding in a dose dependent manner. Nevertheless, eventual displacement of prodan from this site at higher MG concentrations is further evidence that drug site I is a target for MG adduction.

Identification of MG-Modified R257 in Human Plasma.

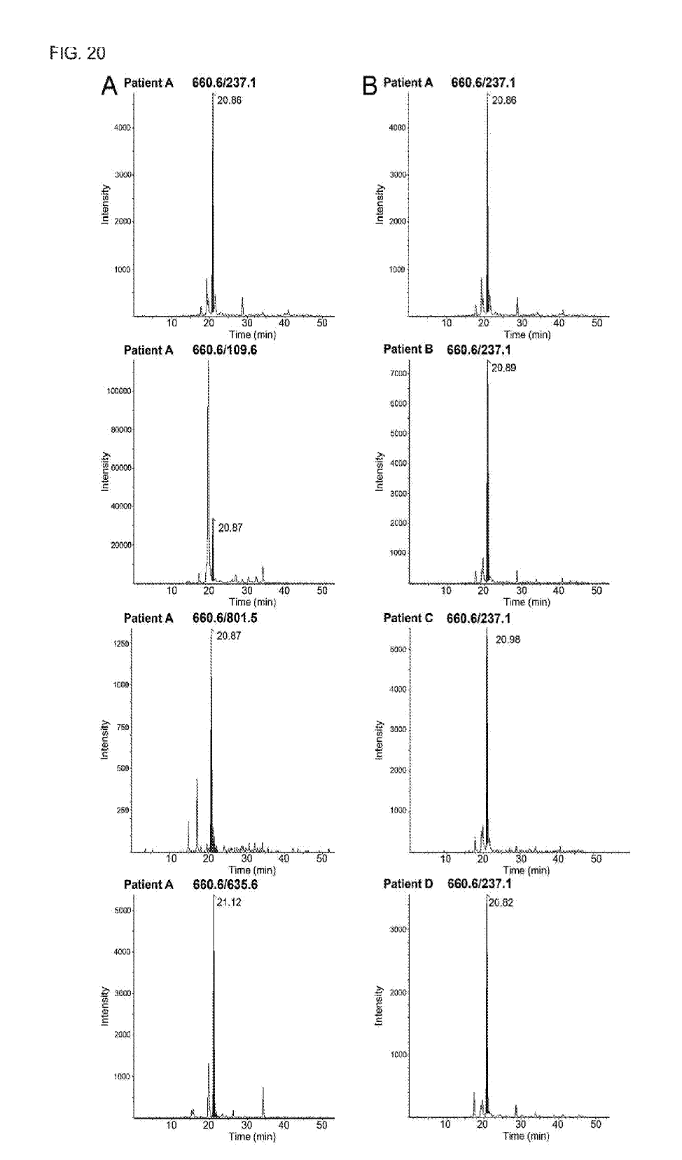

All 12 human plasma samples analyzed, from both diabetic and non-diabetic patients, showed the presence of R257 transitions. Four of the representative human samples, and the presence of transition 660.6/237.1, are shown in FIG. 20. All samples exhibited peaks for R257 transitions, although differences in area under the curve varied between samples. The specificity of four distinct R257 transitions were found to be a reliable indicator of peptide presence (FIG. 20A), and the retention time of the R257 transitions did not vary drastically within each batch of samples analyzed (FIG. 20B). Further quantitative analyses of these transitions to establish potential differences in R257 transition levels between diabetic and non-diabetic individuals are ongoing.

### Example 4 Discussion

Several arginine residues were identified in abundant plasma proteins that are susceptible to MG modification. Most of these arginines were found with both the +54 and +72 modifications. The dihydroxyimidazolidine and open ring intermediates are isobaric, and indistinguishable by mass spectrometry. However, the presence of the +72 adduct further validates these sites as bonafide targets for MG, because arginine must form these structures prior to the subsequent loss of water to generate the +54 adduct. While the use of 500 μM MG is in excess of that observed in human plasma, this concentration was utilized to ensure maximal detection of all sites within the proteome susceptible to any amount of MG. As only select sites were identified, it appears that 500 μM MG did not overwhelm and occupy every arginine site, but rather modified only arginine sites with particular sensitivity. In particular, HSA possesses 27 arginine residues, but only nine were found to be modified by MG. The basis for the selectivity of these nine sites for MG modification is currently under investigation.

Our work has identified numerous preferential sites of MG adduction (Table 11) beyond those previously reported (Baraka-Vidot et al., 2012, Schmidt et al., 2015), and provide a basis to further determine the role such modification play in altered protein function. Thus, it is possible that some of the sites identified within the plasma proteome are critical in terms of function for these particular proteins. Previous work has established that modification of R410 within drug binding site II in HSA results in inhibited ketoprofen binding and esterase activity (Ahmed et al., 2005b). Another important MG-modified arginine identified in HSA was R257, which resides in drug site I. This site was not identified by Ahmed et al. (2005b) possibly due to interferences from multiple overlapping peaks with their LC-MS only approach. Tandem MS/MS is not affected by such interferences, because co-eluting peptides are separated and individually fragmented. Using this approach the R485 MG site in HSA was another site that was uniquely identified. The proximity effect of charged residues in promoting glycation has been proposed (Rabbani and Thornalley, 2012, Venkatraman et al., 2001) and the results herein support and expand upon these findings. Thus, experiments identified several MG modified targets in the plasma proteome in efforts to develop a structure-activity model for MG adduction.

Following identification of sites of modification experiments validated that drug site I in HSA is a target for MG modification, since R218 and R257 were identified as sites for MG modification and both are located in drug site I. The prodan assay was optimized for the shortest time point at which alterations in binding due to MG were detectable, determined as early as 30 minutes. A benefit of using prodan to study drug site I is that, unlike warfarin, the emission peak of the bound prodan is distinct from the emission peak of the free prodan. This property is useful for detecting small changes in free versus bound conformations. In addition to warfarin and prodan, other probes have been used to target drug site I, such as 5-dimethylaminonaphtalene-1-sulfonamide (DNSA), dansylamide, dansyl-L-glutamine, dansyl-L-asparagine, dansyl-L-lysine, n-butyl p-aminobenzoate, and phenol red (Kragh-Hansen et al., 2002). In a displacement assay to study MG, any one of these probes would likely exhibit similar behavior to prodan. The results indicate that arginine residues in drug site I are targets for MG adduction. However, while R218 and R257 reside within the drug site I, modification of the protein was most likely occurring at all five sites identified and changes in tertiary structure affecting protein function due to arginine adduction distal from drug site I cannot be ruled out.

The importance of demonstrating functional changes in HSA due to MG adduction is emphasized by revealing the presence of a MRM transition specific to the R257 peptide in each of the 12 human samples analyzed (FIG. 20). Because this initial human study was not designed to establish absolute levels of MG-modified R257 peptide, but rather its existence (or otherwise) in human plasma, differences between diabetic and non-diabetic patients were not assessed. Work is ongoing to determine potential quantitative differences between R257 (and other modified arginine peptides) MRM transition levels between cohorts.

It is necessary to elucidate the site-specificity of MG adduction, as critical residues involved in protein function could be susceptible to modification. Moreover, particularly reactive MG-binding sites could serve as biomarkers for MG-mediated dysregulation, similar to the use of glycated hemoglobin (HbA1C) as a marker for glucose exposure. Such high-affinity sites of MG adduction may offer a better measure of the in vivo exposure to reactive dicarbonyls. It is equally important to characterize the sites of modification so that specific antibodies can be designed for future high throughput analysis.

TABLE 10
R257 peptide MRM transitions

| R257 Peptide Sequence | Parent | Daughter | CE | Charge | Ion |
|---|---|---|---|---|---|
| VHTEC(57) | 660.6 | 801.5 | 31 | 4 | y20 + 3 |
| C(57)HGDLLEC(57) | | | | | |
| ADDR(54)ADLAK | | | | | |
| | 660.6 | 635.6 | 32 | 4 | y21 + 4 |
| | 660.6 | 237.1 | 35 | 4 | b2 |
| | 660.6 | 109.6 | 90 | 4 | y2 + 2 |
| | 660.6 | 421.5 | 33 | 4 | y7 + 2 |
| | 528.7 | 605.2 | 19 | 5 | b10 + 2 |
| | 528.7 | 594.7 | 19 | 5 | y10 + 2 |
| | 528.7 | 659.3 | 19 | 5 | y11 + 2 |
| | 528.7 | 661.7 | 19 | 5 | b11 + 2 |

Nine transitions were generated for peptide R257 by modifying a pure peptide with MG. The transitions in bold were the transitions selected for further use in analysis, as they had the best linear response to concentration changes in vitro by MG. CE: collision energy.

TABLE 11
Site-specific MG modifications in vitro.

| | | | | MG-HI | MG-DH |
|---|---|---|---|---|---|
| Protein | Accession | Site | Peptide Sequence | (R + 54) | (R + 72) |
| Albumin | P02768 | R81 | LCTVATLRETYGEMADCCAK | X | X |
| | | R117 | LVRPEVDVMCTAFHDNEETFLK[K] | X | X |
| | | R186 | LDELRDEG[K]ASSAK | X | X |
| | | R218 | AWAVARLSQR | X | X |
| | | R257 | VHTECCHGDLLECADDRADLAK | X | X |
| | | R410 | FQNALLVRYTK | X | X |
| | | R428 | [K]VPQVSTPTLVEVSRNLGK | X | X |
| | | R472 | TPVSDRVTK | X | |
| | | R445 | RMPCAEDYLSVVLNQLCVLHEK | X | |
| Serotransferrin | P02787 | R124 | SCHTGLGRSAGWNIPIGLLYCDLPEPR | | X |
| Haptoglobin | P00738 | R41 | LRTEGDGVYTLNDK[K] | X | X |
| | | R243 | VSVNERVMPICLPS[K]DYAEVGR | | X |
| Hemopexin | P02790 | R185 | YYCFQGNQFLRFDPVR | X | |
| Ig lamba-2 | P0CG05 | R83 | SHRSYSCQVTHEGSTVEK | | X |
| chain C regions | | | | | |

Spectra were identified for the R+54 adduct using human plasma or pure protein incubated with 500 μM MG. Spectra containing the intermediate R+72 dihydroimidazolidine adduct are indicated. All cysteines are carbamidomethylated (C+57) prior to digestion. Brackets indicate alternate cleavage peptides detected.

MG-DH: methylglyoxal-derived dihydroxyimidazolidine

MG-HI: methylglyoxal-derived hydroimidazolone

### Example 4 References

Ahmed, N., Babaei-Jadidi, R., Howell, S. K., Beisswenger, P. J., and Thornalley, P. J. (2005a). Degradation products of proteins damaged by glycation, oxidation and nitration in clinical type 1 diabetes. Diabetologia. 48, 1590-1603.
Ahmed, N., Dobler, D., Dean, M., and Thornalley, P. J. (2005b). Peptide mapping identifies hotspot site of modification in human serum albumin by methylglyoxal involved in ligand binding and esterase activity. J. Biol. Chem. 280, 5724-5732.
Ahmed, N. and Thornalley, P. J. (2007). Advanced glycation endproducts: what is their relevance to diabetic complications? Diabetes Obes. Metab. 9, 233-245.
Andon, N. L., Hollingworth, S., Koller, A., Greenland, A. J., Yates, J. R., 3rd, and Haynes, P. A. (2002). Proteomic characterization of wheat amyloplasts using identification of proteins by tandem mass spectrometry. Proteomics. 2, 1156-1168.
Baraka-Vidot, J., Guerin-Dubourg, A., Bourdon, E., and Rondeau, P. (2012). Impaired drug-binding capacities of in vitro and in vivo glycated albumin. Biochimie. 94, 1960-1967.
Beisswenger, P. J., Drummond, K. S., Nelson, R. G., Howell, S. K., Szwergold, B. S., and Mauer, M. (2005). Susceptibility to diabetic nephropathy is related to dicarbonyl and oxidative stress. Diabetes. 54, 3274-3281.
Bose, T., Bhattacherjee, A., Banerjee, S., and Chakraborti, A. S. (2013). Methylglyoxal-induced modifications of hemoglobin: structural and functional characteristics. Arch. Biochem. Biophys. 529, 99-104.
Brock, J. W., Cotham, W. E., Thorpe, S. R., Baynes, J. W., and Ames, J. M. (2007). Detection and identification of arginine modifications on methylglyoxal-modified ribonuclease by mass spectrometric analysis. J. Mass Spectrom. 42, 89-100.
Brownlee, M. (2001). Biochemistry and molecular cell biology of diabetic complications. Nature. 414, 813-820.
Clauser, K. R., Baker, P., and Burlingame, A. L. (1999). Role of accurate mass measurement (+/−10 ppm) in protein identification strategies employing MS or MS/MS and database searching. Anal. Chem. 71, 2871-2882.
Dhar, A., Desai, K., Liu, J., and Wu, L. (2009). Methylglyoxal, protein binding and biological samples: are we getting the true measure? J. Chromatogr. B. Analyt Technol. Biomed. Life. Sci. 877, 1093-1100.
Duran-Jimenez, B., Dobler, D., Moffatt, S., Rabbani, N., Streuli, C. H., Thornalley, P. J., Tomlinson, D. R., and Gardiner, N. J. (2009). Advanced glycation end products in extracellular matrix proteins contribute to the failure of sensory nerve regeneration in diabetes. Diabetes. 58, 2893-2903.
Gallet, X., Charloteaux, B., Thomas, A., and Brasseur, R. (2000). A fast method to predict protein interaction sites from sequences. J. Mol. Biol. 302, 917-926.
Gao, Y. and Wang, Y. (2006). Site-selective modifications of arginine residues in human hemoglobin induced by methylglyoxal. Biochemistry. 45, 15654-15660.
Kall, L., Storey, J. D., MacCoss, M. J., and Noble, W. S. (2008). Posterior error probabilities and false discovery rates: two sides of the same coin. J. Proteome Res. 7, 40-44.
Keller, A., Nesvizhskii, A. I., Kolker, E., and Aebersold, R. (2002). Empirical statistical model to estimate the accuracy of peptide identifications made by MS/MS and database search. Anal. Chem. 74, 5383-5392.
Khuhawar, M. Y., Zardari, L. A., and Laghari, A. J. (2008). Capillary gas chromatographic determination of methylglyoxal from serum of diabetic patients by precolumn derivatization with 1,2-diamonopropane. J. Chromatogr. B. Analyt Technol. Biomed. Life. Sci. 873, 15-19.
Kimzey, M. J., Yassine, H. N., Riepel, B. M., Tsaprailis, G., Monks, T. J., and Lau, S. S. (2011). New site(s) of methylglyoxal-modified human serum albumin, identified by multiple reaction monitoring, alter warfarin binding and prostaglandin metabolism. Chem. Biol. Interact. 192, 122-128.
Kragh-Hansen, U., Chuang, V. T., and Otagiri, M. (2002). Practical aspects of the ligand-binding and enzymatic properties of human serum albumin. Biol. Pharm. Bull. 25, 695-704.
Kuzyk, M. A., Smith, D., Yang, J., Cross, T. J., Jackson, A. M., Hardie, D. B., Anderson, N. L., and Borchers, C. H. (2009). Multiple reaction monitoring-based, multiplexed, absolute quantitation of 45 proteins in human plasma. Mol. Cell. Proteomics. 8, 1860-1877.
Lo, T. W., Westwood, M. E., McLellan, A. C., Selwood, T., and Thornalley, P. J. (1994). Binding and modification of proteins by methylglyoxal under physiological conditions. A kinetic and mechanistic study with N alpha-acetylarginine, N alpha-acetylcysteine, and N alpha-acetyllysine, and bovine serum albumin. J. Biol. Chem. 269, 32299-32305.
Moreno, F. and Gonzalez-Jimenez, J. (1999). Binding of the Promen fluorescent probe to human serum albumin: a fluorescence spectroscopic study. Chem. Biol. Interact. 121, 237-252.
Morgan, P. E., Sheahan, P. J., Pattison, D. I., and Davies, M. J. (2013). Methylglyoxal-induced modification of arginine residues decreases the activity of NADPH-generating enzymes. Free Radic. Biol. Med. 61C, 229-242.
Qian, W. J., Liu, T., Monroe, M. E., Strittmatter, E. F., Jacobs, J. M., Kangas, L. J., Petritis, K., Camp, D. G., 2nd, and Smith, R. D. (2005). Probability-based evaluation of peptide and protein identifications from tandem mass spectrometry and SEQUEST analysis: the human proteome. J. Proteome Res. 4, 53-62.
Rabbani, N. and Thornalley, P. J. (2012). Methylglyoxal, glyoxalase 1 and the dicarbonyl proteome. Amino Acids. 42, 1133-1142.
Schmidt, R., Bohme, D., Singer, D., and Frolov, A. (2015). Specific tandem mass spectrometric detection of AGE-modified arginine residues in peptides. Journal of Mass Spectrometry. 50, 613-624.
Sheetz, M. J. and King, G. L. (2002). Molecular understanding of hyperglycemia's adverse effects for diabetic complications. JAMA. 288, 2579-2588.
Shevchenko, A., Wilm, M., Vorm, O., and Mann, M. (1996). Mass spectrometric sequencing of proteins silver-stained polyacrylamide gels. Anal. Chem. 68, 850-858.
Spivak, M., Weston, J., Bottou, L., Kall, L., and Noble, W. S. (2009). Improvements to the percolator algorithm for Peptide identification from shotgun proteomics data sets. J. Proteome Res. 8, 3737-3745.
Thornalley, P. J. (1993). The glyoxalase system in health and disease. Mol. Aspects Med. 14, 287-371.
Thornalley, P. J., Battah, S., Ahmed, N., Karachalias, N., Agalou, S., Babaei-Jadidi, R., and Dawnay, A. (2003). Quantitative screening of advanced glycation endproducts in cellular and extracellular proteins by tandem mass spectrometry. Biochem. J. 375, 581-592.
Venkatraman, J., Aggarwal, K., and Balaram, P. (2001). Helical peptide models for protein glycation: proximity effects in catalysis of the Amadori rearrangement. Chem. Biol. 8, 611-625.

### Example 5

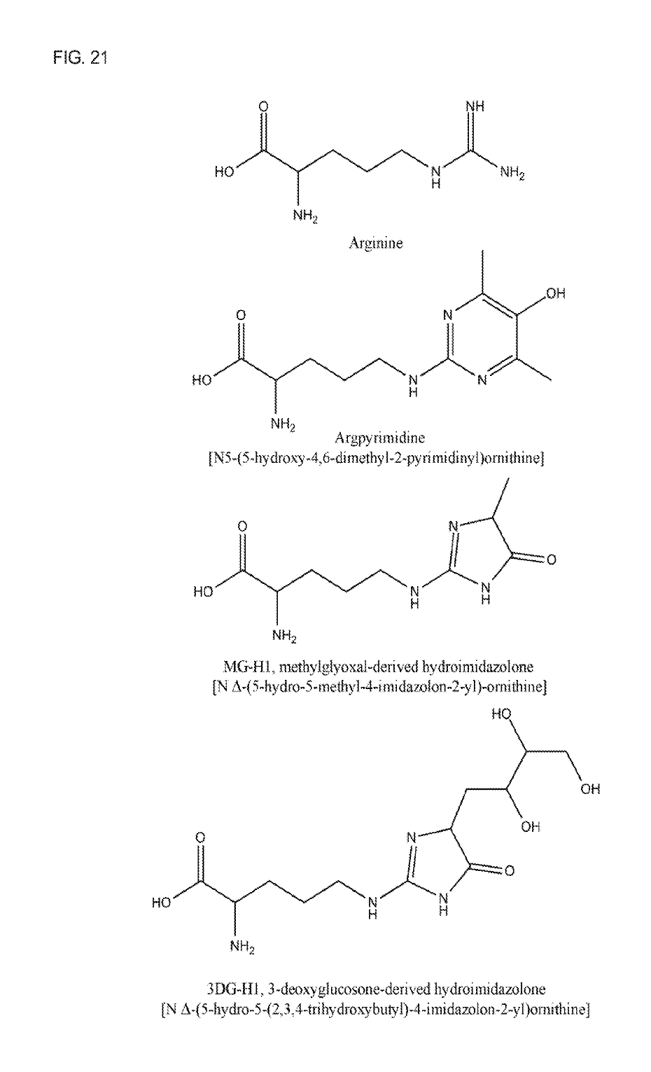

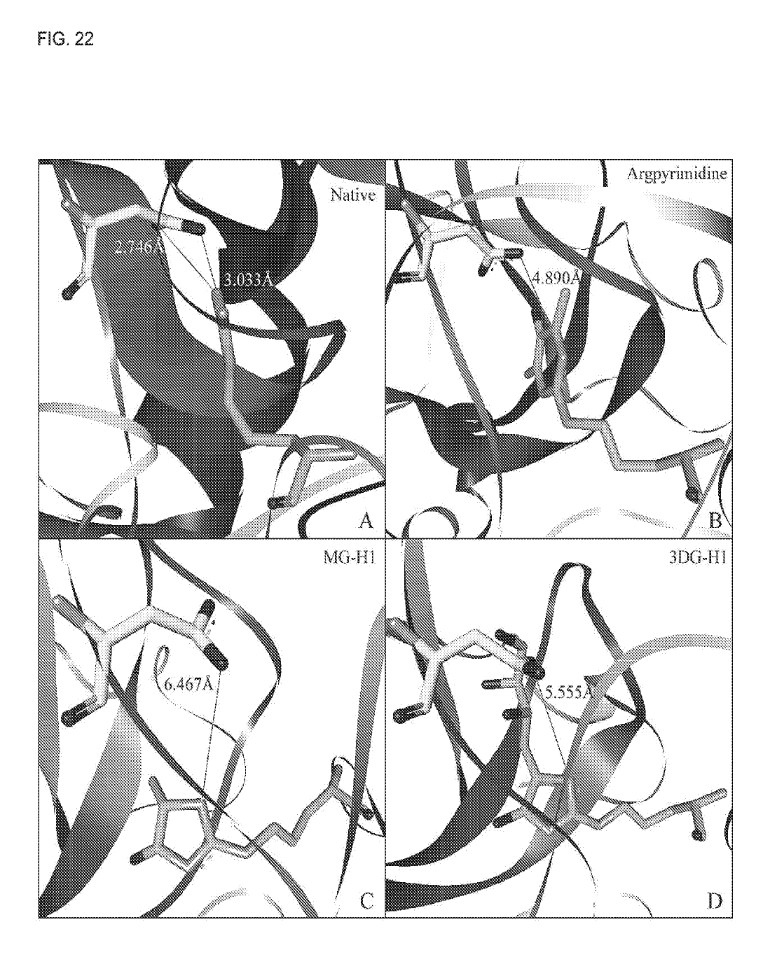

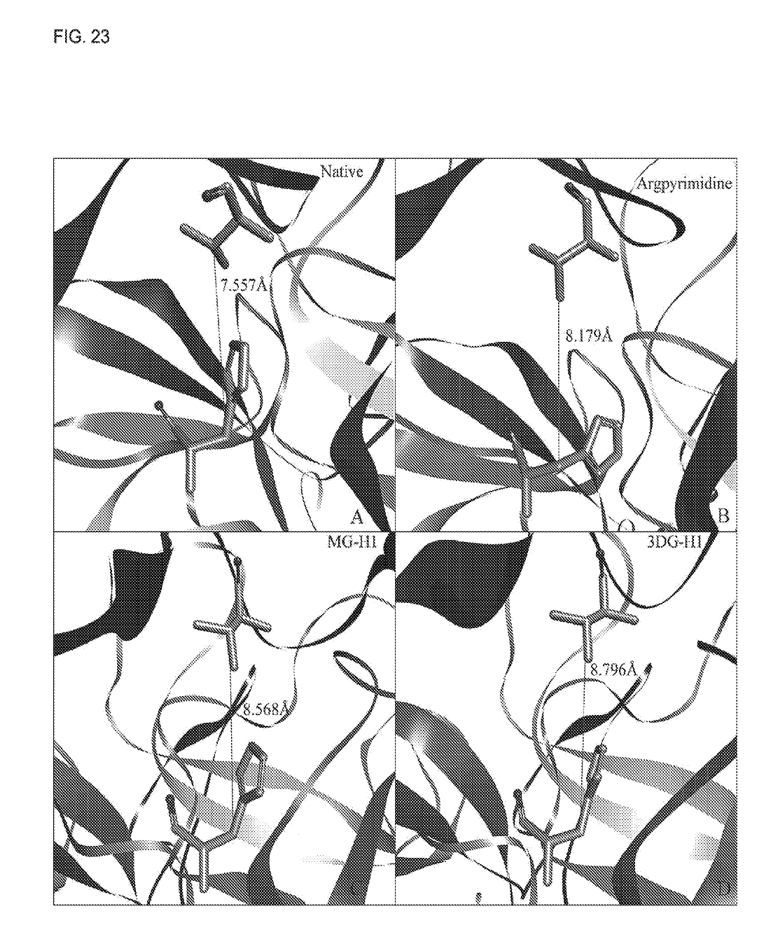

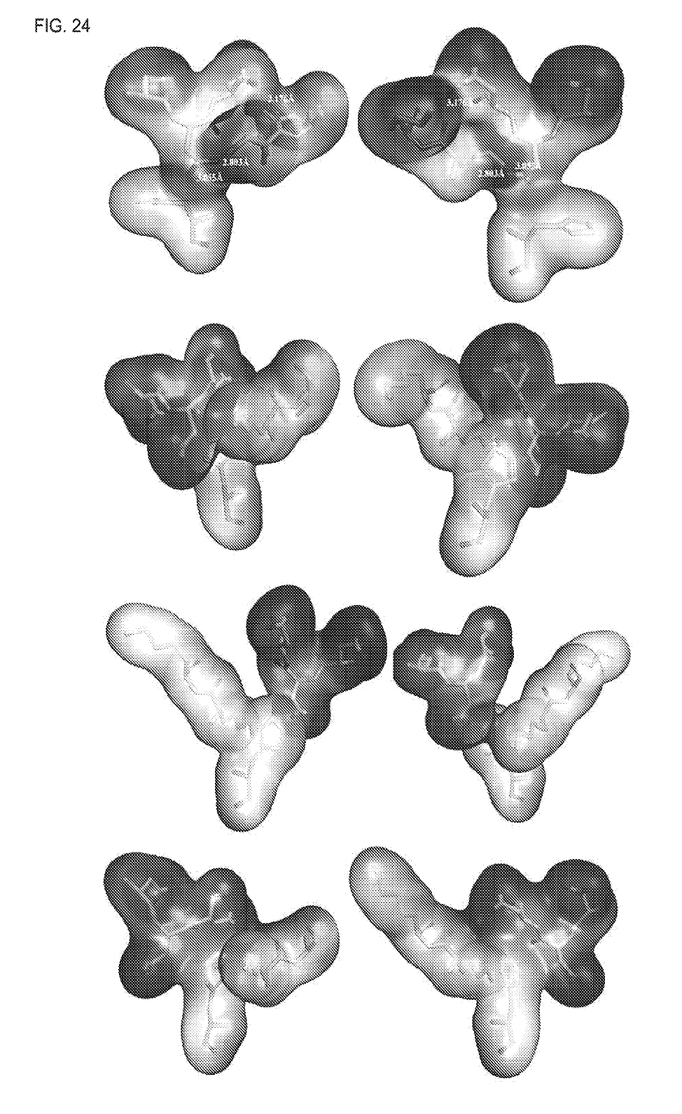

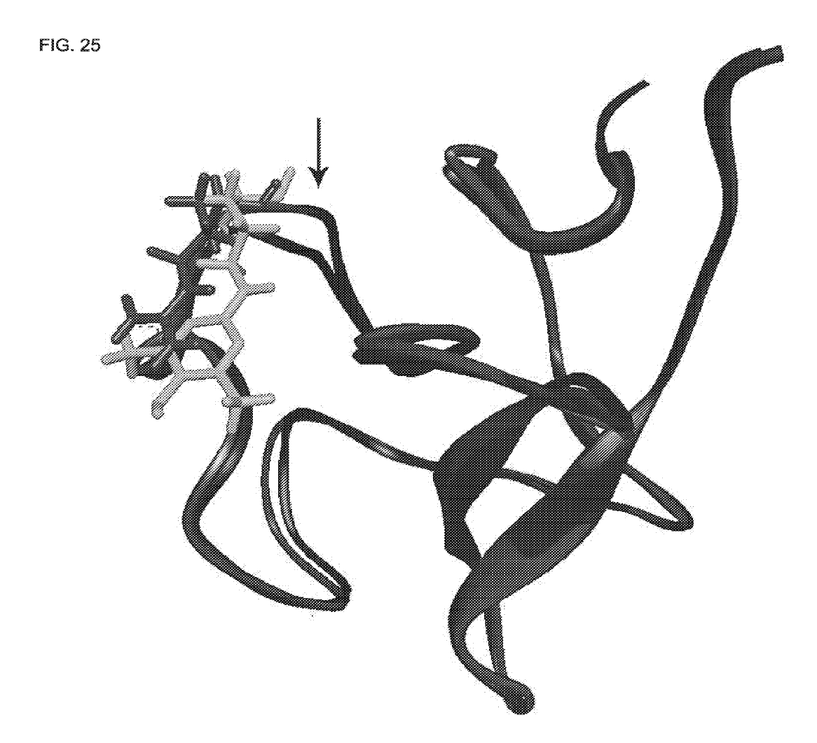

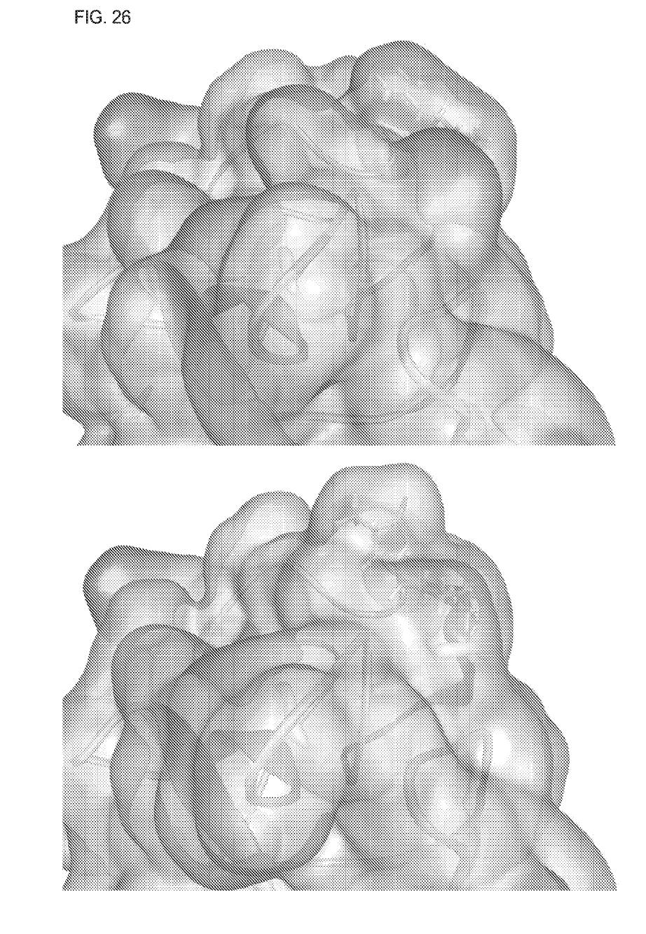

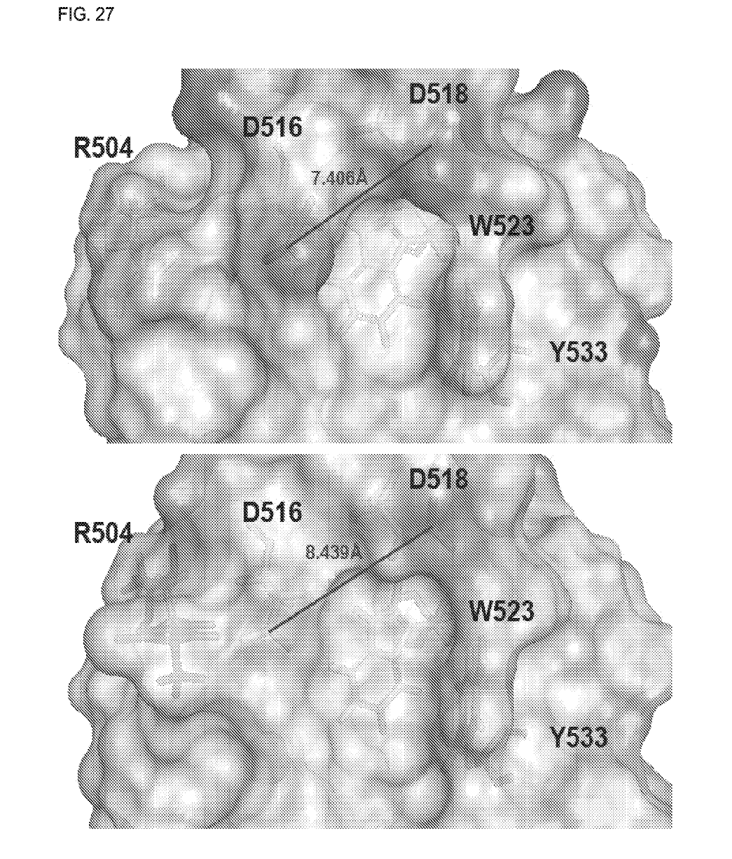

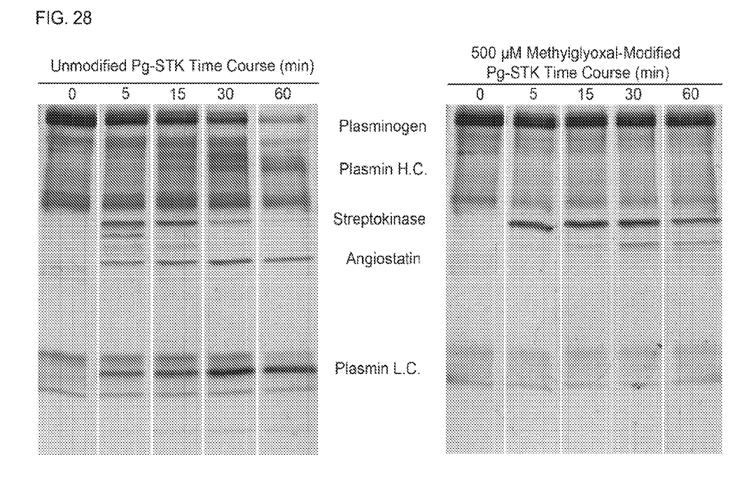

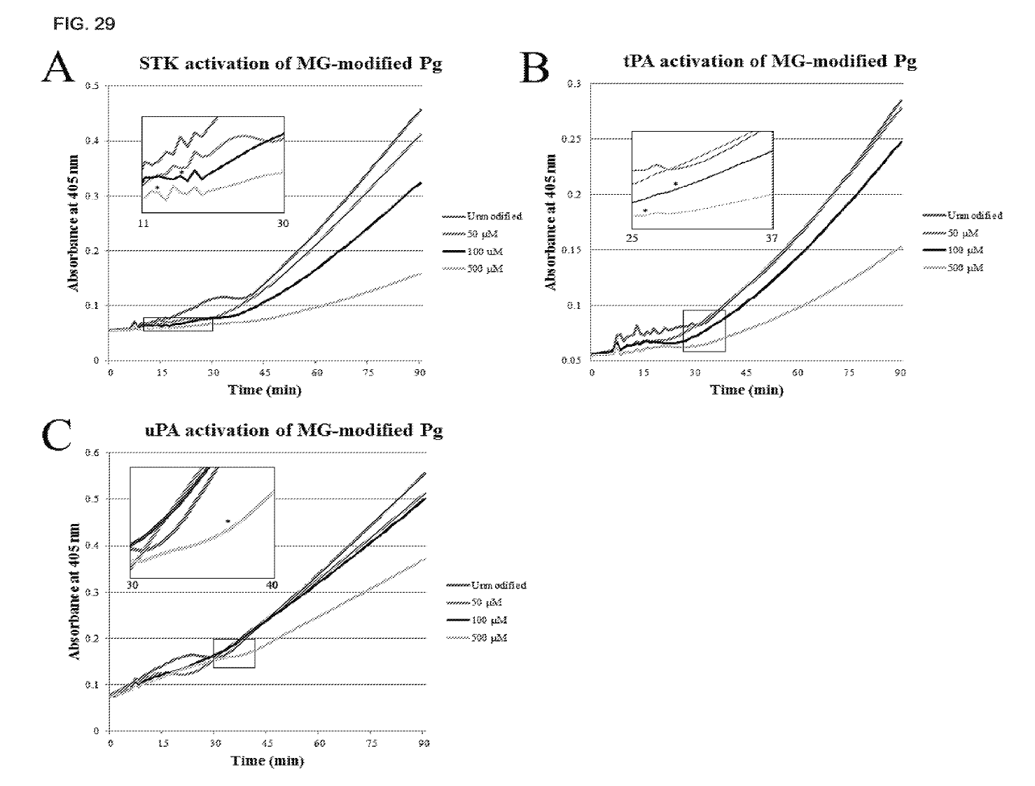

This example demonstrates disruption of the fibrinolysis cascade via dicarbonyl adduction of plasminogen (see, FIGS. 21, 22, 23, 24, 25, 26, 27, 28 and 29).

### Example 5 Materials and Methods

Sequencing grade trypsin was purchased from Promega (Fitchburg, Wis.). 40% MG solution and streptokinase were purchased from Sigma-Aldrich. Glu-plasminogen, fibrinogen, tissue plasminogen activator, urokinase, and chromogenic substrate for Pn were acquired from Molecular Innovations (Novi, Mich.). Thrombin BioUltra was obtained from Sigma Aldrich.

Modification of Pg with qmethylglyoxal

Physiologically relevant concentrations (Chaplen et al., 1998, Ahmed et al., 2002) of MG were utilized for all LC-MS/MS, silver stain and enzyme inhibition analyses of Pg. glu-Pg (10 μg) was incubated with MG (1-500 μM) at 37° C. for 24 Hrs. Samples for STK assay were further processed before being resolved on a 12% SDS-PAGE gel following removal from 37° C. oven.

Gel Excision, Cleanup and Digestion

500 μM MG-modified sample was resolved on a 12% SDS-PAGE gel and gel was stained with coomassie Imperial Protein Stain (Thermo Scientific). Coomassie stained gel-band was excised and washed in ddH₂O for 15 min. H₂O was removed and bands were incubated in 50/50 acetonitrile (ACN):ddH₂O for 15 min. ACN:ddH₂O was removed and bands were incubated with ACN for 5 min. ACN was removed and gels were incubated with 100 mM ammonium bicarbonate (AMBIC). An equal volume of ACN was added to make 1:1 solution and was incubated for 15 min. The ACN/AMBIC solution was removed and remaining gel bands were dried by speed-vacuum. Dithiothreitol (DTT; 10 mM) was added to each band and incubated at 56° C. for 45 min. DTT was removed and sample brought to room temperature (RT). Iodoacetamide (IAA; 55 mM) was added to each sample and incubated at RT for 30 min in dark. IAA was removed and 100 mM AMBIC was added and bands were incubated for 5 min. An equal volume of ACN was added to make a 1:1 solution and incubated for 15 min. The ACN/AMBIC solution was removed and bands were dried by speed-vacuum. Bands were digested with sequencing-grade trypsin (Promega; 400 ng/band) in 50 mM AMBIC and incubated on ice for 45 min. Tryptic solution was removed and 50 mM AMBIC was added to each band and incubated overnight at 37° C. to complete digestion. Digests were acidified using 10% trifluoroacetic acid (TFA). Supernatant was saved. Band was covered with TFA:ACN (0.1%:60%) and sonicated at 20° C. water bath for 30 min. Supernatant was combined with previous fraction. Samples were speed vacuumed to a final volume of 10 μL prior to LC-MS/MS analysis on an LTQ Orbitrap Velos mass spectrometer as described below.

Tandem Mass Spectrometry Coupled to Liquid Chromatography (LC-MS/MS)

LC-MS/MS analysis of in-gel trypsin digested-proteins (Shevchenko et al., 1996) was carried out using a LTQ Orbitrap Velos mass spectrometer (Thermo Fisher Scientific, San Jose, Calif.) equipped with an Advion nanomate ESI source (Advion, Ithaca, N.Y.), following ZipTip (Millipore, Billerica, Mass.) C18 sample clean-up according to the manufacturer's instructions. Peptides were eluted from a C18 precolumn (100-μm id×2 cm, Thermo Fisher Scientific) onto an analytical column (75-μm ID×10 cm, C18, Thermo Fisher Scientific) using a 5-20% gradient of solvent B (acetonitrile, 0.1% formic acid) over 65 minutes, followed by a 20-35% gradient of solvent B over 25 minutes, all at a flow rate of 400 nl/min. Solvent A consisted of water and 0.1% formic acid. Data dependent scanning was performed by the Xcalibur v 2.1.0 software (Andon et al., 2002) using a survey mass scan at 60,000 resolution in the Orbitrap analyzer scanning m/z 350-1600, followed by collision-induced dissociation (CID) tandem mass spectrometry (MS/MS) of the fourteen most intense ions in the linear ion trap analyzer. For human samples analyzed, an inclusion list was utilized that first preferentially allowed the ions corresponding to known modified peptides to undergo CID prior to that of the most intense ions. Precursor ions were selected by the monoisotopic precursor selection (MIPS) setting with selection or rejection of ions held to a +/−10 ppm window. Dynamic exclusion was set to place any selected m/z on an exclusion list for 45 seconds after a single MS/MS. All MS/MS samples were analyzed using Sequest (Thermo Fisher Scientific, San Jose, Calif., USA; version 1.3.0.339). Sequest was set up to search human proteins downloaded from UniProtKB on Aug. 6, 2013. Variable modifications considered during the search included methionine oxidation (15.995 Da), cysteine carbamidomethylation (57.021 Da), as well as two products of the adduction of arginine residues by MG, MG-H1 (54.011 Da) and MG-DH (72.021 Da). At the time of the search, the Human UniProt database contained 88,323 entries. Proteins were identified at 95% confidence with XCorr scores (Qian et al., 2005) as determined by a reversed database search using the Percolator algorithm (http://per-colator.com) (Spivak et al., 2009). Identified modified peptides were considered with a q-value<0.01 (Kall et al., 2008).

Molecular Modeling of Pg and tPA Complex

The X-ray crystal structure coordinates for human Pg catalytic domain (PDB code: 1ddj) (Wang et al., 2000) and human tPA (PDB code: 1bda) (Renatus et al., 1997) were used for modeling of the complex structure. Model for the complex structure was created using published data on Pg-tPA interaction (Wang et al., 2000). Charges were assigned using consistent valence force field (CVFF) parameters within Insight II 2005L modeling software (Accelrys, Inc., San Diego, Calif.). This complex was then soaked in TIP3P water molecules. This assembly was then subjected to molecular minimization and dynamics protocol as elaborated below. The assembly was first subjected to 100000 steps of minimization using Discover 3.0 and then dynamic equilibration for 50 picoseconds (ps) and simulations for 450 ps. Trajectories were collected every 1.0 ps. Trajectory frames were analyzed using their potential energy values and twenty lowest potential energy structures were used to create the average structure. This average structure was minimized using 100000 steps of minimization. The final minimized structure was then used for the analysis, and the interaction energy values were calculated using Affinity docking module.

Three adducts (MG-H1, [N Δ-(5-hydro-5-methyl-4-imidazolon-2-yl)-ornithine]; 3DG-H1, [N Δ-(5-hydro-5-(2,3,4-trihydroxybutyl)-4-imidazolon-2-yl)-ornithine]; and argpyrimidine [N5-(5-hydroxy-4,6-dimethyl-2-pyrimidinyl)ornithine]) were built on the R561 residue of Pg using Insight II—builder module. Molecular minimization and dynamics protocol was repeated for adducted Pg-tPA complexes as described earlier.

Molecular Modeling of Kringle 5

The X-ray crystal structure coordinates for human kringle (KR) 5 domain (PDB code: 2KNF) (Battistel et al., 2009) were used to build the models of the adducts (MG-H1 [N Δ-(5-hydro-5-methyl-4-imidazolon-2-yl)-ornithine]; and argpyrimidine [N5-(5-hydroxy-4,6-dimethyl-2-pyrimidinyl)ornithine]). Molecular modeling studies were performed using Sybyl 8.0 software available from Tripos Inc. Appropriate chemical adducts were created and the charges were assigned using Gasteiger-Huckel parameters within Sybyl. The adduct structure was then subjected to molecular minimization using 10000 steps of minimization. The refined structure was then used for the analysis.

Reaction of Pg with Activating Enzymes.

Upon removal from the 37° C. oven, Pg was buffer exchanged in a 3-kDa spin-filter (Amicon YM-3; Millipore)) to ensure the removal of MG from the sample. Following centrifugation, the volume of each sample was brought to 150 μL, and divided into five 30 μL samples for different incubations. STK (3U), tPA (100 nM) or uPA (100 nM) was used per sample. STK, tPA or uPA was incubated with Pg for 0, 5, 15, 30, 60, or 90 min. At the conclusion of the enzyme incubation, samples were denatured with 5% BME and heated for 5 minutes at 100° C. on a heat block prior to being resolved on a 12% SDS-PAGE gel.

Silver Staining

Gels were stained using a modified version of the method from (Blum et al., 1987). Gel was fixed overnight in a solution of 50% MeOH, 12% glacial acetic acid, and 0.0185% formaldehyde. The gel was washed 2×20 min with 50% EtOH followed by 20 minutes with 30% EtOH. The gel was subjected to a pretreatment solution of 0.2 mg/ml of sodium thiosulfate (Na₂S₂O₃) for 1 minute and was rinsed 3×20 sec with MilliQ H₂O. The gel was impregnated with the silver solution (2 mg/ml silver nitrate [AgNO₃], 0.078% formaldehyde) for 20 minutes. Following two 20-second MilliQ H₂O washes, the gel was immediately developed with a solution of 60 mg/ml Na₂CO₃, 4 μg/ml sodium thiosulfate, and 0.0185% formaldehyde until protein bands were visible. Upon acceptable staining progression, the gel was rinsed twice for 2 min with MilliQ H₂O before the reaction was stopped with a solution of 50% MeOH and 12% glacial acetic acid. Gel was washed and stored in 50% MeOH. Gel was imaged with a ChemiDoc XRS System (BioRad).

Chromogenic Activity Assay

Pg (10 μg) was incubated with MG (50-500 μM) in 10 mM HEPES/150 mM NaCl (pH 7.4) for 24 hours at 37° C. Following modification, sample volumes were increased to 150 μL and placed in 5 kDa spin filters to reduce volume and remove excess MG from solution. A 96-well plate was incubated with 200 μL of 260 nM fibrinogen in TBS for 40 min. at room temperature. Fibrinogen solution was removed and replaced with 250 μL TBS containing 3% bovine serum albumin and 0.01% TWEEN for 90 minutes at 37° C. Blocking solution was removed and the plate was washed twice with solution of 50 mM Tris-HCl, 110 mM NaCl and 0.01% TWEEN (pH 7.4). Thrombin (5 units/mL) and 5 mM CaCl₂ in 50 mM Tris-HCl, 110 mM NaCl (pH 7.4) was added to the wells for 45 min. A high salt wash (1M NaCl, 50 mM Tris-HCl, pH 7.4) followed by a TBS-TWEEN wash completed plate prep. Into each well, 198 μL of 10 mM HEPES/150 mM NaCl (pH 7.4) and 2 μL of 25 mM chromogenic substrate for Pn (D-VLK-pNA) were added. To appropriate wells were added 2 μL of 20 μM Pg, either a 24-hour unmodified control or 24 hour 50-500 μM MG modified. Activators were added to respective wells to test activation of unmodified and modified Pg. Each activator enzyme (2 μL) was utilized in appropriate wells for tPA (12 nM), uPA (50 nM) and STK (5 nM) (Ajjan et al., 2013, Zhang et al., 2012). Following enzyme addition, plate was immediately read on a SpectraMax M2 plate reader (Molecular Devices) every minute for 90 minutes at an absorbance of 405 nm.

### Example 5 Results

Identification of Sites of MG-Adduction of Plasminogen

Utilizing 500 μM MG modification of glu-Pg, and subsequent LC-MS/MS analysis of the sample by LTQ Orbitrap Velos, specific sites of arginine adduction by MG were determined. A large number of arginine sites were found to be modified, in most cases with both MG-H1 and MG-DH adducts present. The sites of modification are visualized, and the list of modifications can be found in Table 12. A q-value below 0.01 and an XCorr score greater than 2.0 were utilized as thresholds for modified peptide identification. A q-value is a false discovery rate analogue to the p-value. Many of the q-values were zero, indicating a near statistical impossibility that these spectra were false positives. An XCorr score is a value that is assigned based on how well the spectra correlates with a given peptide, with a value of 2 traditionally indicative of a good correlation. The most commonly identified site was the arginine at position 504, as 16 spectra for the MG-H1 adduct were observed at this position. R530 and R561 were also highly identified, both with 12 spectra identifying the MG-H1 version of the respective peptides. In total, 30 of the 42 arginine residues of the protein were observed to be modified to some extent. R504, R530, and R561 were selected for further molecular modeling analysis, both due to the possibility that they are more highly modified than other sites, and that they are in regions of the protein involved in critical functions of Pg. R504 and R530 are located in the KR5 domain of the protein, responsible for binding to lysine residues of fibrin and thus integration of Pg into the backbone of a clot. R561 is the point of cleavage for Pg into Pn, and adduction at this residue could prevent this cleavage.

Modeling Dicarbonyl Adducts at Site R561 of Plasminogen

Due to the large size (˜100 kDa) of Pg, a full crystal structure for the protein did not exist until very recently. However, separate domains of the protein have been crystallized and their tertiary structure determined (Peisach et al., 1999). The R561 containing catalytic unit of Pg is one of the domains that has a crystal structure available. The interaction between this catalytic unit and tPA was reproduced from previous work by (Wang et al., 2000). From this native model, arginine adducts were modeled in place of unmodified arginine at position 561 due to its detection by both MS techniques and importance to Pg cleavage. The lowest energy conformation was adopted for each modified model, which included MG-H1, MG-DH, argpyrimidine, and 3DG-H1.

Interaction energy changes are considered substantial enough to change protein binding to its partner at >2.5 kcal/mol (Ofran and Rost, 2007). Argpyrimidine modification caused the largest change in interaction energy from the native model, with a 410.4 kcal/mol weaker interaction between Pg and tPA. MG-H1 caused a decrease in interaction energy of 225.6 kcal/mol from the native model. 3DG-H1 caused a decrease in interaction energy of 232.2 kcal/mol from the native model. The difference in interaction energy came almost entirely from the electrostatic alterations, with steric changes having little effect on the overall interaction energy (Table 13).

We examined changes in the models at the amino acid level based on important published residue interactions between Pg and tPA. In the native model, R561 of Pg forms a salt bridge with aspartic acid-189 (D189) of tPA at a distance of 3.894 Å. In the argpyrimidine model, this salt bridge cannot occur due to the charge loss associated with ring formation, and the increased distance due to steric effects. If the salt bridge was possible, the distance increased to 5.486 Å, further than the 4 Å salt bridge limit. MG-H1 and 3DG-H1 models produced similar results. In addition, valine-562 (V562) of Pg is known to be in contact with catalytic histidine-57 (H57) of tPA. Contact is defined as a <8 Å distance between the β-carbon of the amino acid residue (Olmea and Valencia, 1997). The native model indicates a V562-H57 distance of 7.557 Å. In all three modified models, the V562-H57 distance is longer than the 8 Å contact distance. Further, it is believed that the interaction between the negatively charged cluster of residues D95-97 of tPA have an important interaction with basic cluster of residues lysine-556 (K556), K557 and H569 of Pg. In the native model, there are three possible salt bridges: K557-D97, and the availability of two bridges with K556-D96. In all three modified models, no salt bridges exist between these six amino acids in the two clusters, and the modeled interpolated surfaces display a shift towards acidity in all three models.

Modeling Dicarbonyl Adducts at Sites R504 and R530 on Plasminogen

For R504 and R530, the KR5 domain crystal structure (PDB: 2KNF) was utilized to study possible effects of adduction at these residues (Battistel et al., 2009). Both MG-H1 and argpyrimidine were modeled in place of arginine at these residues. Neither residue is known to be directly responsible for the lysine binding function of KR5, but they are proximate to residues that are, and overall structural changes as well as binding pocket structural changes were explored.

R504 had the most observable tertiary structure changes, and these changes were detected both due to MG-H1 and argpyrimidine modification. The most drastic change was to the C-terminal side of this modification, as the modification caused what looked to be an alpha-helix type fold, a departure from the normal folding. Argpyrimidine modification at the same site indicates a similar folding change.

Adduction by MG at site R530 indicated that MG-H1 modification had little effect on overall tertiary structure of the KR5 domain, perhaps due to the fact that the residue is oriented entirely outwards from the molecule, negating the steric and charge difference. Both MG-H1 and argpyrimidine (data not shown) exhibited similar results at this residue.

Overall charge, as displayed by the interpolated charge surface, was unsurprisingly altered due to modification at both R504. There was a loss of basicity due to the loss of charge at the residue, and in particular with R504, the area surrounding the residue was altered as well, with an overall effect of neutralization of the charge. Change of charge state at R530 due to modification was also observed, though the area surrounding the residue was unaffected.

The primary ligand docking site for the KR5 domain of Pg has been identified, and the critical residues for this include aspartic acids (D) 516 and 518, typtophan (W) 523 and tyrosine (Y) 533 (Battistel et al., 2009). Key to the binding of lysine residues on fibrin, and the ligand trans-4-(aminomethyl)cyclohexanecarboxylic acid (AMCHA; an antifibrinolytic known to competitively inhibit activation of plasminogen into plasmin) is the CY carboxylate distance of D516 and D518, ˜7.406 Å in the KR5 crystal structure. A CY carboxylate distance between this anionic pair greater than 8.15 Å was non-conducive to ligand binding. MG-H1 adduction at site R504 causes the D516/D518 CY carboxylate distance to increase to 8.439 Å, indicating a likely reduction in ligand affinity. The overall binding pocket can be observed changed as well, as the overall pocket is wider and the carboxylate of D516 internalized, reducing surface charge and again possibly affecting ligand binding. This change was observed most greatly with MG-H1 binding at R504, though argpyrimidine exhibited a similar change (data not shown).

Breakdown of MG-Modified Pg from STK

In order to better study plasminogen breakdown into its various resulting cleaved products, silver staining was selected as a sensitive and total protein stain. All three activating enzymes were inhibited by MG-modification to varying degrees.

Activation of Pg by STK was inhibited by MG. The cleavage products at molecular weights consistent with Pn heavy chain, Pn light chain, and angiostatin were observed to increase in the control gel, but not in the MG-treated gel. Additionally, a drop in Pg was observed over time in the control gel, indicating that the protein was continuously being activated over the 60 minutes assay. The Pg band remains constant over 60 minutes in the modified gel, however, indicating a lack of activation. A band consistent with STK molecular weight also appears to decrease over time, indicating that STK may be forming its activator complex with Pg over time (Loy et al., 2001). This complex was not observed, but is known to form, and offers an explanation for the STK decrease over time. In the modified gel, no decrease in this same band is observed, indicating that the activator complex does not form.

Both the tPA and uPA were more potent activators of Pg, and thus a higher concentration of MG was required to modify Pg to a point where activation of the zymogen was affected. In both cases, tPA and uPA display Pn heavy and light chain bands in the unmodified gel, and lesser amounts of these same proteins in the MG-modified gel. Additionally, the latest time point was extended to 90 minutes to better observe changes in activation.

Inhibition of Plasmin Generation by MG Modification.

In order to assess a broader overall effect of MG modification on Pg activation to Pn, an assay utilizing a chromogenic substrate was performed. The chromogenic substrate is cleaved in the presence of Pn, releasing a compound that is detectable by measuring absorbance at 405 nm. Because no Pn was added to the samples, all of the Pn was produced by activation of Pg. Three activators, tPA, uPA, and STK, were assessed regarding their ability to generate Pn from modified and unmodified Pg in the presence of a fibrin matrix.

The assay indicated that with all three enzymes, MG modification of Pg for 24 hours led to a delay in activation of the protein. STK was the activator most affected by MG-modification, exhibiting a delay in activation of Pg by 16 minutes for 100 μM MG-modified Pg and 12 minutes for 500 μM MG-modified Pg. Activation of MG-modified Pg by tPA was significantly delayed (p<0.05) by 30 minutes for 100 μM MG-modified Pg and 28 minutes for 500 μM MG-modified Pg when compared against tPA activation of unmodified Pg. Activation of MG-modified Pg by uPA was less profound, only exhibiting a significant delay in the 500 μM MG-modified Pg detectable beginning at 37 minutes.

### Example 5 Discussion

The mechanisms by which AGE contribute to diabetic complications, especially CVD, remain unclear. MS-based approaches were developed to identify MG-modified proteins and computer modeling coupled to in vitro functional assays to interrogate the consequences of such structural protein modifications. Arginine reacts with dicarbonyls, forming stable ring structures, including MG-H1, MG-DH, and argpyrimidine. This adduction results in a loss of positive charge, as arginine is positively charged at physiological pH, and the ring structures are uncharged. The change of charge status may have important consequences on the physiological function of critical arginine sites in protein-protein or DNA-protein interactions. When adding potential modifications to database searches, false positives can result, and it is necessary to manually validate MG-modified spectra by using criteria specific for this modification. Operating under the presumption that trypsin does not cleave at MG-modified arginines, any results that gave a sequence with a C-terminal modified arginine were considered false positives. Using these standards, 30 sites were identified by LC-MS/MS, with the highest amount of adduction, both with MG-DH and MG-H1 detected at R504, R530 and R561. Argpyrimidine adducts (R+80) are known to occur in vivo, but at 22 times lower concentrations than MG-H1 (Ahmed et al., 2003), and were therefore not searched for in the mass spectrometry studies.

AGEs are known to be deleterious to proteins, so theoretical modeling was performed to determine the effect of single-site arginine modification on the Pg-tPA interaction. The molecular model of the Pg/tPA interaction revealed exactly how important R561 is to the function of Pg. The profound decrease in the Pg/tPA interaction energy due to the creation of an adduct at R561 was somewhat unexpected. In an interaction that encompasses 512 residues in the model (56,320 Da, based on an average amino acid weight of 110 Da), an 80 Da addition (argpyrimidine) to a single arginine caused a decrease of 410.4 kcal/mol. This one adduction results in a remarkable energy change that is 164-fold higher than the minimum energy change required to cause an altered interaction. In addition, the two hydroimidazolone adducts created a decrease in energy that was more than 90-fold greater than the minimum 2.5 kcal/mol change required to change protein-protein interactions. These drastic changes clearly point to R561 as one of the most critical residue interactions between Pg and tPA. D97 of tPA as well as K556 and K557 of Pg are highly conserved among all species, and believed to be extremely important in regards to plasminogen activation (Zhang et al., 1997). Critical salt bridges at these residues, and others that assist in creating the correct tertiary structure for R561/V562 cleavage, no longer form when R561 is modified, which in turn likely prevents cleavage from occurring. Without cleavage, this would lead to a decrease in plasmin concentration and a decrease in fibrinolysis, ultimately leading to altered hemostasis towards increased clotting.

Similar to the effects observed by modeling MG adducts at R561, modeling onto R504 utilizing the KR5 domain crystal structure indicated that functional impairment was possible due to this single modification. Importantly, the altered charge and steric effects caused by both MG-H1 and argpyrimidine modification at R504 appeared to drastically alter the surrounding areas of the molecule, with an overall neutralization observed. Additionally, the ligand binding site of the domain was also modified, despite R504 not being directly involved in this region of the molecule. The important D516/518 CY carboxylate distance was increased by over 1 Å due to MG-H1 modification, a change which implies impaired ligand binding (Battistel et al., 2009). Moreover, the overall binding pocket was altered by R504 MG-H1 modification, with the pocket becoming wider and the charge of the carboxylate groups of D516 negated due to altered orientation of the residue. These changes indicate that although R504 is not involved directly in the lysine-binding function of the kringle 5 domain, modification of this residue may reduce affinity for the lysine-residues of the fibrin backbone of a clot, and affect overall incorporation of Pg into a clot.

Pg is traditionally degraded by tPA in vivo. However STK is used pharmacologically as a thrombolytic agent to acutely break down blood clots by quickly degrading Pg to plasmin (Young et al., 1998), with peak effects taking approximately 15 min. STK, incubated with Pg, should cause a decrease in Pg protein concentration, as observed by silver stain at 100 kDa with a concomitant increase in Pg breakdown products at less than 100 kDa with the appearance of lower bands consistent with the size of Pn heavy and light chain. The results indicated that the normal breakdown of Pg is impaired due to adduction by MG, agreeing with similar studies performed for shorter time periods at higher concentrations of MG (Lerant et al., 2000). Similar effects were observed with the endogenous activators, as 24-hr MG exposure clearly affected activation of Pg by tPA and uPA. Although the studies with tPA and uPA required elevated levels of MG to observe an effect, it was an effective qualitative study to determine which breakdown products could be inhibited from forming with MG. Further quantitative studies using lower MG concentrations occurred with the kinetic assay.

The similar activation studies, which allowed for a more in depth examination of altered kinetics, indicated that all three activator's effects are inhibited, with the inhibition being both time and concentration dependent. While full inhibition is not reached, what is clear is that MG-exposure causes a significant delay in activation of Pg by each of the enzymes. When these molecules (STK and tPA) are used in the clinic, the desire is for them to take effect in a matter of minutes and not hours. Any delay caused by a less functional version of the protein could affect efficacy of these therapies in T2DM patients with elevated MG levels.

It is clear that STK mediated activation of glu-Pg is decreased following adduction with MG, most probably due to R561 or R504 adduction. Additional studies to identify specific R sites which preferentially impact the loss of Pg function are warranted. The decrease in activity of STK is consistent with clinical data showing that the effectiveness of STK in treating acute myocardial infarction is less effective in patients with T2DM (Chowdhury et al., 2008). MS offers the best approach for determining the overall contribution of site-specific adduction to disrupted hemostasis in T2DM (Kimzey et al., 2011). Application of MS-based identification of MG-Pg adducts will be a critical aid in determining whether MG-modified Pg could be utilized as a functional biomarker to predict diabetic CVD complications.

TABLE 12
LC-MS/MS identified sites of MG-modification of plasminogen.

| | | Spectra | Modifi- | | | MH + | RT |
|---|---|---|---|---|---|---|---|
| Site | Peptide Sequence | Observed | cations | XCorr | Charge | [Da] | [min] |
| 43 | C(+57)EEDEEFTC(+57) | 1 | MG-HI | 2.92135 | 3 | 2289.909 | 24.03564 |
| | RAFQYHSK | | | | | | |
| 61 | EQQC(+57)VIMAENRK | 4 | MG-HI | 2.955172 | 2 | 1559.73 | 20.0872 |
| 61 | EQQC(+57)VIMAENRK | 3 | MG-DH | 2.679394 | 2 | 1577.74 | 18.20813 |
| 68 | SSIIIRMR | 2 | MG-DH | 2.730202 | 2 | 1047.596 | 23.10083 |
| 68 | KSSIIIRMR | 1 | MG-HI | 2.592713 | 2 | 1157.682 | 20.88797 |
| 70 | MRDVVLFEK | 7 | MG-DH | 3.285292 | 3 | 1208.634 | 27.57829 |
| 70 | MRDVVLFEKK | 4 | MG-HI | 4.433904 | 3 | 1318.717 | 23.58504 |
| 70 | M(+16)RDVVLFEK | 4 | MG-HI | 3.295085 | 3 | 1206.619 | 24.63987 |
| 70 | MRDVVLFEK | 4 | MG-HI | 3.152577 | 2 | 1190.62 | 25.31826 |
| 70 | MRDVVLFEKK | 2 | MG-DH | 3.73222 | 3 | 1336.727 | 22.97411 |
| 70 | M(+16)RDVVLFEK | 2 | MG-DH | 2.819363 | 3 | 1224.627 | 23.83258 |
| 115 | WSST SPHRPR | 4 | MG-HI | 2.644886 | 2 | 1264.612 | 14.33063 |
| 117 | WSST | 9 | MG-HI | 5.022877 | 3 | 3239.473 | 22.70005 |
| | SPHRPRFSPATHPSEGLEENYC | | | | | | |
| | (+57)R | | | | | | |
| 117 | WSST | 9 | MG-DH | 3.155099 | 4 | 3257.479 | 23.37743 |
| | SPHRPRFSPATHPSEGLEENYC | | | | | | |
| | (+57)R | | | | | | |
| 115/ | WSST | 3 | MG-HI; | 4.054143 | 4 | 3293.481 | 23.2631 |
| 117 | SPHRPRFSPATHPSEGLEENYC | | MG-HI | | | | |
| | (+57)R | | | | | | |
| 115/ | WSST | 2 | MG-DH; | 2.584404 | 4 | 3311.488 | 24.1241 |
| 117 | SPHRPRFSPATHPSEGLEENYC | | MG-HI | | | | |
| | (+57)R | | | | | | |
| 115/ | WSST | 1 | MG-DH; | 2.458603 | 4 | 3329.508 | 24.63564 |
| 117 | SPHRPRFSPATHPSEGLEENYC | | MG-DH | | | | |
| | (+57)R | | | | | | |
| 134 | FSPATHPSEGLEENYCRNPDNDPQ | 1 | MG-DH | 3.64746 | 4 | 4279.85 | 26.47397 |
| | GPWC(+57)YTTDPEKR | | | | | | |
| 220 | NPDRELRPWC(+57)FTTDPNKR | 6 | MG-DH | 3.82446 | 4 | 2374.134 | 25.69231 |
| 220 | NPDRELRPWC(+57)FTTDPNK | 5 | MG-HI | 2.874007 | 4 | 2200.024 | 26.77613 |
| 220 | NPDRELRPWC(+57)FTTDPNK | 1 | MG-DH | 2.277936 | 4 | 2218.033 | 27.48596 |
| 223 | NPDRELRPWC(+57)FTTDPNKR | 11 | MG-HI | 4.081222 | 3 | 2356.125 | 26.91059 |
| 223 | ELRPWC(+57)FTTDPNKR | 4 | MG-HI | 2.733854 | 3 | 1873.903 | 26.14231 |
| 220/ | NPDRELRPWC(+57)FTTDPNKR | 9 | MG-HI; | 2.777863 | 5 | 2428.147 | 26.7036 |
| 223 | | | MG-DH | | | | |
| 220/ | NPDRELRPWC(+57)FTTDPNKR | 8 | MG-HI; | 3.910948 | 3 | 2410.132 | 25.60333 |
| 223 | | | MG-HI | | | | |
| 220/ | NPDRELRPWC(+57)FTTDPNKR | 3 | MG-DH; | 3.54006 | 4 | 2446.158 | 27.03834 |
| 223 | | | MG-DH | | | | |
| 234 | RWELC(+57)DIPR0 | 2 | MG-DH | 3.08503 | 3 | 1316.643 | 30.26445 |
| 242 | WELC(+57)DIPRC(+57) | 6 | MG-DH | 3.87702 | 3 | 2935.343 | 31.45411 |
| | TTPPPSSGPT | | | | | | |
| | YQC(+57)LK | | | | | | |
| 242 | WELC(+57)DIPRC(+57) | 3 | MG-HI | 3.994577 | 3 | 2917.329 | 30.77023 |
| | TTPPPSSGPT | | | | | | |
| | YQC(+57)LK | | | | | | |
| 265 | GTGENYRGNVAVTVSGHTC | 7 | MG-DH | 5.357526 | 4 | 3631.651 | 20.37024 |
| | (+57)QHWSAQTPHTHNR | | | | | | |
| 265 | GTGENYRGNVAVTVSGHTC | 5 | MG-HI | 6.999237 | 4 | 3613.643 | 21.54209 |
| | (+57)QHWSAQTPHTHNR | | | | | | |
| 290 | GNVAVTVSGHTC(+57) | 12 | MG-HI | 5.533076 | 4 | 3809.751 | 22.13035 |
| | QHWSAQTPH | | | | | | |
| | THNRTPENFPC(+57)K | | | | | | |
| 290 | GNVAVTVSGHTC(+57) | 4 | MG-HI | 3.690379 | 4 | 3827.75 | 21.13519 |
| | QHWSAQTPH | | | | | | |
| | THNRTPENFPC(+57)K | | | | | | |
| 312 | RAPWC(+57)HTTNSQVR | 10 | MG-DH | 3.530581 | 3 | 1684.793 | 20.30097 |
| 312 | RAPWC(+57)HTTNSQVR | 8 | MG-HI | 4.33057 | 2 | 1666.784 | 15.17595 |
| 324 | RAPWC(+57)HTTNSQVRWEYC | 7 | MG-HI | 5.757808 | 3 | 2433.099 | 23.69433 |
| | (+57)K | | | | | | |
| 324 | RAPWC(+57)HTTNSQVRWEYC | 4 | MG-DH | 4.016407 | 3 | 2451.104 | 22.92327 |
| | (+57)K | | | | | | |
| 324 | APWC(+57)HTTN SQVRWEYC | 3 | MG-HI | 4.331403 | 3 | 2276.992 | 23.76159 |
| | (+57)K | | | | | | |
| 312/ | RAPWC(+57)HTTNSQVRWEYC | 7 | MG-DH; | 4.502779 | 4 | 2505.118 | 25.38008 |
| 324 | (+57)K | | MG-HI | | | | |
| 312/ | RAPWC(+57)HTTNSQVRWEYC | 3 | MG-DH; | 3.028201 | 4 | 2523.122 | 24.30186 |
| 324 | (+57)K | | MG-DH | | | | |
| 312/ | RAPWC(+57)HTTNSQVRWEYC | 2 | MG-HI; | 4.103342 | 4 | 2487.099 | 22.28137 |
| 324 | (+57)K | | MG-HI | | | | |
| 389 | C(+57)QSWSSMTPHRHQK | 4 | MG-HI | 3.011286 | 2 | 1823.808 | 16.3436 |
| 389 | C(+57)QSWSSMTPHRHQK | 2 | MG-DH | 2.834358 | 3 | 1841.816 | 19.05036 |
| 389 | KC(+57)QSWSSMTPHRHQK | 1 | MG-HI | 2776672 | 3 | 1951.899 | 16.78563 |
| 389 | KC(+57)QSWSSMTPHRHQK | 1 | MG-DH | 2.129934 | 4 | 1969.91 | 17.13416 |
| 474 | GKRATTVTGTPC(+57) | 4 | MG-HI | 3.84241 | 4 | 2521.189 | 19.80755 |
| | QDWAAQEPHR | | | | | | |
| 474 | RATTVTGTPC(+57) | 3 | MG-HI | 5.947902 | 3 | 2336.079 | 21.06519 |
| | QDWAAQEPHR | | | | | | |
| 474 | RATTVTGTPC(+57) | 3 | MG-DH | 5.884814 | 3 | 2354.094 | 23.31571 |
| | QDWAAQEPHR | | | | | | |
| 474 | GKRATTVTGTPC(+57) | 1 | MG-DH | 4.187221 | 4 | 2539.198 | 20.18088 |
| | QDWAAQEPHR | | | | | | |
| 493 | ATTVTGTPC(+57) | 6 | MG-HI | 3.009536 | 3 | 3459.609 | 25.66775 |
| | QDWAAQEPHRHSI | | | | | | |
| | FTPETNPR | | | | | | |
| 493 | RATTVTGPC(+57) | 4 | MG-HI | 7.000995 | 4 | 3615.708 | 24.01152 |
| | QDWAAQEPHRHS | | | | | | |
| | IFTPETNPR | | | | | | |
| 493 | ATTVTGTPC(+57) | 4 | MG-DH | 3.724425 | 3 | 3477.627 | 27.2342 |
| | QDWAAQEPHRHSI | | | | | | |
| | FTPETNPR | | | | | | |
| 493 | RATTVTGPC(+57) | 3 | MG-DH | 5.619551 | 4 | 3633.726 | 25.99322 |
| | QDWAAQEPHRHS | | | | | | |
| | IFTPETNPR | | | | | | |
| 474/ | RATTVTGTPCQDWAAQEPHR | 1 | MG-DH; | 4.9038 | 4 | 3630.747 | 25.5692 |
| 493 | HSIFTPETNPR | | MG-HI | | | | |
| 474/ | RATTVTGTPC(+57) | 1 | MG-HI; | 4.301919 | 4 | 3669.726 | 24.54209 |
| 493 | QDWAAQEPHRH | | | | | | |
| | SIFTPETNPR | | MG-HI | | | | |
| 504 | HSIFTPETNPRAGLEK | 16 | MG-HI | 3.900824 | 3 | 1850.936 | 21.85166 |
| 504 | HSIFTPETNPRAGLEK | 9 | MG-DH | 3.767757 | 3 | 1868.945 | 22.65094 |
| 489/ | ATTVTGTPC(+57) | 1 | MG-HI; | 2.587048 | 5 | 4029.912 | 26.15854 |
| 504 | QDWAAQEPHRHSI | | MG-DH | | | | |
| | FTPETNPRAGLEK | | | | | | |
| 530 | NPDGDVGGPWC(+57)YTTNPRK | 12 | MG-HI | 4.458975 | 2 | 2087.923 | 25.93358 |
| 530 | NPDGDVGGPWC(+57)YTTNPRK | 9 | MG-DH | 4.080699 | 2 | 2105.931 | 25.01715 |
| 530 | NYC(+57)RNPDGDVGGPWC | 3 | MG-HI | 5.674226 | 3 | 2681.16 | 25.34793 |
| | (+57)YTTNPRK | | | | | | |
| 561 | KC(+57)PGRVVGGC(+57) | 12 | MG-HI | 6.14196 | 4 | 2824.401 | 26.70944 |
| | VAHPHSWPWQVSLR | | | | | | |
| 561 | KC(+57)PGRVVGGC(+57) | 10 | MG-DH | 4.014128 | 4 | 2842.415 | 29.47906 |
| | VAHPHSWPWQVSLR | | | | | | |
| 561 | C(+57)PGRVVGGC(+57) | 5 | MG-DH | 5.375551 | 4 | 2714.314 | 30.75051 |
| | VAHPHSWPWQVSLR | | | | | | |
| 561 | C(+57)PGRVVGGC(+57) | 5 | MG-HI | 5.081058 | 4 | 2696.31 | 28.89118 |
| | VAHPHSWPWQVSLR | | | | | | |
| 561 | KCPGRVVGGCVAHPHSWPWQVSLR | 1 | MG-DH | 2.348458 | 4 | 2728.364 | 26.34512 |
| 561 | CPGRVVGGCVAHPHSWPWQVSLR | 1 | MG-DH | 2.001367 | 4 | 2600.271 | 28.38697 |
| 582 | TRFGMHFC(+57) | 1 | MG-HI | 3.315366 | 4 | 3172.525 | 39.77992 |
| | GGTLISPEWVLTAA | | | | | | |
| | HC(+57)LEK | | | | | | |
| 582 | TRFGM(+16)HFC(+57) | 1 | MG-HI | 3.058828 | 4 | 3188.512 | 38.4995 |
| | GGTLISPEWV | | | | | | |
| | LTAAHC(+57)LEK | | | | | | |
| 582 | TRFGMHFC(+57) | 1 | MG-DH | 2.227959 | 4 | 3190.524 | 40.76536 |
| | GGTLISPEWVLTAA | | | | | | |
| | HC(+57)LEK | | | | | | |
| 644 | LFLEPTRK | 2 | MG-HI | 2.553553 | 2 | 1057.603 | 23.37511 |
| 712 | VC(+57)NRYEFLNGR | 5 | MG-DH | 3.733632 | 3 | 1499.702 | 24.17667 |
| 712 | VC(+57)NRYEFLNGR | 4 | MG-HI | 3.763921 | 2 | 1481.693 | 23.108 |
| 767 | DKYILQGVTSWGLGC(+57) | 11 | MG-HI | 5.617276 | 4 | 2988.549 | 31.59185 |
| | ARPNKPGVYVR | | | | | | |
| 767 | DKYILQGVTSWGLGC(+57) | 8 | MG-DH | 5.893369 | 4 | 3006.557 | 32.86298 |
| | ARPNKPGVYVR | | | | | | |
| 767 | YILQGVTSWGLGC(+57) | 6 | MG-DH | 5.086669 | 3 | 2763.443 | 33.69309 |
| | ARPNKPGVYVR | | | | | | |
| 767 | YILQGVTSWGLGC(+57) | 6 | MG-HI | 4.357009 | 3 | 2745.433 | 32.3703 |
| | ARPNKPGVYVR | | | | | | |
| 779 | VSRFVTWIEGVM(+16)R | 9 | MG-HI | 3.987265 | 3 | 1649.847 | 34.02246 |
| 779 | VSRFVTWIEGVM(+16)R | 5 | MG-DH | 2.883295 | 3 | 1667.857 | 35.16557 |
| 779 | VSRFVTWIEGVMR | 4 | MG-HI | 3.525512 | 2 | 1633.854 | 39.53223 |
| 779 | VSRFVTWIEGVMR | 2 | MG-DH | 2.12907 | 2 | 1651.863 | 41.29859 |
| 789 | FVTWIEGVMRNN | 14 | MG-HI | 4.085983 | 2 | 1519.736 | 37.34363 |
| 789 | FVTWIEGVMRNN | 13 | MG-DH | 3.926203 | 2 | 1537.748 | 38.46862 |
| 789 | FVTWIEGVM(+16)RNN | 10 | MG-HI | 4.154977 | 2 | 1535.731 | 32.24466 |
| 789 | FVTWIEGVM(+16)RNN | 6 | MG-DH | 3.589212 | 2 | 1553.74 | 31.4466 |
| 779/ | VSRFVTWIEGVM(+16)RNN | 3 | MG-HI; | 3.710465 | 3 | 1949.954 | 34.73159 |
| 789 | | | MG-DH | | | | |
| 779/ | VSRFVTWIEGVM(+16)RNN | 3 | MG-HI; | 3.59701 | 3 | 1931.944 | 34.17961 |
| 789 | | | MG-HI | | | | |
| 779/ | VSRFVTWIEGVM(+16)RNN | 1 | MG-DH; | 2.901959 | 3 | 1967.958 | 36.23294 |
| 789 | | | MG-DH | | | | |

Identified sites of MG adduction on plasminogen with 24 hr, 500 μM MG incubation, either as MG-DH or MG-H1. Peptides with methionine oxidation (+16) and cysteine carbamidomethylation (+57) are indicated as such. n=3.

TABLE 13
Pg-tPA interaction energy changes due to modification.

| | Interaction | Interaction energy | Interaction | Net change in |
|---|---|---|---|---|
| | energy (steric | (electrostatic | energy | interaction |
| Protein-Protein | component) | component) | TOTAL | energy |
| Interaction type | kcal/mol | kcal/mol | kcal/mol | kcal/mol |
| tPA-Pg | −200.1 | −625.2 | −825.4 | |
| tPA-Pg MG-H1 | −188.3 | −411.4 | −599.8 | 225.6 |
| adduct on R561 | | | | |
| tPA-Pg | −209.8 | −205.2 | −415 | 410.4 |
| argpyrimidine | | | | |
| adduct on R561 | | | | |
| TPA-plasminogen | −206.7 | −386.5 | −593.2 | 232.2 |
| 3DG-H1 adduct | | | | |
| on R561 | | | | |

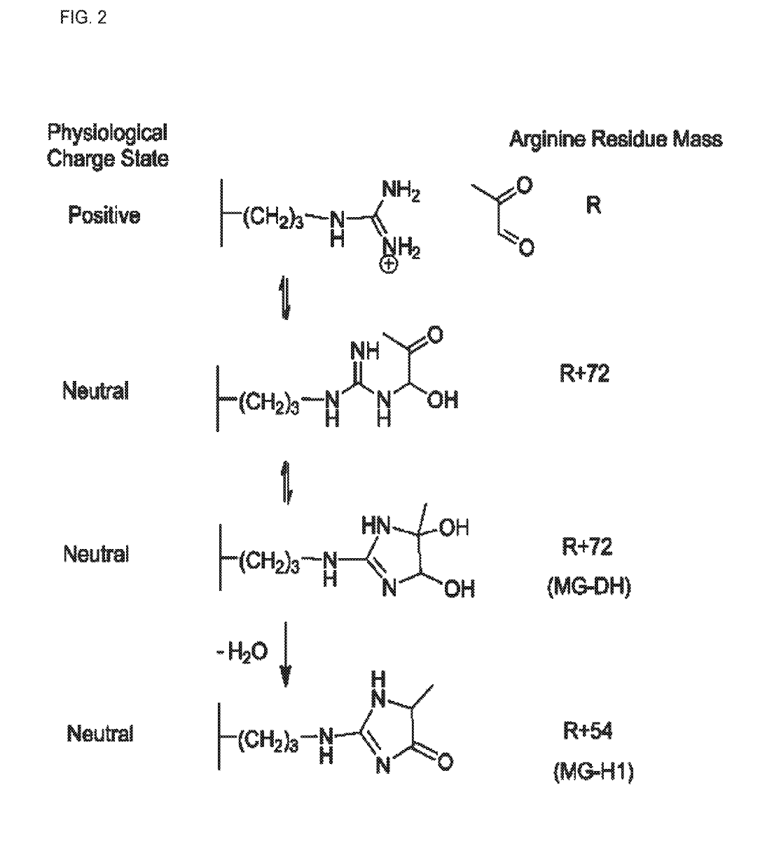

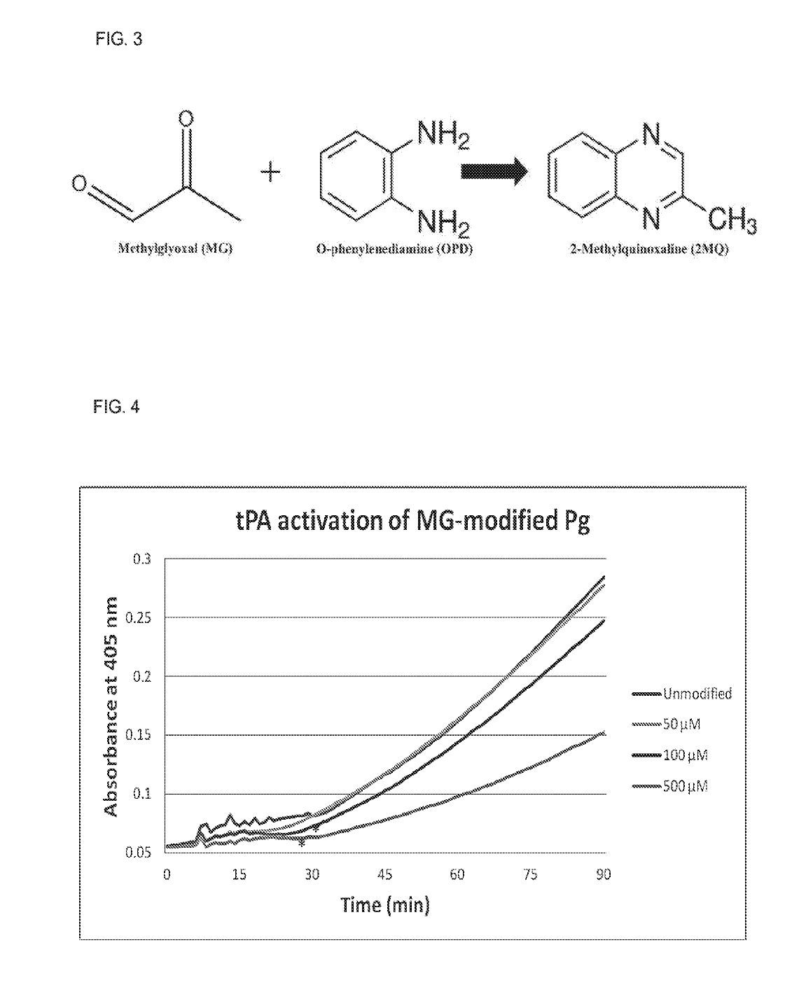

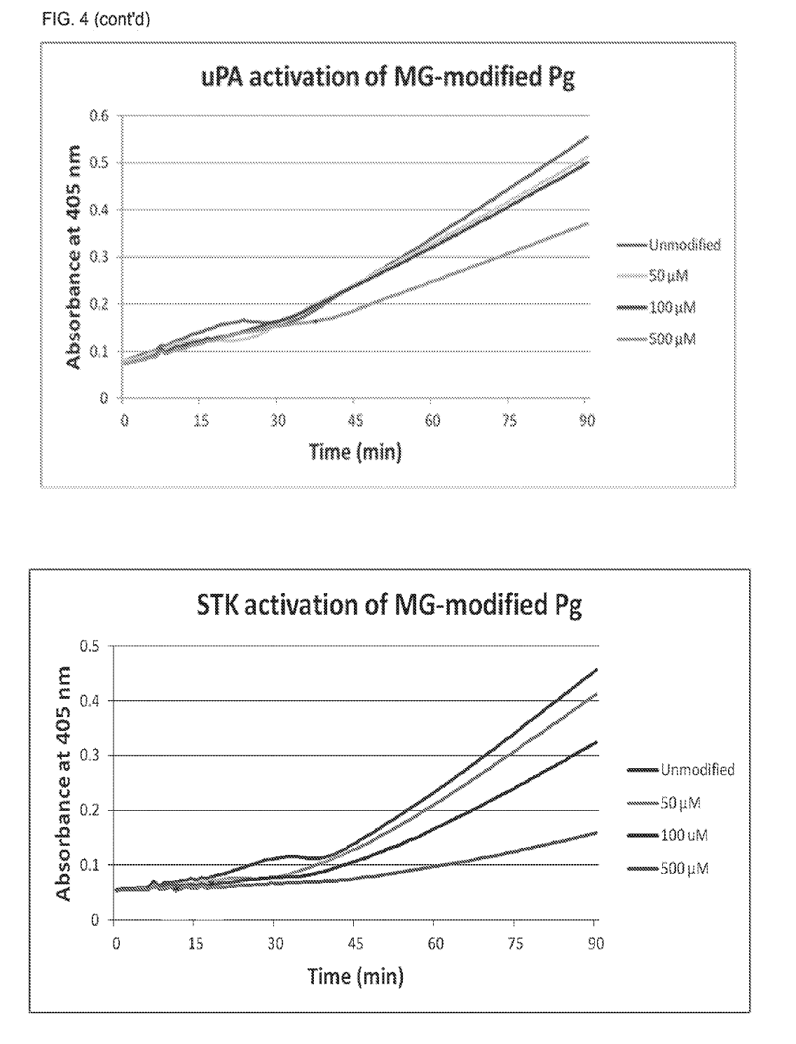

A molecular model of Pg-tPA was examined for energy changes due to MG-H1, argpyrimidine, or 3DG-H1 adduction (see FIG. 2 for structures). Both the steric and electrostatic components were analyzed for changes. Energy values were obtained using Affinity docking module.

### Example 5 References

Ahmed, N., Argirov, O. K., Minhas, H. S., Cordeiro, C. A., and Thornalley, P. J. (2002). Assay of advanced glycation endproducts (AGEs): surveying AGEs by chromatographic assay with derivatization by 6-aminoquinolyl-N-hydroxysuccinimidyl-carbamate and application to Nepsilon-carboxymethyl-lysine- and Nepsilon-(1-carboxyethyl)lysine-modified albumin. Biochem. J. 364, 1-14.
Ahmed, N., Thornalley, P. J., Dawczynski, J., Franke, S., Strobel, J., Stein, G., and Haik, G. M. (2003). Methylglyoxal-Derived Hydroimidazolone Advanced Glycation End-Products of Human Lens Proteins. Investigative Ophthalmology & Visual Science. 44, 5287-5292.
Ajjan, R. A., Gamlen, T., Standeven, K. F., Mughal, S., Hess, K., Smith, K. A., Dunn, E. J., Anwar, M. M., Rabbani, N., Thornalley, P. J., Philippou, H., and Grant, P. J. (2013). Diabetes is associated with posttranslational modifications in plasminogen resulting in reduced plasmin generation and enzyme-specific activity. Blood. 122, 134-142.
Andon, N. L., Hollingworth, S., Koller, A., Greenland, A. J., Yates, J. R., 3rd, and Haynes, P. A. (2002). Proteomic characterization of wheat amyloplasts using identification of proteins by tandem mass spectrometry. Proteomics. 2, 1156-1168.
Battistel, M. D., Grishaev, A., An, S. S., Castellino, F. J., and Llinas, M. (2009). Solution structure and functional characterization of human plasminogen kringle 5. Biochemistry. 48, 10208-10219.
Benfeldt, C., Larsen, L. B., Rasmussen, J. T., Andreasen, P. A., and Petersen, T. E. (1995). Isolation and characterization of plasminogen and plasmin from bovine milk. Int. Dairy J. 5, 577-592.
Blum, H., Beier, H., and Gross, H. J. (1987). Improved silver staining of plant proteins, RNA and DNA in polyacrylamide gels. Electrophoresis. 8, 93-99.
Chaplen, F. W., Fahl, W. E., and Cameron, D. C. (1998). Evidence of high levels of methylglyoxal in cultured Chinese hamster ovary cells. Proc. Natl. Acad. Sci. U.S.A 95, 5533-5538.
Chowdhury, M. A. R., Hossain, A. M., Dey, S. R., and Akhtaruzzaman, A. (2008). A comparative study on the effect of streptokinase between diabetic and non-diabetic myocardial infarction patients. Bangladesh Journal of Pharmacology. 3, 1-7.
Gorina, Y. and Lentzner, H. (2008). Multiple causes of death in old age. Aging Trends. (9), 1-9.
Gugliucci, A. (2003). A practical method to study functional impairment of proteins by glycation and effects of inhibitors using current coagulation/fibrinolysis reagent kits. Clin. Biochem. 36, 155-158.
Heron, M. (2007). Deaths: leading causes for 2004. Natl. Vital Stat. Rep. 56, 1-95.
Kall, L., Storey, J. D., MacCoss, M. J., and Noble, W. S. (2008). Posterior error probabilities and false discovery rates: two sides of the same coin. J. Proteome Res. 7, 40-44.
Kimzey, M. J., Yassine, H. N., Riepel, B. M., Tsaprailis, G., Monks, T. J., and Lau, S. S. (2011). New site(s) of methylglyoxal-modified human serum albumin, identified by multiple reaction monitoring, alter warfarin binding and prostaglandin metabolism. Chem. Biol. Interact. 192, 122-128.
Lerant, I., Kolev, K., Gombas, J., and Machovich, R. (2000). Modulation of plasminogen activation and plasmin activity by methylglyoxal modification of the zymogen. Biochim. Biophys. Acta. 1480, 311-320.
Loy, J. A., Lin, X., Schenone, M., Castellino, F. J., Zhang, X. C., and Tang, J. (2001). Domain Interactions between Streptokinase and Human Plasminogenâ€. Biochemistry (N.Y.). 40, 14686-14695.
Miyashita, C., Wenzel, E., and Heiden, M. (1988). Plasminogen: a brief introduction into its biochemistry and function. Haemostasis. 18 Suppl 1, 7-13.
Mustaffa, N., Ibrahim, S., Abdullah, W. Z., and Yusof, Z. (2011). Add-on rosiglitazone therapy improves plasminogen activity and high-density lipoprotein cholesterol in type 2 diabetes mellitus. Blood Coagul. Fibrinolysis. 22, 512-520.
Ofran, Y. and Rost, B. (2007). Protein-protein interaction hotspots carved into sequences. PLoS Comput. Biol. 3, e119.
Olmea, O. and Valencia, A. (1997). Improving contact predictions by the combination of correlated mutations and other sources of sequence information. Fold. Des. 2, S25-32.
Peisach, E., Wang, J., de los Santos, T., Reich, E., and Ringe, D. (1999). Crystal structure of the proenzyme domain of plasminogen. Biochemistry. 38, 11180-11188.
Plutzky, J. (2011). Macrovascular effects and safety issues of therapies for type 2 diabetes. Am. J. Cardiol. 108, 25B-32B.
Qian, W. J., Liu, T., Monroe, M. E., Strittmatter, E. F., Jacobs, J. M., Kangas, L. J., Petritis, K., Camp, D. G., 2nd, and Smith, R. D. (2005). Probability-based evaluation of peptide and protein identifications from tandem mass spectrometry and SEQUEST analysis: the human proteome. J. Proteome Res. 4, 53-62.
Renatus, M., Engh, R. A., Stubbs, M. T., Huber, R., Fischer, S., Kohnert, U., and Bode, W. (1997). Lysine 156 promotes the anomalous proenzyme activity of tPA: X-ray crystal structure of single-chain human tPA. EMBO J. 16, 4797-4805.
Shevchenko, A., Wilm, M., Vorm, O., and Mann, M. (1996). Mass spectrometric sequencing of proteins silver-stained polyacrylamide gels. Anal. Chem. 68, 850-858.
Spivak, M., Weston, J., Bottou, L., Kall, L., and Noble, W. S. (2009). Improvements to the percolator algorithm for Peptide identification from shotgun proteomics data sets. J. Proteome Res. 8, 3737-3745.
Wang, X., Terzyan, S., Tang, J., Loy, J. A., Lin, X., and Zhang, X. C. (2000). Human plasminogen catalytic domain undergoes an unusual conformational change upon activation. J. Mol. Biol. 295, 903-914.
Young, K. C., Shi, G. Y., Wu, D. H., Chang, L. C., Chang, B. I., Ou, C. P., and Wu, H. L. (1998). Plasminogen activation by streptokinase via a unique mechanism. J. Biol. Chem. 273, 3110-3116.
Zhang, Y., Liang, Z., Hsueh, H. T., Ploplis, V. A., and Castellino, F. J. (2012). Characterization of streptokinases from group A Streptococci reveals a strong functional relationship that supports the coinheritance of plasminogen-binding M protein and cluster 2b streptokinase. J. Biol. Chem. 287, 42093-42103.
Zhang, Y., Wisner, A., Maroun, R. C., Choumet, V., Xiong, Y., and Bon, C. (1997). Trimeresurus stejnegeri snake venom plasminogen activator. Site-directed mutagenesis and molecular modeling. J. Biol. Chem. 272, 20531-20537.

### Example 6

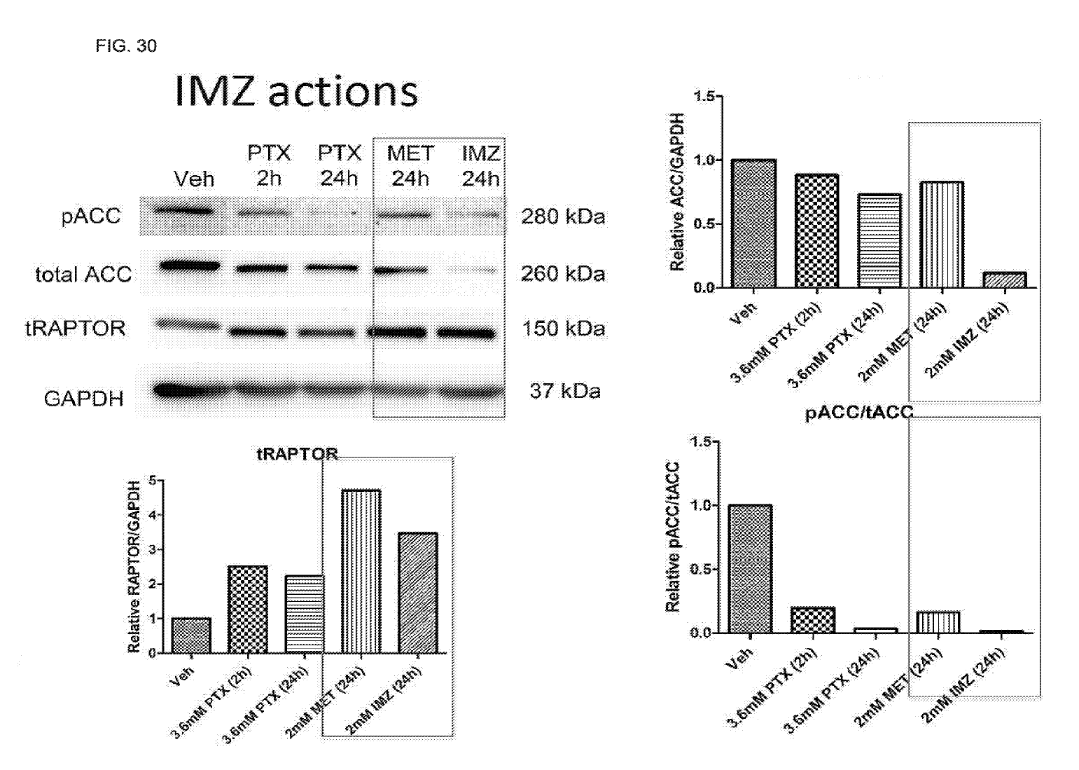

FIG. 30 shows the effect of imidazoline on tRAPTOR and AAC Acetyl-CoA carboxylase (ACC)-266 kD.

RAPTOR Involved in the control of the mammalian target of rapamycin complex 1 (mTORC1) activity which regulates cell growth and survival, and autophagy in response to nutrient and hormonal signals; functions as a scaffold for recruiting mTORC1 substrates-150 kD (FIG. 30).

### Example 7

Reactive dicarbonyls, such as methylglyoxal (MG), are elevated in type-2 diabetes mellitus (T2DM) patients, covalently modify proteins, and contribute to a number of diabetic complications. The T2DM first-line therapy metformin (MF) significantly reduces diabetic endpoints and mortality more effectively than other hypoglycemic agents. Experiments examined whether MF directly scavenges MG as an alternative mechanism of drug efficacy, in addition to its antigluconeogenesis mechanism. Experiments synthesized and characterized a MF/MG imidazolinone product (IMZ) by 13C, 1H and HMBC NMR (Bruker DRX-600), X-ray diffraction analysis (Bruker APEX-II CCD) as well as ESI-LC-MS/MS (MH+, 184 m/z; Agilent 6490). Using an LC/MS multiple reaction monitoring (MRM) method MF and the IMZ product in human urine were measured with sensitivity in the nM range. The IMZ was detected in all MF-treated T2DM patients and not detected in patients that have not taken metformin, as expected. Quantitation of IMZ in a cohort of >90 MF-treated patients is ongoing, utilizing specific gravity normalization. The data reveals that urine from every T2DM patient treated with MF contains the IMZ product as a result of a direct reaction with MG, and increased levels of MF directly correlate with elevations in IMZ. Determining if the toxicant MG is being reduced in a concomitant manner is also ongoing. The present work has identified an optimized method for detecting o-phenylenediamine derivatized MG (2MQ) using an LC/MS multiple reaction monitoring method. Utilizing this method, the 2MQ product can be detected at an average retention time of 4.3 minutes in a range between 0.01 μM to 5.5 μM without reaching saturation. MF may play a role in scavenging the highly reactive MG in vivo, in addition to lowering hepatic gluconeogenesis. The role of the IMZ in the reduction of diabetic complications warrants further study.

Reactive dicarbonyls such as methylglyoxal accumulate in diabetic patients due to elevated glucose as well as increased oxidative stress (FIG. 31). These toxic dicarbonyls directly damage proteins through adduction at arginine residues on proteins (advanced glycation end products [AGEs]) and are implicated in the progression of a number of type-2 diabetic complications, including cardiovascular disease (CVD). There is currently no therapy for directly reducing concentrations of these compounds in humans.

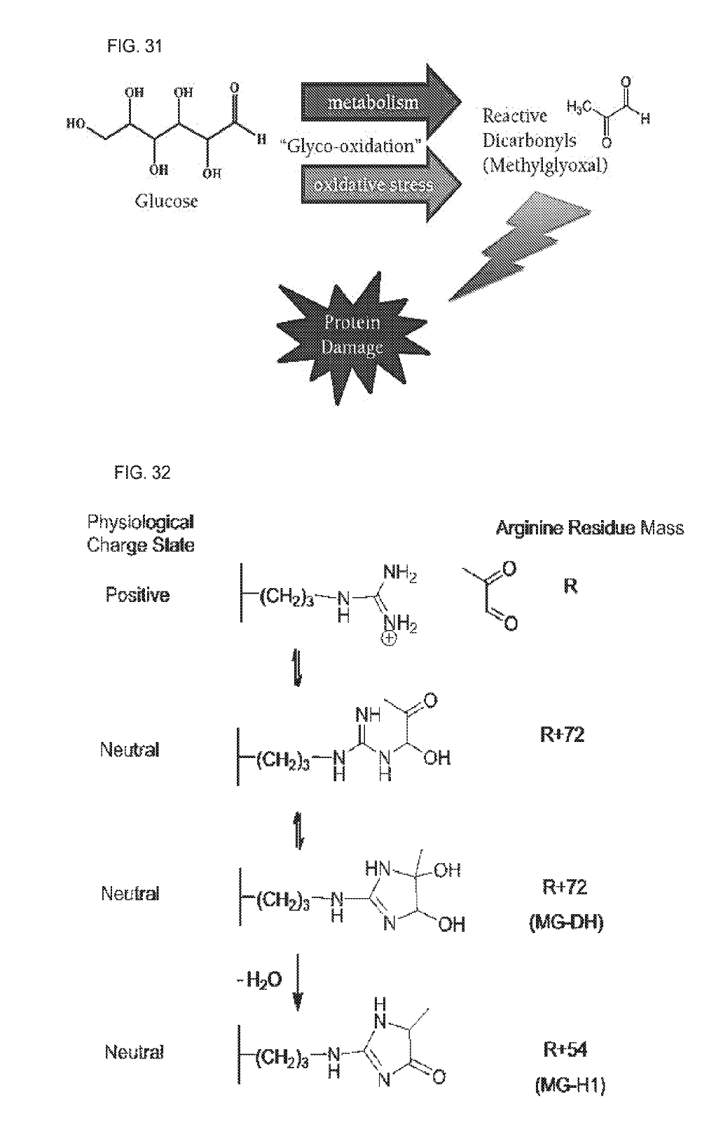

Among the primary causes of CVD in T2DM is the nonenzymatic formation of advanced glycation endproducts (AGEs) from reactive dicarbonyl sugars such as methylglyoxal (MG) and 3-deoxyglucosone (3DG). MG irreversibly adducts arginine residues in proteins which subsequently alter the charge status (FIG. 32) creating oxidative stress and an environment in which atherosclerosis is promoted.

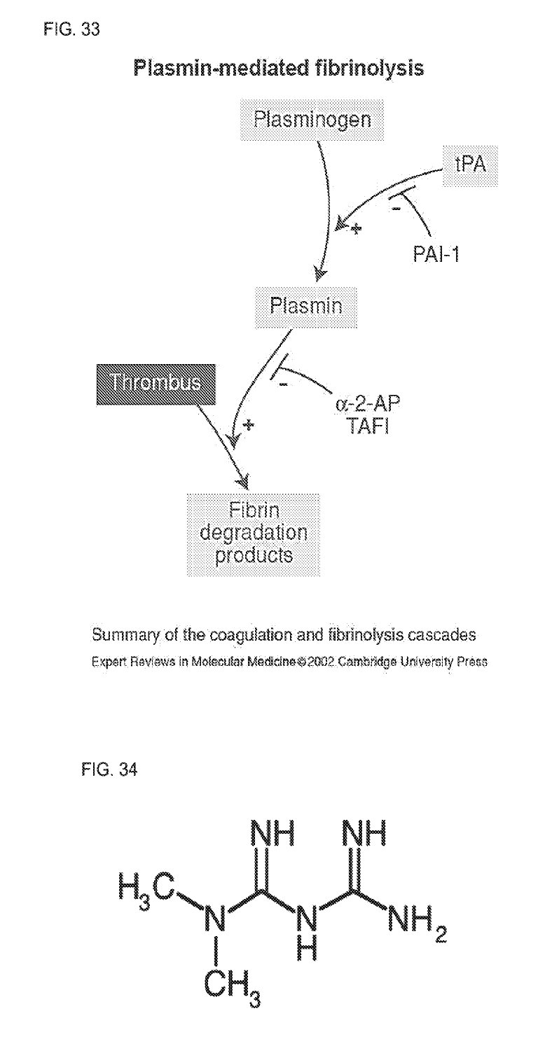

The process of thrombosis is normally counteracted by the process of fibrinolysis to maintain hemostasis (FIG. 33). Plasminogen (Pg), a zymogen released from the liver, is converted into plasmin by the enzyme tissue plasminogen activator (tPA) via cleavage between arginine-561 (R561) and valine-562 (V562). Plasmin is the active enzyme which degrades the fibrin backbone of a clot. In vivo fibrinolysis may be impaired as a result of MG modification of Pg. Due to the steric and electrostatic changes that MG adduction causes, it is contemplated that that functional impairment of normal hemostasis may be due to adduction of critical arginine(s) on Pg.

Direct scavenging by metformin of dicarbonyls has not been thoroughly studied despite its prevalence as a theory for decreased AGEs. Previous work theorized from limited data on a synthesized metformin-MG product that the cyclic product formed is a seven-membered triazepinone ring structure, while further work indicated that despite its possible scavenging mechanism, the kinetics of the reaction would perhaps result in a negligible effect in humans. While the kinetics previously put forth may indicate a slow reaction, the high amount of metformin taken by patients on a daily basis as well as the ever-present levels of MG in diabetics doesn't discount scavenging as a possible beneficial mechanism for diabetic patients taking metformin.

As previously mentioned, there is currently no therapy for directly reducing concentrations of these compounds in humans, and thereby preventing AGE-induced diabetic complications. Metformin (FIG. 34) is a first-line diabetic therapy that is used primarily because of its potent anti-hyperglycemic effects with little adverse side effects from the drug. The drug is unique among anti-hyperglycemic agents in that it is capable of reducing diabetic complications and overall mortality compared to other diabetic therapies. The drug has been linked to decreased AGEs in humans but the mechanism behind this link is yet to be elucidated. Reduced overall glucose burden, activation of glyoxalase enzyme system, and direct scavenging of dicarbonyls have all been proposed as mechanisms for this effect.

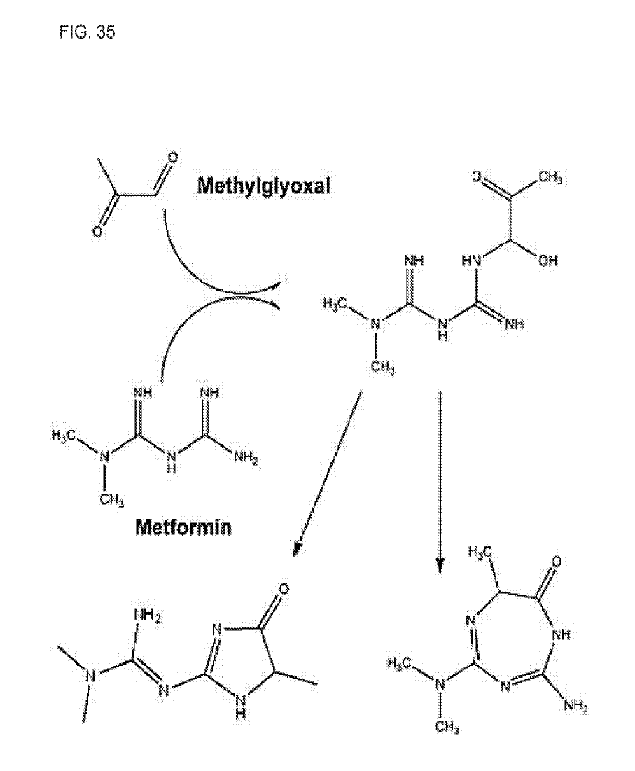

Experiments described herein identified metformin as a scavenger. Indeed, experiments successfully synthesized the MG-metformin IMZ with the published method from the Wiernsperger group, with a slight modification in order to neutralize the HCl salt in the compound at the start of the reaction. The group that first synthesized this product theorized that it was a seven-membered triazepinone ring structure. Experiments hypothesized that the more favorable product is the 5-membered imidazolinone structure (FIG. 35).

200 mM metformin HCL and 200 mM NaOH were mixed with 200 mM MG at 5° C. After being stirred for 1 hr at 5° C., the solution was stirred at 20° C. for the final four hours of the synthesis. The resulting product was filtered through a Whatman #1 filter and dried down.

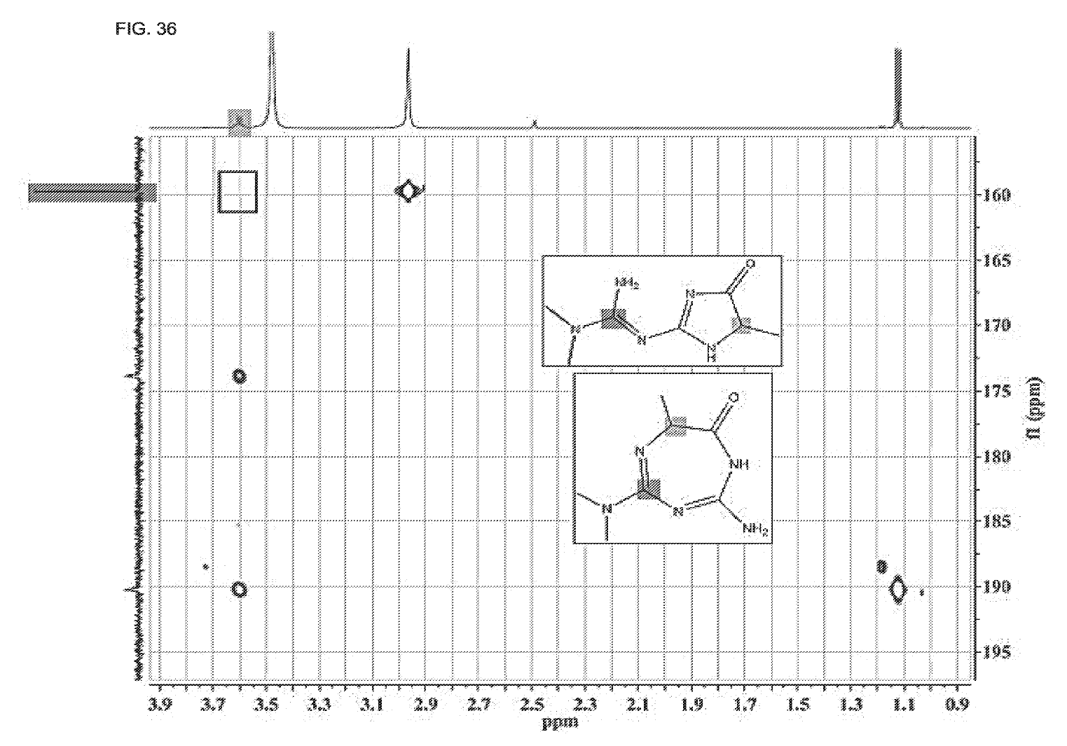

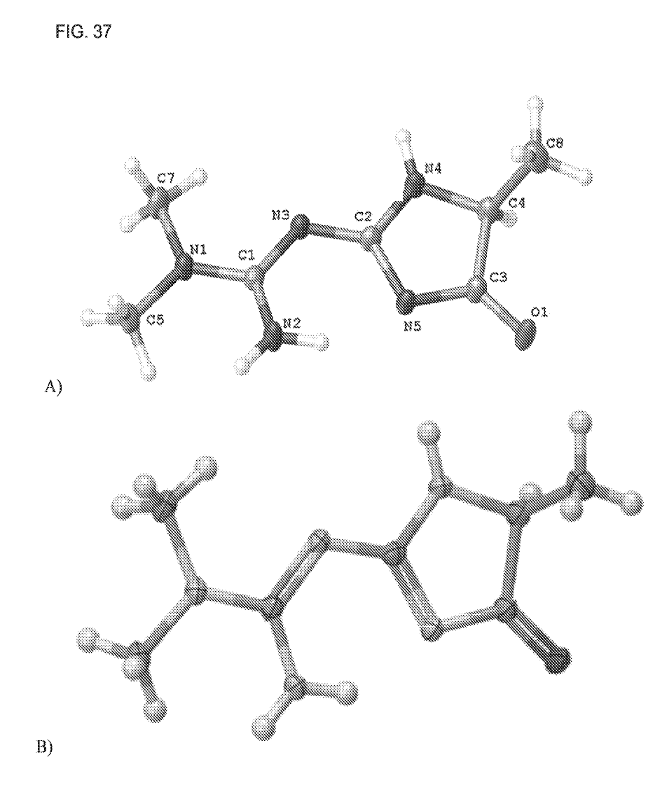

Heteronuclear multiple bond correlation (HMBC) NMR was the first data to indicate that the product is a five-membered IMZ and may not be the seven-membered triazepinone (FIG. 36). The x-axis indicates proton signals, while the y-axis indicates carbon signals. Correlation between a proton and a carbon signal up to three bonds away is indicated by a red signal in the NMR spectrum. The data indicate that the carbon marked in blue on both structures at 160 ppm, does not correlate with the quartet at 3.6 ppm (indicated by the absence of a peak within the dark blue box). This quartet is from the hydrogen at the carbon highlighted in green on both structures. In the seven-membered triazepinone ring structure, a correlation between these two peaks was expected, as they are within three bonds from each other (FIG. 37).

Molecule with displacement ellipsoids at the 50% probability level as determined by x-ray diffraction. B. Molecular skeleton of the 5-membered imidazolinone compound with assigned double bonds analyzed by Mercury. C1-N3 and C2-N5 were assigned as double bonds. This forms a conjugated system when the carbonyl bond is included. A search of similar structures in the Cambridge Data Base using Mercury reveals that the C2-N5 bond distance is about 0.04 Å longer than expected for this type of double bond. C1-N3 is also long and C2-N3 is short, but these distances are not statistically outside the range of expected bond distances. Taken together, they may imply some delocalization in the molecule.

IMZ levels correlate significantly with metformin concentrations.

24-hr sample collection indicates better IMZ linearity with metformin.

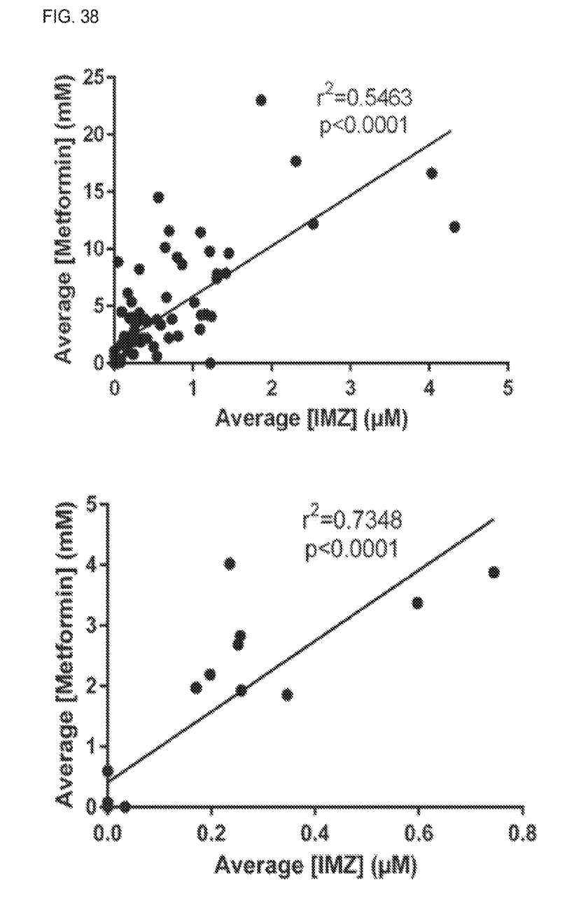

MRM analysis of 92 metformin-treated T2DM human urine samples demonstrates that average IMZ concentrations have a statistically significant positive linear correlation (r2=0.5463; p<0.0001) with metformin concentrations. Samples were run in triplicate and the average value was utilized (FIG. 38).

MRM analysis of eighteen 24-hr collections from metformin-treated T2DM human urine samples demonstrates that average IMZ concentrations have a statistically significant positive linear correlation (r2=0.7348; p<0.0001) with metformin concentrations. This linear correlation was closer to a 1:1 linearity than the overall cohort, and indicates that 24-hr pooled samples may yield better IMZ results. Samples were run in triplicate and the average value was utilized.

Metformin (MF) reacts with methylglyoxal (MG) to form a novel five-membered imidazolinone ring structure, with the structure unequivocally identified via HMBC NMR and x-ray diffraction.

Levels of the MF-MG adduct are detectable up to 4.32 μM/specific gravity in urine samples from human diabetic subjects on MF treatment. As expected, this product is only observable in patients actively taking the drug in compliance.

Analysis of 92 MF-treated human samples indicated a strong positive correlation between IMZ and metformin levels in patient urine.

Experiments conducted during the course of developing embodiments for the present invention resulted in detection of methyglyoxal in human urine.

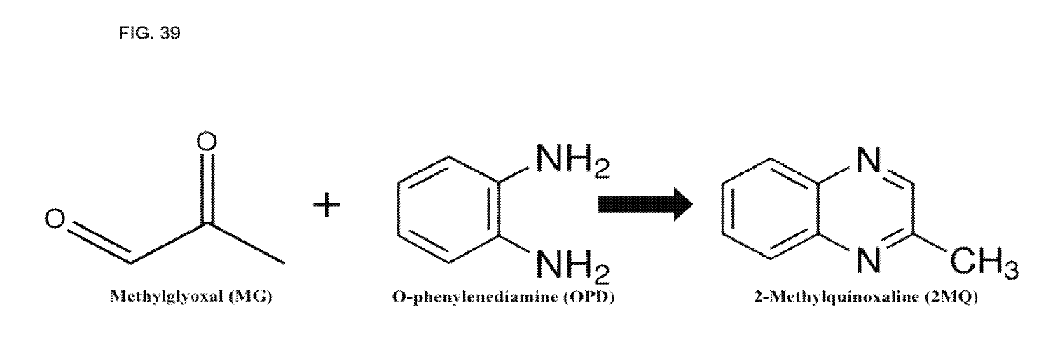

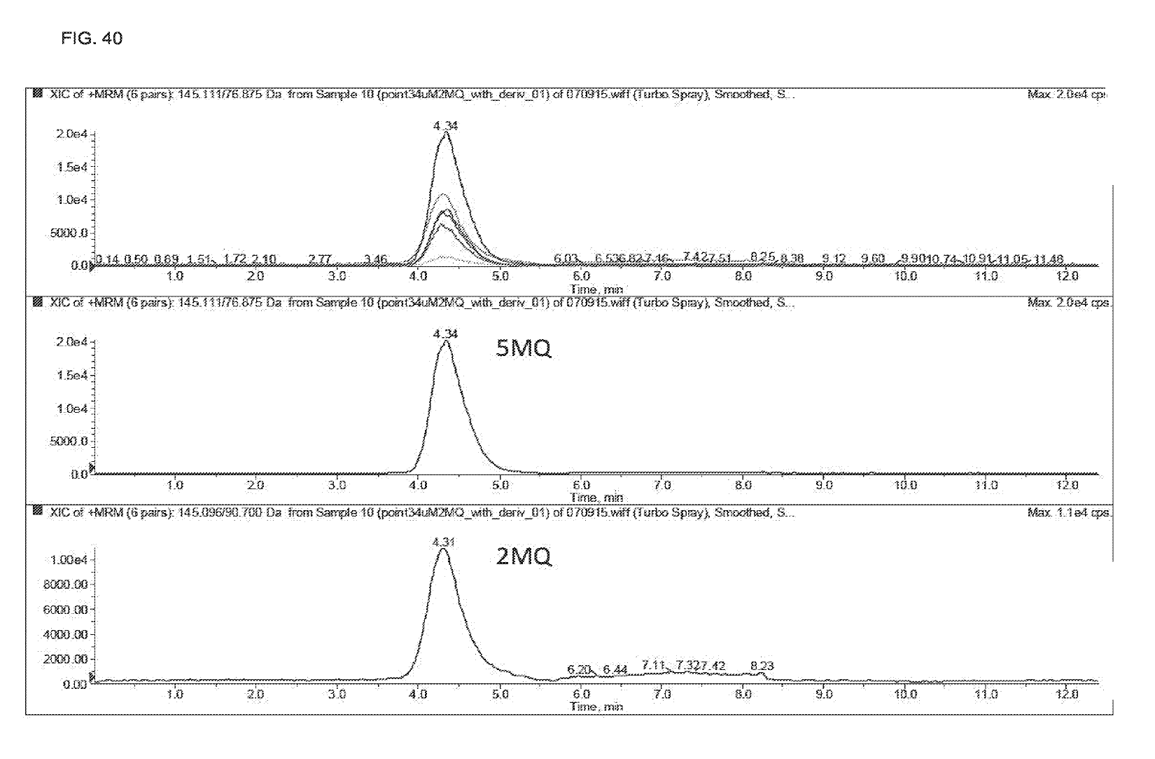

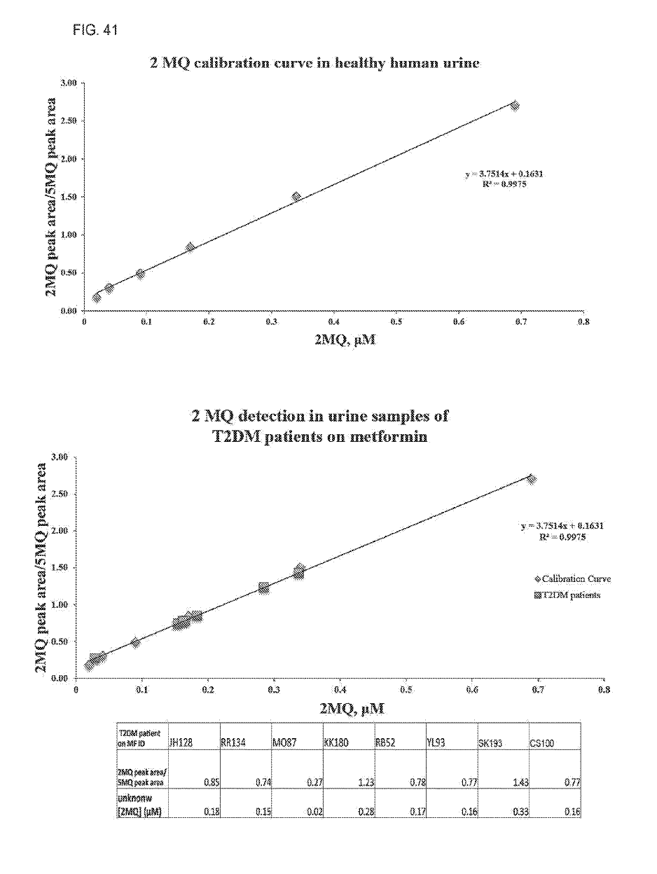

A multiple reaction monitoring (MRM) assay for the identification and quantitation of derivatized methylglyoxal (2MQ) levels in patient samples was developed using an AB SCIEX API 3000 triple-quadrupole mass spectrometer. Samples were spiked with the internal standard 5MQ and derivatized with o-phenylenediamine (reaction shown in FIG. 39) in a dark room for 4 hours to form the detectable 2MQ product (FIG. 40).

Samples were separated with a HILIC column. Parent ion to daughter fragment transitions were developed to be sensitive to the derivatized form of methylglyoxal (2MQ) and the internal standard 5MQ, which have a parent m/z of 145, but different daughter fragment transitions. All parent-daughter ion transitions co-elute at 4.31 minutes when 2MQ is present in the sample and all parent-daughter ion transitions co-elute at 4.34 minutes for 5MQ (shown in the representative human sample to the left). The following transitions are used for 2MQ: m/z 145->77 (quantitation) and 145->92 (confirmation). The following transitions are used for 5 MQ: m/z 145->91 (quantitation) and 145->117 (confirmation).

Concentrations of methylglyoxal from 0.01 μM to 5.5 μM and a known concentration of 5MQ were spiked into healthy patient urine that were not on metformin treatment. Great linearity was achieved with all 10 points of calibration; however only six points ranging from 0.01 μM to 0.7 μM were needed for the assay (FIG. 41).

Identification and quantitation of derivatized MG (2MQ) in human urine and plasma samples from the cohort of >90 diabetic patients treated with metformin is ongoing. For confirmation that the developed MRM method is suitable for patient samples, a selection of patients with comparable unrinary levels of metformin were used for analysis. The concentration of 2MQ found in these patients ranged from 0.02 μM to 0.33 μM, demonstrating that the method is sufficient to use for the remainder of analysis.

We can successfully detect the quinoxaline derivative of methylglyoxal (2MQ) at a range of 0.01 μM to 5.5 μM without reaching saturation by the MRM method.

Levels of 2MQ are detectable in urine samples from human diabetic subjects on metformin treatment within the range of calibration.

Analysis of a select few metformin-treated human samples within the same range of urinary metformin concentration indicated a similar range of 2MQ, but further analysis needs to be done to make that connection.

Determining if the toxicant MG is being reduced in a concomitant manner as IMZ is being reduced is still ongoing.

Patients taken off of MF due to lack of glycemic response may still benefit from MF due to this alternate mechanism, which could play a role in reducing diabetic complications.

Having now fully described the invention, it will be understood by those of skill in the art that the same can be performed within a wide and equivalent range of conditions, formulations, and other parameters without affecting the scope of the invention or any embodiment thereof. All patents, patent applications and publications cited herein are fully incorporated by reference herein in their entirety.

### INCORPORATION BY REFERENCE

The entire disclosure of each of the patent documents and scientific articles referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

1. A method for determining the efficacy of metformin (MF) treatment in a patient, comprising
a) obtaining a urine sample from the patient;
b) detecting whether a MF/methylglyoxal (MG) product is present in the urine sample by contacting the urine sample with an agent that binds a MF/MG product and detecting binding between the MF/MG product and agent that binds a MF/MG product; and
c) determining the MF treatment as efficacious when the presence of the MF/MG product in the urine sample is detected.

2. The method of claim 1, wherein the patient is a human patient.

3. The method of claim 1, wherein the patient is a human patient diagnosed with type 2 diabetes (T2D).

4. The method of claim 1, wherein the MF/MG product is a MF/MG imidazolinone product (IMZ).

5. The method of claim 1, wherein the agent that binds a MF/MG product is an anti-MF/MG product antibody.

6. The method of claim 1, wherein the agent that binds a MF/MG product is an anti-MF/MG product small molecule.

7. A method of diagnosing impaired fibrinolysis in a patient, comprising
a) obtaining a biological sample from the patient,
b) detecting whether an methylglyoxal (MG) modified plasminogen (Pg) product is present in the biological sample by contacting the biological sample with an agent that binds a MG/Pg product and detecting binding between the MG/Pg product and agent that binds a MG/Pg product; and
c) diagnosing impaired fibrinolysis in the patient when the presence of the MG/Pg product in the biological sample is detected.

8. (canceled)

9. The method of claim 7, wherein the patient is a human patient diagnosed with type 2 diabetes (T2D).

10. The method of claim 7, wherein the agent that binds a MG/Pg product is an anti-MG/Pg product antibody.

11. The method of claim 7, wherein the agent that binds a MG/Pg product is an anti-MG/Pg product small molecule.

12. The method of claim 7, wherein detected fibrinolysis is indicative of thrombosis.

13-23. (canceled)

24. A method of assaying a sample from a subject for the presence of advanced glycation end products (AGE), comprising:
a) contacting said sample with an assay for determining dicarbonyl modification and/or oxidation of serum albumin; and
b) determining the presence of dicarbonyl modification of one or more arginine residues on said serum albumin and/or oxidation of one or more methionine residues on said serum albumin.

25. The method of claim 24, wherein said arginine residues are one or more of R186, R257, and R428.

26. The method of claim 24, wherein said assay is a mass spectrometry assay.

27. The method of claim 24, wherein said subject has type 2 diabetes.

28. The method of claim 27, wherein said subject is currently taking metformin.

29. The method of claim 27, wherein said dicarbonyl modification is increased in said subjects with type 2 diabetes relative to subjects not having type 2 diabetes.

30. The method of claim 28, wherein said dicarbonyl modification is decreased in said subjects taking metformin relative to the level in subjects with type 2 diabetes not taking metformin.

31. The method of claim 30, wherein said decrease in dicarbonyl modification is indicative of said metformin being an effective treatment for said type 2 diabetes.

32. The method of any one of claim 24, wherein said dicarbonyl is selected from the group consisting of methylglyoxal, 3-deoxyglucosone, and glucosone.

33-69. (canceled)
